(19) United States
(12) Patent Application Publication
(10) Pub. No.: US 2007/0004684 A1
(43) Pub. Date: Jan. 4, 2007

(54) Alpha-Carbolines as CDK-1 inhibitors

(75) Inventors: Peter Sennhenn, Muenchen (DE); Andreas Mantoulidis, Vienna (AT); Matthias Treu, Vienna (AT); Ulrike Tontsch-Grunt, Baden (AT); Walter Spevak, Oberrohrbach (AT); Darryl McConnell, Vienna (AT); Andreas Schoop, Vienna (AT); Ralph Brueckner, Vienna (AT); Albrecht Jacobi, Frankfurt (DE); Ulrich Guertler, Vienna (AT); Gisela Schnapp, Biberach-Rindenmoos (DE); Christian Klein, Vienna (AT); Frank Himmelsbach, Mittelbiberach (DE); Alexander Pautsch, Ulm (DE); Bodo Betzmeier, Vienna (AT); Lars Herfurth, Vienna (AT); Juergen Mack, Biberach-Mettenberg (DE); Dieter Wiedenmayer, Biberach (DE); Gerd Bader, Vienna (AT); Ulrich Reiser, Vienna (AT)

Correspondence Address: MICHAEL P. MORRIS;BOEHRINGER INGELHEIM CORPORATION, 900 RIDGEBURY RD, P. O. BOX 368, RIDGEFIELD, CT, 06877-0368, US

(73) Assignee: Boehringer Ingelheim International GmbH, Ingelheim (DE)

(21) Appl. No.: 11/423,008

(22) Filed: Jun. 8, 2006

(30) Foreign Application Priority Data
Jun. 9, 2005 (EP) EP 05105051
Jun. 9, 2005 (EP) EP 05105052
Jun. 9, 2005 (EP) EP 05105054

(51) Int. Cl. A61K 31/4745; C07D 471/02; A61K 31/655

(52) U.S. Cl. 514/150; 514/151; 514/291; 546/86

(57) ABSTRACT

The present invention encompasses compounds of general formula (1) 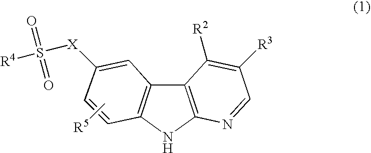 wherein
R² to R⁵ and X are defined as in claim 1, which are suitable for the treatment of diseases characterised by excessive or abnormal cell proliferation, and the use thereof for preparing a pharmaceutical composition having the above-mentioned properties.

The present invention relates to new α-carbolines of general formula (1) 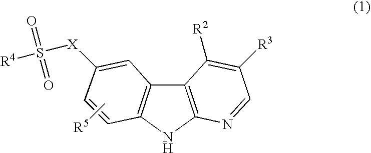
wherein the groups R² to R⁵ and X have the meanings given in the claims and specification, the isomers thereof, processes for preparing these α-carbolines and their use as pharmaceutical compositions.

### BACKGROUND TO THE INVENTION

Cyclin-dependent kinase (CDK) inhibitors play a crucial role in regulating the passage of eukaryotic cells through the cell cycle. By associating with regulatory sub-units, the cyclins, and by corresponding phosphorylation, cyclin-dependent kinases are activated. Interaction with CDK inhibitors inhibits the activity of the CDKs and leads to cell cycle arrest at the corresponding “checkpoint” in the cell cycle and to programmed cell death. A particularly suitable target molecule for developing substances for use in cancer therapy is the CDK1 receptor. This protein controls the final checkpoint in the cell cycle between the G2 and M phase. Intervention with the CDK1/cyclin B complex by means of inhibitory substances leads to the arresting of the proliferating cells in the G2 phase and finally to cell death.

The aim of the present invention is to point out new active substances which may be used for the prevention and/or treatment of diseases characterised by excessive or abnormal cell proliferation.

### DETAILED DESCRIPTION OF THE INVENTION

It has been found that, surprisingly, compounds of general formula (1) wherein the groups R² to R⁵ and X are defined as hereinafter act as inhibitors of specific cell cycle kinases. Thus, the compounds according to the invention may be used for example for the treatment of diseases associated with the activity of specific cell cycle kinases and characterised by excessive or abnormal cell proliferation.

The present invention relates to compounds of general formula (1) 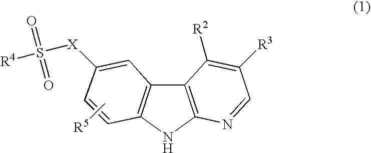
wherein
X equals O, NR¹ or CHR¹, and
R¹ denotes a group selected from among hydrogen, C₁₋₃alkyl and C₁₋₃haloalkyl, and
R² and R³ each independently of one another denote hydrogen or a group selected from among R^{a}, R^{b} and R^{a} substituted by one or more identical or different R^{b} and/or R^{c} and
R⁴ denotes —NR^{c}R^{c} or a group, optionally substituted by one or more R⁶, selected from among C₁₋₆alkyl, C₃₋₁₀cycloalkyl, 3-8 membered heterocyclyl, C₆₋₁₄aryl and 5-15 membered heteroaryl, and
R⁵ denotes a group selected from among hydrogen, halogen, C₁₋₃alkyl and C₁₋₃haloalkyl, and
R⁶ denotes a group selected from among R^{a}, R^{b} and R^{a} substituted by one or more identical or different R^{b} and/or R^{c}, and
each R^{a} denotes independently of one another selected from among C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocyclyl, 4-14 membered heterocyclylalkyl, 5-10 membered heteroaryl and 6-16 membered heteroarylalkyl, and
each R^{b} denotes a suitable group and each independently of one another denote selected from among ═O, —OR^{d}, C₁₋₃haloalkyloxy, —OCF₃, ═S, —SR^{d}, ═NR^{d}, ═NOR^{d}, —NR^{c}R^{c}, halogen, —CF3, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(S)R^{d}, —C(O)OR^{d}, —C(O)NR^{c}R^{c}, —C(O)NR^{d}OR^{d}, —C(O)N(R^{d})NR^{c}R^{c}, —CN(R^{d})NR^{c}R^{c}, —CN(OH)R^{d}, —CN(OH)NR^{c}R^{c}, —OC(O)R^{d}, —OC(O)OR^{d}, —OC(O)NR^{c}R^{c}, —OCN(R^{d})NR^{c}R^{c}, —N(R^{d})C(O)R^{d}, —N(R^{d})C(S)R^{d}, —N(R^{d})S(O)₂R^{d}, —N(R^{d})C(O)OR^{d}, —N(R^{d})C(O)NR^{c}R^{c}, and —N(R^{d})C(NR^{d})NR^{c}R^{c}, and
each R^{c} independently of one another denotes hydrogen or a group optionally substituted by one or more identical or different R^{d} and/or R^{e} selected from among C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocyclyl, 4-14 membered heterocyclylalkyl, 5-10 membered heteroaryl and 6-16 membered heteroarylalkyl; and
each R^{d} independently of one another denotes hydrogen or a group optionally substituted by one or more identical or different R^{e} and/or R^{f} selected from among C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocyclyl, 4-14 membered heterocyclylalkyl, 5-10 membered heteroaryl and 6-16 membered heteroarylalkyl;
each R^{e} denotes a suitable group and each independently of one another denote selected from among ═O, —OR^{g}, C₁₋₃haloalkyloxy, —OCF₃, ═S, —SR^{g}, ═NR^{g}, ═NOR^{g}, —NR^{f}R^{f}, halogen, —CF3, —CN, —NC, —OCN, —SCN, —NO, —NO₂, ═N₂, —N₃, —S(O)R^{g}, —S(O)₂R^{g}, —S(O)₂OR^{g}, —S(O)NR^{f}R^{f}, —S(O)₂NR^{f}R^{f}, —OS(O)R^{g}, —OS(O)₂R^{g}, —OS(O)₂OR^{g}, —OS(O)₂NR^{f}R^{f}, —C(O)R^{g}, —C(O)OR^{g}, —C(O)NR^{f}R^{f}, —CN(R^{g})NR^{f}R^{f}, —CN(OH)R^{g}, —C(NOH)NR^{f}R^{f}, —OC(O)R^{g}, —OC(O)OR^{g}, —OC(O)NR^{f}R^{f}, —OCN(R^{g})NR^{f}R^{f}, —N(R^{g})C(O)R^{g}, —N(R^{g})C(S)R^{g}, —N(R^{g})S(O)₂R^{g}, —N(R^{g})C(O)OR^{g}, —N(R^{g})C(O)NR^{f}R^{f}, and —N(R^{g})C(NR^{g})NR^{f}R^{f}, and
each R^{f} independently of one another denotes hydrogen or a group optionally substituted by one or more identical or different R^{g} selected from among C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocyclyl, 4-14 membered heterocyclylalkyl, 5-10 membered heteroaryl and 6-16 membered heteroarylalkyl, and
each R^{g} independently of one another denotes hydrogen, C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocyclyl, 4-14 membered heterocyclylalkyl, 5-10 membered heteroaryl and 6-16 membered heteroarylalkyl, optionally in the form of the tautomers, the racemates, the enantiomers, the diastereomers and the mixtures thereof, and optionally the pharmacologically acceptable salts thereof.

In one aspect the invention relates to compounds of general formula (1), wherein R² denotes a group selected from among C₃₋₁₀cycloalkyl, 3-8 membered heterocyclyl, C₆₋₁₄aryl and 5-10 membered heteroaryl.

In another aspect the invention relates to compounds of general formula (1), wherein R² denotes a group selected from among phenyl and pyridyl.

In one aspect the invention relates to compounds of general formula (1), wherein R³ denotes phenyl.

In one aspect the invention relates to compounds of general formula (1), wherein R⁴ denotes a group selected from among C₁₋₆alkyl, C₆₋₁₄aryl, 3-8 membered heterocyclyl and 5-10 membered heteroaryl.

In one aspect the invention relates to compounds of general formula (1), wherein R⁴ denotes a group selected from among phenyl, isoxazolyl, thienyl and imidazolyl.

In one aspect the invention relates to compounds of general formula (1), or the pharmacologically acceptable salts thereof, for use as pharmaceutical compositions.

In one aspect the invention relates to the use of compounds of general formula (1), or the pharmacologically acceptable salts thereof, for preparing a pharmaceutical composition with an antiproliferative activity.

In one aspect the invention relates to a pharmaceutical preparation, containing as active substance one or more compounds of general formula (1), or the pharmacologically acceptable salts thereof, optionally in combination with conventional excipients and/or carriers.

In one aspect the invention relates to compounds of general formula (1) for preparing a pharmaceutical composition for the treatment and/or prevention of cancer, infections, inflammatory and autoimmune diseases.

In one aspect the invention relates to a pharmaceutical preparation comprising a compound of general formula (1) and at least one other cytostatic or cytotoxic active substance different from formula (1), optionally in the form of the tautomers, the racemates, the enantiomers, the diastereomers and the mixtures thereof, and optionally the pharmacologically acceptable salts thereof.

Definitions

As used herein the following definitions apply, unless stated otherwise.

By alkyl substituents are meant in each case saturated, unsaturated, straight-chain or branched aliphatic hydrocarbon groups (alkyl group) and both saturated alkyl groups and unsaturated alkenyl and alkynyl groups are included. The alkenyl substituents are in each case straight-chain or branched, unsaturated alkyl groups which have at least one double bond. By alkynyl substituents are meant in each case straight-chain or branched, unsaturated alkyl groups which have at least one triple bond.

Heteroalkyl represents straight-chain or branched aliphatic hydrocarbon chains which are interrupted by 1 to 3 heteroatoms, while each of the available carbon and nitrogen atoms in the heteroalkyl chain may optionally each be substituted independently of one another and the heteroatoms are each selected independently of one another from among the group comprising O, N and S (e.g. dimethylaminomethyl, dimethylaminoethyl, dimethylaminopropyl, diethylaminomethyl, diethylaminoethyl, diethylaminopropyl, 2-diisopropylaminoethyl, bis-2-methoxyethylamino, [2-(dimethylamino-ethyl)-ethyl-amino]-methyl, 3-[2-(dimethylamino-ethyl)-ethyl-amino]-propyl, hydroxymethyl, 2-hydroxyethyl, 3-hydroxypropyl, methoxy, ethoxy, propoxy, methoxymethyl, 2-methoxyethyl).

Haloalkyl refers to alkyl groups wherein one or more hydrogen atoms are replaced by halogen atoms. Haloalkyl includes both saturated alkyl groups and unsaturated alkenyl and alkynyl groups, such as for example —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CF═CF₂, —CCl═CH₂, —CBr═CH₂, —CJ=CH₂, —C≡C—CF₃, —CHFCH₂CH₃ and —CHFCH₂CF₃.

Halogen refers to fluorine, chlorine, bromine and/or iodine atoms.

By cycloalkyl is meant a mono- or bicyclic ring, while the ring system may be a saturated ring or an unsaturated, non-aromatic ring, which may optionally also contain double bonds, such as for example cyclopropyl, cyclopropenyl, cyclobutyl, cyclobutenyl, cyclopentyl, cyclopentenyl, cyclohexyl, cyclohexenyl, norbornyl and norbornenyl.

Aryl relates to monocyclic or polycyclic rings with 6-14 carbon atoms such as for example phenyl, naphthyl, anthracene and phenanthrene.

By heteroaryl are meant mono- or polycyclic rings which contain instead of one or more carbon atoms one or more identical or different heteroatoms, such as e.g. nitrogen, sulphur or oxygen atoms. Examples include furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl, pyridyl, pyrimidyl, pyridazinyl, pyrazinyl and triazinyl. Examples of bicyclic heteroaryl groups are indolyl, isoindolyl, benzofuranyl, benzothienyl, benzoxazolyl, benzothiazolyl, benzisoxazolyl, benzisothiazolyl, benzimidazolyl, indazolyl, isoquinolinyl, quinolinyl, quinoxalinyl, cinnolinyl, phthalazinyl, quinazolinyl and benzotriazinyl, indolizinyl, oxazolopyridinyl, imidazopyridinyl, naphthyridinyl, indolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl-N-oxide, pyrimidinyl-N-oxide, pyridazinyl-N-oxide, pyrazinyl-N-oxide, quinolinyl-N-oxide, indolyl-N-oxide, indolinyl-N-oxide, isoquinolyl-N-oxide, quinazolinyl-N-oxide, quinoxalinyl-N-oxide, phthalazinyl-N-oxide, imidazolyl-N-oxide, isoxazolyl-N-oxide, oxazolyl-N-oxide, thiazolyl-N-oxide, indolizinyl-N-oxide, indazolyl-N-oxide, benzothiazolyl-N-oxide, benzimidazolyl-N-oxide, pyrrolyl-N-oxide, oxadiazolyl-N-oxide, thiadiazolyl-N-oxide, triazolyl-N-oxide, tetrazolyl-N-oxide, benzothiopyranyl-S-oxide and benzothiopyranyl-S,S-dioxide.

Heteroarylalkyl comprises a non-cyclic alkyl group wherein a hydrogen atom bound to a carbon atom, usually to a terminal C atom, is replaced by a heteroaryl group.

Heterocyclyl relates to saturated or unsaturated, non-aromatic mono- or polycyclic rings comprising 3-12 carbon atoms, which carry heteroatoms, such as nitrogen, oxygen or sulphur, instead of one or more carbon atoms. Examples of such heterocyclyl groups are tetrahydrofuranyl, pyrrolidinyl, pyrrolinyl, imidazolidinyl, imidazolinyl, pyrazolidinyl, pyrazolinyl, piperidinyl, piperazinyl, indolinyl, isoindolinyl, morpholinyl, thiomorpholinyl, homomorpholinyl, homopiperidinyl, homopiperazinyl, homothiomorpholinyl, thiomorpholinyl-S-oxide, thiomorpholinyl-S,S-dioxide, tetrahydropyranyl, tetrahydrothienyl, homothiomorpholinyl-S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl-S-oxide, tetrahydrothienyl-S,S-dioxide, homothiomorpholinyl-S-oxide, 2-oxa-5-azabicyclo[2,2,1]heptane, 8-oxa-3-aza-bicyclo[3.2.1]octane, 3,8-diaza-bicyclo[3.2.1]octane, 2,5-diaza-bicyclo[2.2.1]heptane, 3,8-diaza-bicyclo[3.2.1]octane, 3,9-diaza-bicyclo[4.2.1]nonane and 2,6-diaza-bicyclo[3.2.2]nonane.

Heterocyclylalkyl relates to a non-cyclic alkyl group wherein a hydrogen atom bound to a carbon atom, usually to a terminal C atom, is replaced by a heterocyclyl group.

The following Examples illustrate the present invention without restricting its scope:

Preparation of the Compounds According to the Invention

The compounds according to the invention may be prepared using the methods of synthesis described hereinafter, where the substituents of the general formulae are as hereinbefore defined.

Chromatography

For medium pressure chromatography (MPLC) silica gel made by Millipore (name: Granula Silica Si-60A 35-70 μm) or C-18 RP-silica gel made by Macherey Nagel (name: Polygoprep 100-50 C18) is used. For high pressure chromatography (HPLC) columns made by Agilent (name: Zorbax SB-C8, 5 μM, 21.2×50 mm) are used.

Mass Spectroscopy/UV Spectrometer:

These data are generated using an HPLC-MS apparatus (high performance liquid chromatography with mass detector) made by Agilent (1100 series).

The apparatus is constructed so that a diode array detector (G1315B made by Agilent) and a mass detector (1100 series LC/MSD Trap/ESI Mode, G1946D; Agilent) are connected in series downstream of the chromatography apparatus (column: Xterra MS C18 2.5 μm, 2.1×50 mm, Messrs. Waters).

HPLC Method 1 (Analytical)

The apparatus is operated with a flow of 0.6 ml/min. For a separation process a gradient is run through within 2 min (start of gradient: 90% water and 10% acetonitrile; end of gradient: 10% water and 90% acetonitrile; in each case 0.1% formic acid is added to the two solvents).

HPLC Method 2 (Analytical)

The apparatus is operated with a flow of 0.6 ml/min. For a separation process a gradient is run through within 3.5 min (start of gradient: 95% water and 5% acetonitrile; end of gradient: 5% water and 95% acetonitrile; in each case 0.1% formic acid is added to the two solvents).

Abbreviations Used

CH₂Cl₂ methylene chloride

DMA dimethylacetamide

DMF N,N-dimethylformamide

DMSO dimethylsulphoxide

Et₂O diethyl ether

EtOAc ethylacetate

h hour(s)

H₂O₂ Hydrogen peroxide

HPLC High pressure liquid chromatography

iPrOH propan-2-ol

iPr₂O Diisopropylether

LiOH lithium hydroxide

M molar

min minute(s)

mL Millilitres

MS mass spectrometry

N normal

NaHCO₃ sodium hydrogen carbonate

NaOH sodium hydroxide

Na₂SO₄ sodium sulphate

Pd(OAc)₂ palladium acetate

RP reversed phase

RT ambient temperature

Rt retention time

tert tertiary

TBTU O-(benzotriazol-1-yl)-N,N,N′,N′-tetramethyluronium tetrafluoroborate

THF tetrahydrofuran

Where the preparation of the starting compounds is not described, they are known, commercially available or may be prepared analogously to known compounds or processes described herein.

### I.1) 4-nitro-2-(arylethenyl)benzenamines—General working method A (GWM A)

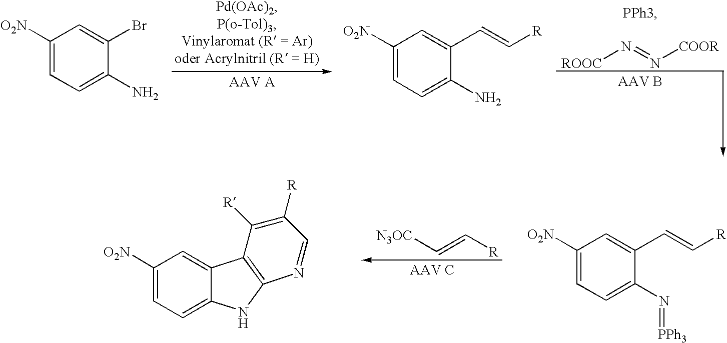

2-bromo-4-nitrobenzenamine (Ando, W.; Tsumaki, H. Synthesis 1982, 10, 263-264), aromatic vinyl compound or acrylonitrile (1.1-2 equivalents), Pd(OAc)₂ (0.01-0.05 equivalents) and tri-o-tolylphosphine (0.03-0.05 equivalents) are refluxed in the presence of a base (triethylamine, cyclohexylmethylamine or N-ethyldiisopropylamine; 1.8 equivalents) under argon in anhydrous DMF, toluene or acetonitrile (2.5-5 mL/g 2-bromo-4-nitrobenzenamine) for 5-12 h with stirring. If the reaction stagnates more Pd(OAc)₂ and tri-o-tolylphosphine may optionally be added. The reaction mixture is freed from the solvent using the rotary evaporator, the residue is taken up in EtOAc (1 L), filtered through Celite, washed with 1 N NaOH and saturated saline solution, dried (Na₂SO₄), filtered and freed from the solvent using the rotary evaporator. The residue is crystallised from toluene, as a result of which the product is obtained as a solid.

The following intermediate compounds are also prepared according to GWM A.

| # | Name | Educt |
|---|---|---|
| I.2 | 4-nitro-2-(2-phenylethenyl)- | styrene |
| | benzenamine | |
| I.3 | 4-nitro-2-[2-(4-pyridinyl)-ethenyl)]- | 4-ethenylpyridine |
| | benzenamine | |
| I.4 | 4-nitro-2-[2-(3-pyridinyl)-ethenyl)]- | 3-ethenylpyridine |
| | benzenamine | |
| I.5 | 4-nitro-2-[2-(4-fluorophenyl)-ethenyl]- | 1-ethenyl-4-fluorobenzene |
| | benzenamine | |
| I.6 | 4-nitro-2-[2-(2-fluorophenyl)-ethenyl]- | 1-ethenyl-2-fluorobenzene |
| | benzenamine | |
| I.7 | 4-nitro-2-[2-(4-methylphenyl)-ethenyl]- | 1-ethenyl-4-methylbenzene |
| | benzenamine | |
| I.8 | 3-(2-amino-5-nitro-phenyl)-acrylonitrile | acrylonitrile |

### II.1) 4-nitro-2-[2-arylethenyl]-N-(triphenylphosphoranylidene)-benzenamine (GWM B)

Diisopropyl or diethyl azodicarboxylate (1.1 equivalents) are added dropwise under argon at 0° C. to a solution of triphenylphosphine (1.1 equivalents) in anhydrous THF (5-15 mL/g amine) and stirred for 1 h. The amine component in anhydrous THF (1-3 mL/g amine) is added and stirred for 2-5 h at RT. The reaction mixture is freed from the solvent using the rotary evaporator and fractionally crystallised from EtOAc.

Furthermore the following intermediate compounds are prepared according to GWM B or analogously thereto.

| # | Name | Educt |
|---|---|---|
| II.2 | 4-nitro-2-[2-phenylethenyl]-N- | I.2 |
| | (triphenylphosphoranylidene)-benzenamine | |
| II.3 | 4-nitro-2-[2-(4-pyridinyl)-ethenyl]-N- | I.3 |
| | (triphenylphosphoranylidene)-benzenamine | |
| II.4 | 4-nitro-2-[2-(3-pyridinyl)-ethenyl]-N | I.4 |
| | (triphenylphosphoranylidene)-benzenamine | |
| II.5 | 4-nitro-2-[2-(4-fluorophenyl)-ethenyl]-N- | I.5 |
| | (triphenylphosphoranylidene)-benzenamine | |
| II.6 | 4-nitro-2-[2-(2-fluorophenyl)-ethenyl]-N- | I.6 |
| | (triphenylphosphoranylidene)-benzenamine | |
| II.7 | 4-nitro-2-[2-(4-methylphenyl)-ethenyl]-N- | I.7 |
| | (triphenylphosphoranylidene)-benzenamine | |
| II.8 | 3-(2-triphenylphosphoranylideneamino-5-nitro-phenyl)- | I.8 |
| | acrylonitrile | |

### Cyclisation to form 3,4-biaryl-α-carboline derivatives (GWM C)

Method 1

Phosphoric acid diphenylester azide (1 equivalent) is added dropwise under argon to a mixture of cinnamic acid derivative or fumaric acid derivative and triethylamine (1 equivalent) in anhydrous toluene (10-50 mL/g cinnamic acid derivative) and stirred for 12 h at RT. Then the mixture is heated to boiling temperature and stirred for 3 h. The iminophosphorane (0.8 equivalents) is added thereto in solid form, the mixture is stirred for another 4 h and then at this temperature air is piped through the reaction mixture for 12 h. The reaction mixture is freed from the solvent using the rotary evaporator, taken up in CH₂Cl₂, washed with saturated ammonium chloride solution and saturated saline solution, dried (Na₂SO₄), filtered through silica gel and highly concentrated by evaporation using the rotary evaporator. The residue is fractionally crystallised from EtOAc at −4° C. or purified by chromatography.

Method 2

At 5° C. a mixture of sodium azide (1 equivalent) and tetrabutylammonium chloride (0.1 equivalents) in water (15-25 mL/g sodium azide) is added dropwise to a solution of the substituted cinnamic acid chloride in anhydrous toluene (15-30 mL/g cinnamic acid chloride) and stirred for 40-90 min at 15-40° C. The organic phase is separated off, dried (Na₂SO₄), filtered and stirred at 100° C. until no more gas is given off. The iminophosphorane (0.8 equivalents) is added in solid form, the mixture is stirred for another 4 h and then at this temperature air is piped through the reaction mixture for 12 hours. The reaction mixture is freed from the solvent using the rotary evaporator, taken up in CH₂Cl₂, washed with saturated ammonium chloride solution and saturated saline solution, dried (Na₂SO₄), filtered through silica gel and highly concentrated by evaporation using the rotary evaporator. The residue is fractionally crystallised from EtOAc at −4° C. or purified by chromatography.

The following cyclisation reactions are carried out according to GWM C.

| # | structure | cinnamic acid derivative | educt | method |
|---|---|---|---|---|
| III.1 | 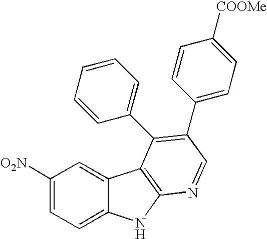 | 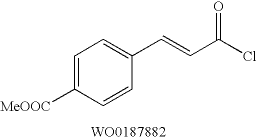 | II.1 | 2 |
| III.2 | 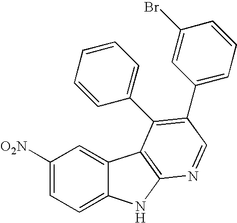 | 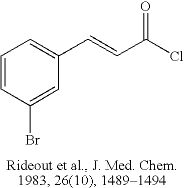 | II.1 | 2 |
| III.3 | 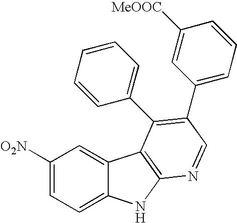 | 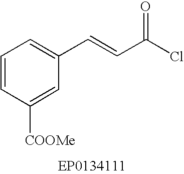 | II.1 | 2 |
| III.4 | 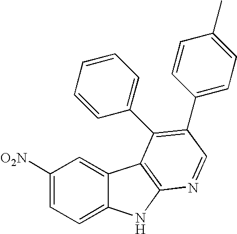 | 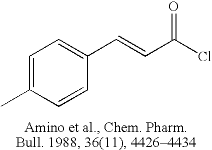 | II.1 | 2 |
| III.5 | 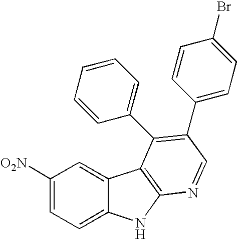 | 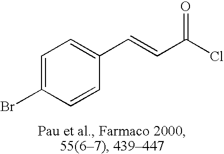 | II.1 | 2 |
| III.6 | 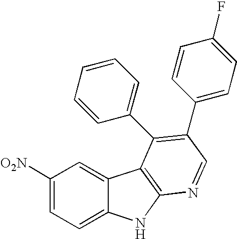 | 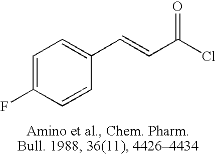 | II.1 | 2 |
| III.7 | 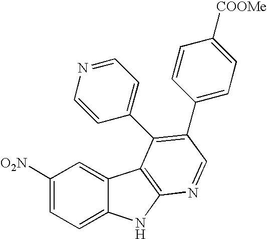 | 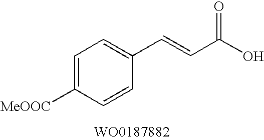 | II.2 | 2 |
| III.8 | 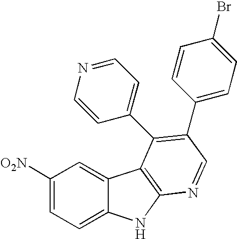 | 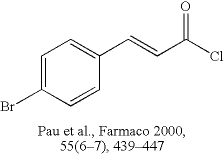 | II.2 | 2 |
| III.9 | 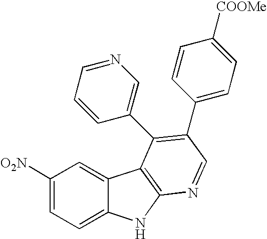 | 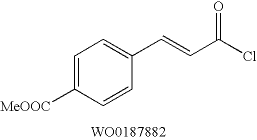 | II.3 | 2 |
| III.10 | 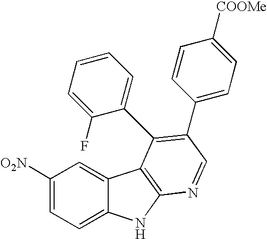 | 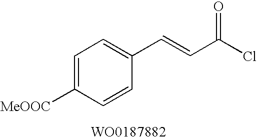 | II.5 | 2 |
| III.11 | 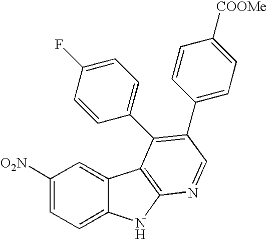 | 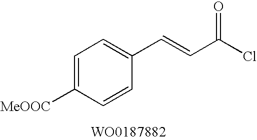 | II.4 | 2 |
| III.12 | 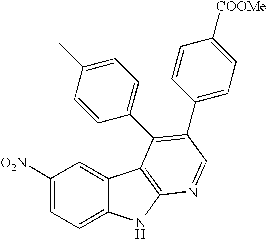 | 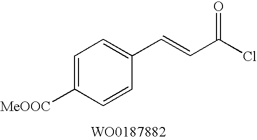 | II.6 | 2 |
| III.13 | 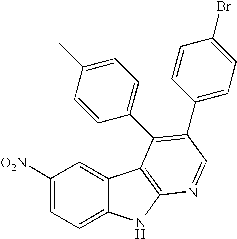 | 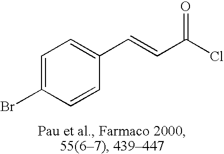 | II.6 | 2 |
| III.14 | 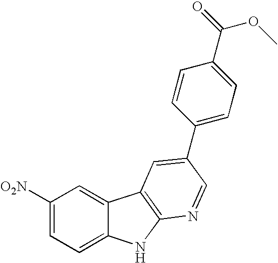 | 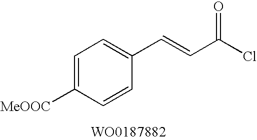 | II.8 | 2 |
| III.15 | 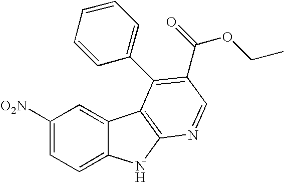 | 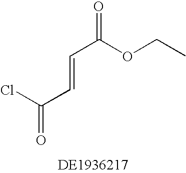 | II.8 | 2 |

### Ester Cleaving at Carboline Derivatives (GWM D)

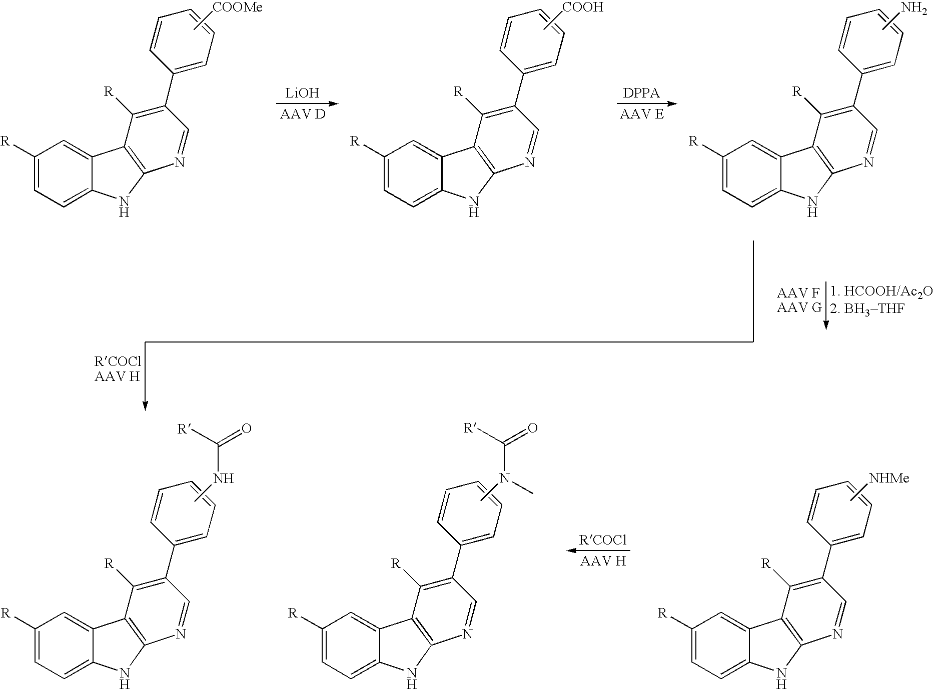

1 N aqueous LiOH solution (10 equivalents) is added at RT to a solution of the carboline ester in DMF, THF, methanol or a mixture of these solvents (10-60 mL/g ester) and the mixture is stirred for 12-48 h. The mixture is optionally diluted with 1 N LiOH, washed with Et₂O or EtOAc, the aqueous phase is acidified with 2 N HCl and the carboxylic acid precipitated is obtained by extraction or filtration.

The following intermediate compounds are prepared according to GWM D or analogously thereto.

| # | structure | educt |
|---|---|---|
| IV.1 | 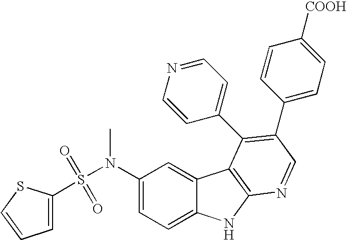 | 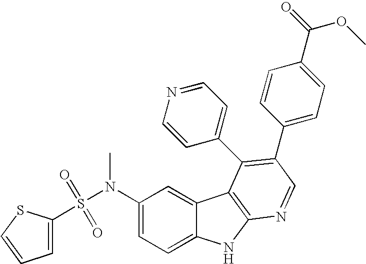 |
| IV.2 | 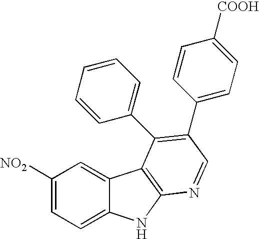 | III.1 |
| IV.3 | 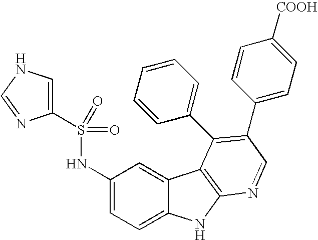 | 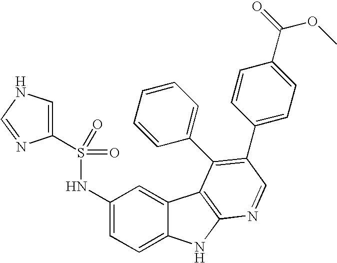 |
| IV.4 | 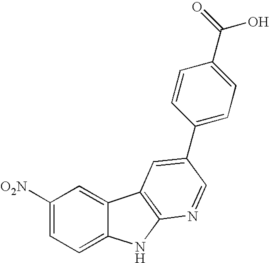 | III.14 |
| IV.5 | 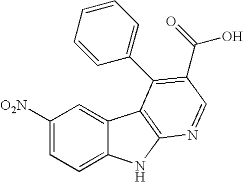 | III.15 |

### Acid Decomposition (GWM E)

Triethylamine and phosphoric acid diphenylester azide (1.5 equivalents of each) are added to a suspension or solution of the carbolinecarboxylic acid in DMF (15-30 mL/g educt) and stirred for 12-24 h at RT. Water is added (0.6 mL/mL DMF) and the mixture is stirred for 1-5 h at 100° C. After the reaction has ended it is diluted with water and the product is obtained by extraction or filtration.

The following intermediate compounds are prepared according to GWM E or analogously thereto.

| # | structure | educt |
|---|---|---|
| V.1 | 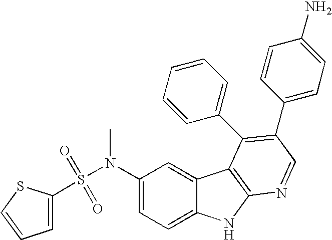 | 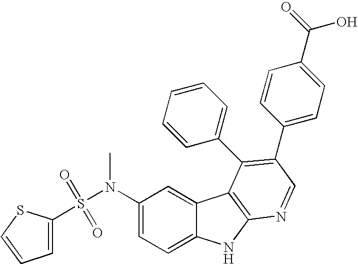 |
| V.2 | 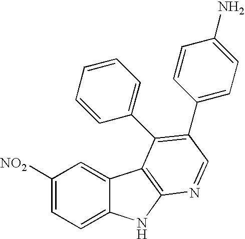 | IV.2 |
| V.3 | 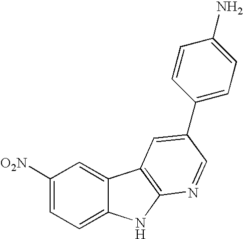 | IV.4 |
| V.4 | 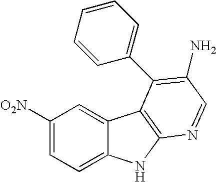 | IV.5 |

### Formylation of Carbolinamines (GWM F)

Formic acid (10 mL/g educt) and acetic anhydride (2-5 equivalents) are stirred for 1-5 h at 10-50° C. and diluted with anhydrous THF (20-30 mL/1 g educt). Then the amine is added batchwise over a period of 10 min and the mixture is stirred for 1 h at RT. The product is obtained either by precipitation with tert-butylmethylether or by extraction and optionally purified by chromatography.

The following intermediate compounds are prepared according to GWM F.

| # | structure | educt |
|---|---|---|
| VI.1 | 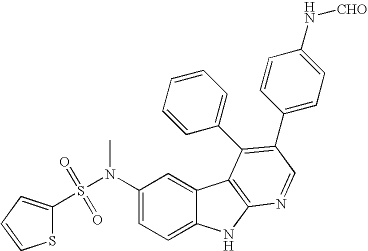 | V.1 |
| VI.2 | 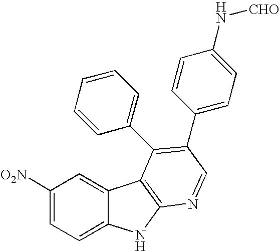 | V.2 |
| VI.3 | 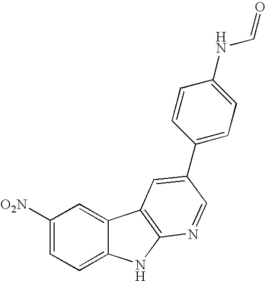 | V.4 |
| VI.4 | 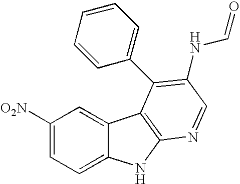 | V.5 |

### Reduction to N-methylcarbolinamines (GWM G)

Borane-dimethylsulphide complex or borane-THF complex (2-20 equivalents) is added dropwise at RT to a solution of the starting compound in anhydrous THF (10-50 mL) and stirred for 2-10 h at RT. Then additional borane complex is optionally added dropwise and the mixture is stirred overnight at RT.

Working Up According to Method 1

Tetramethylethylenediamine (10-50 equivalents) is added and the mixture is stirred for 48 h at RT. Dilute NaHCO₃ solution is added, the aqueous phase is exhaustively extracted with EtOAc, and the combined organic phases are washed with NaHCO₃, water and saturated saline solution, dried (MgSO₄), filtered and freed from the solvent using the rotary evaporator. The residue is optionally purified by chromatography.

Working Up According to Method 2

The pH is adjusted to about 1 with 2 N HCl and the mixture is stirred for 2 h at RT, then neutralised with 1 N NaOH, the product is isolated by extraction with CH₂Cl₂ and optionally purified by chromatography.

The following intermediate compounds are prepared according to GWM G.

| # | structure | educt |
|---|---|---|
| VII.1 | 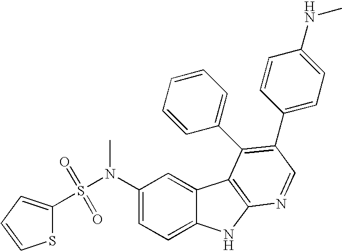 | VI.1 |
| VII.2 | 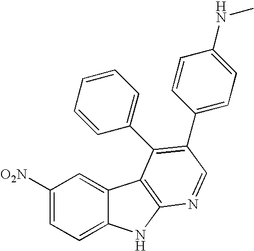 | VI.2 |
| VII.3 | 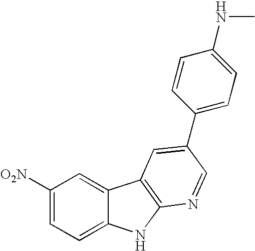 | VI.3 |
| VII.4 | 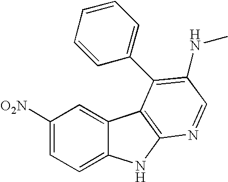 | VI.4 |

### Amide Formation (GWM H)

Method 1 Starting from Acid Chlorides or Anhydrides

The acid chloride or anhydride (1.1-5 equivalents), in substance or as a solution in anhydrous CH₂Cl₂, and then pyridine (3-50 equivalents) are added successively to a solution of the primary or secondary amine in anhydrous CH₂Cl₂ (10-100 mL/g educt) and stirred for 1-12 h at RT. The reaction solution is diluted with CH₂Cl₂, with water, saturated ammonium chloride solution, saturated NaHCO₃ solution and saturated saline solution, dried (Na₂SO₄), filtered, freed from the solvent using the rotary evaporator and optionally purified by chromatography.

Method 2 Starting from Carboxylic Acids Using TBTU

A solution of amine, carboxylic acid (1 equivalent), TBTU (1.2 equivalents) and a base (triethylamine, pyridine or N-ethyldiisopropylamine; 1-5 equivalents) in anhydrous DMF (10-20 mL/g amine) are stirred for 2-15 h at RT. If necessary, more carboxylic acid and TBTU are metered in. The reaction solution is freed from the solvent using the rotary evaporator, the residue is taken up in CH₂Cl₂, washed with water, saturated ammonium chloride solution, saturated NaHCO₃ solution and saturated saline solution, dried (Na₂SO₄), filtered, freed from the solvent using the rotary evaporator and optionally purified by chromatography.

The following intermediate compounds are prepared according to GWM H.

| # | structure | educt |
|---|---|---|
| VIII.1 | 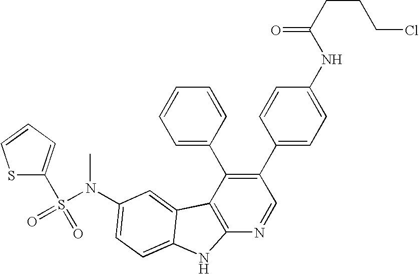 | V.1 |
| VIII.2 | 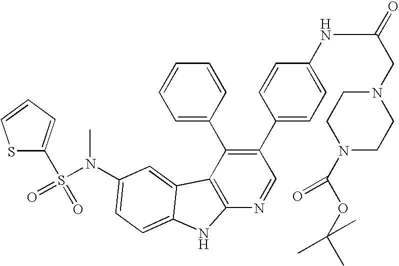 | V.1 |
| VIII.3 | 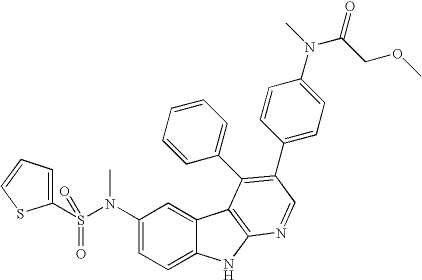 | V.1 |

The preparation of sulphonamides optionally substituted at the nitrogen atom is carried out analogously to GWM H or GWM J.

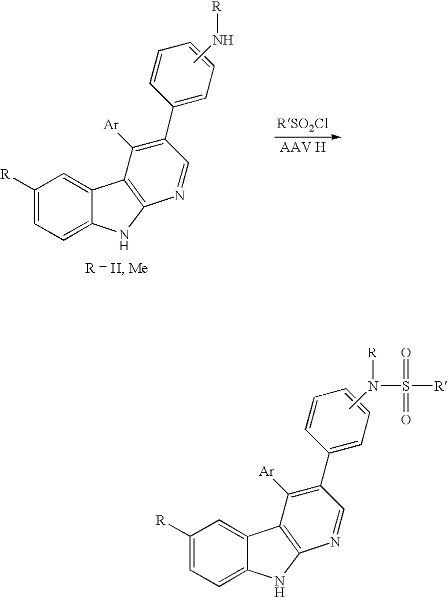

| # | structure | educt |
|---|---|---|
| IX.1 | 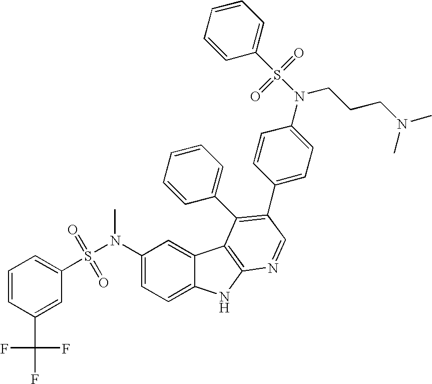 | 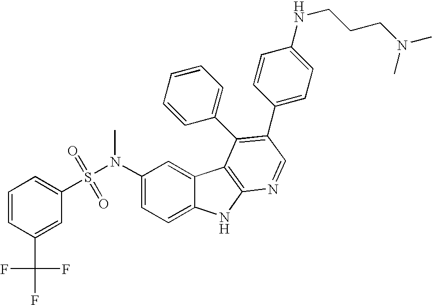 |
| IX.2 | 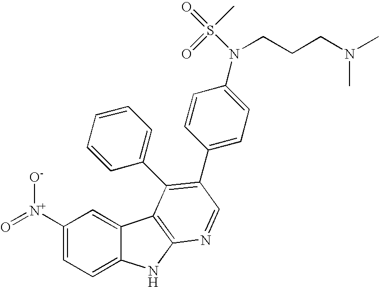 | 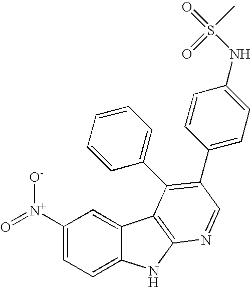 |
| IX.3 | 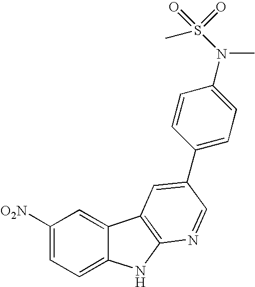 | VII.3 |
| IX.4 | 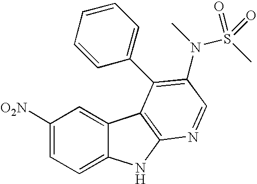 | VII.4 |

### Reduction of Nitrocarboline Derivatives to the Corresponding Amines (GWM I)

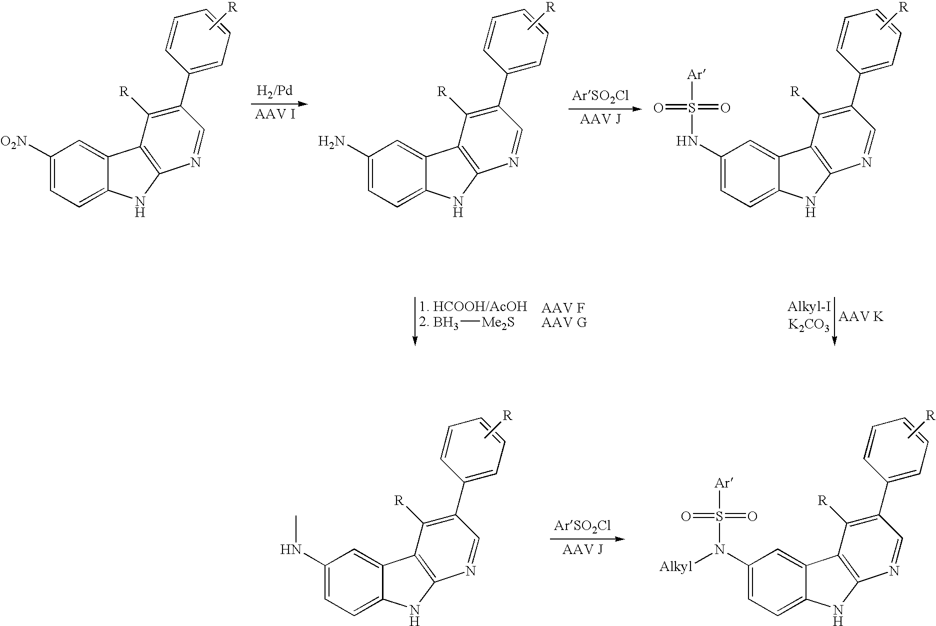

A mixture of nitro compound and palladium on activated charcoal (5% or 10%) or Raney nickel (5-25 mg/g nitro compound) in methanol, THF, 50% methanol in THF or DMF is hydrogenated under a hydrogen pressure of 3-10 bar at a temperature between 15-60° C. over a period of 3-48 h. The reaction mixture is degassed with nitrogen and the catalyst is filtered off through Celite. The solvent is eliminated using the rotary evaporator and the residue is optionally purified by chromatography.

The following intermediate compounds are prepared according to GWM I.

| # | structure | educt |
|---|---|---|
| X.1 | 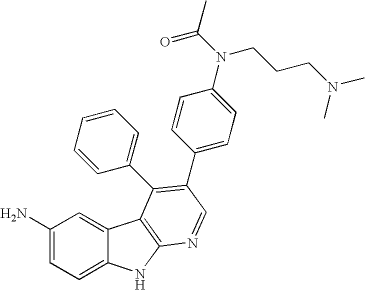 | 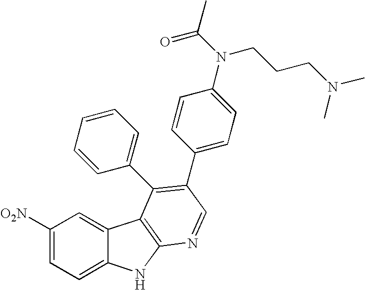 |
| X.2 | 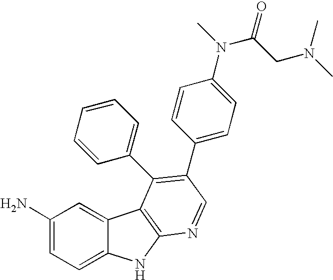 | 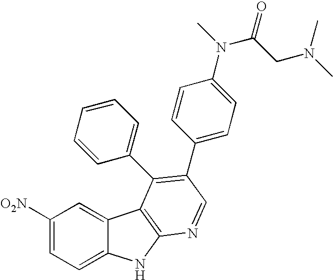 |
| X.3 | 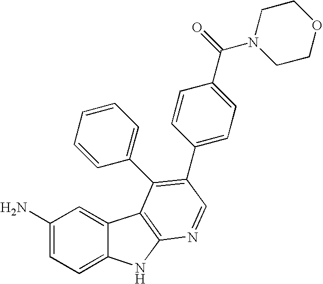 | 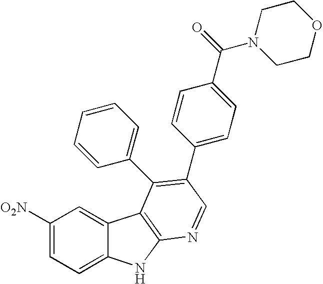 |
| X.4 | 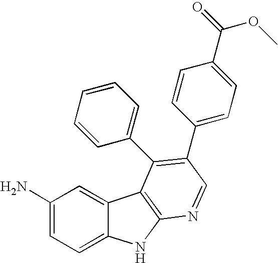 | III.1 |
| X.5 | 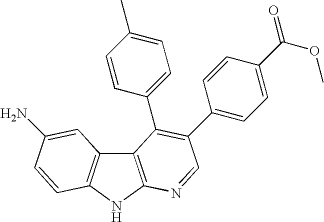 | III.12 |
| X.6 | 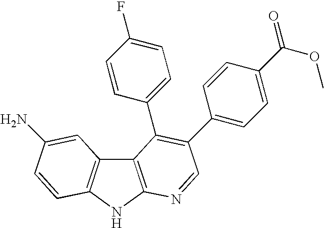 | III.11 |
| X.7 | 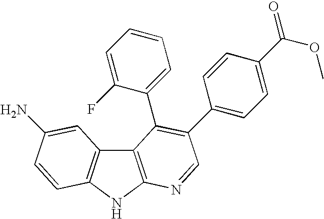 | III.10 |
| X.8 | 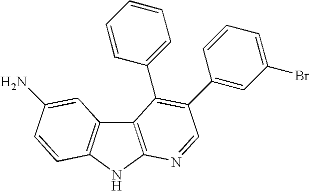 | 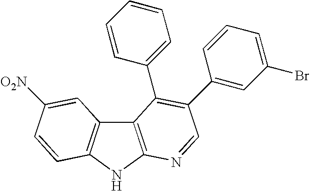 |
| X.9 | 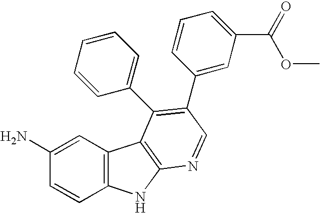 | 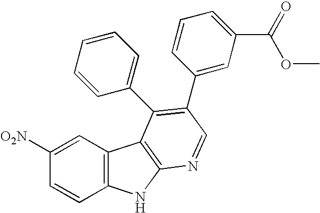 |
| X.10 | 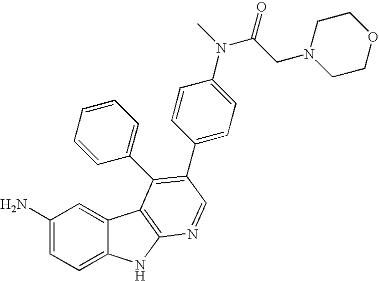 | 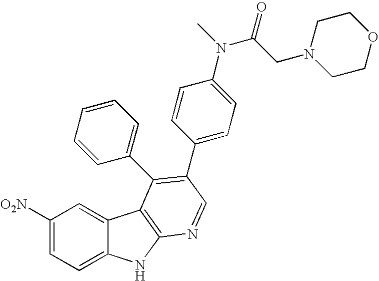 |
| X.11 | 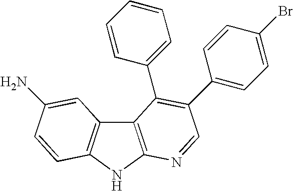 | III.5 |
| X.12 | 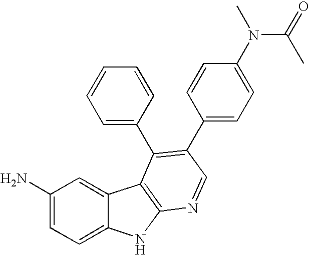 | 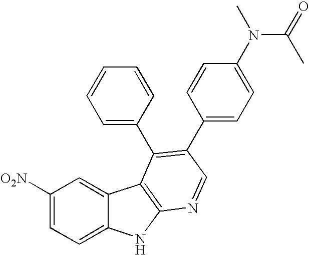 |
| X.13 | 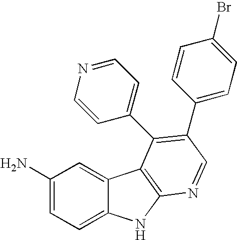 | III.8 |
| X.14 | 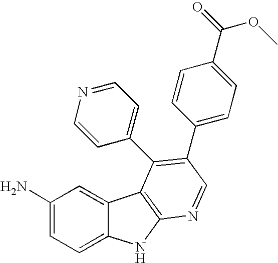 | 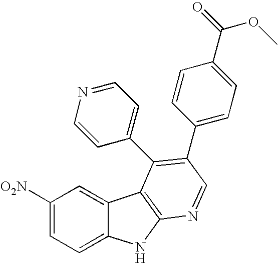 |
| X.15 | 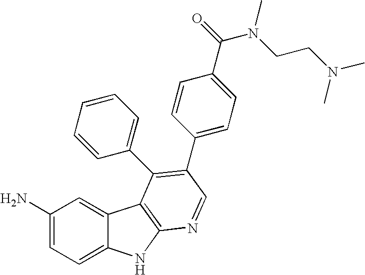 | 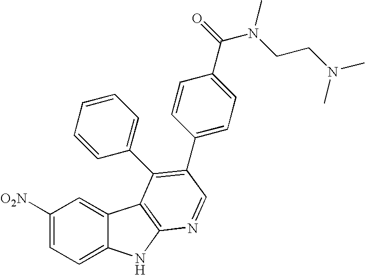 |
| X.16 | 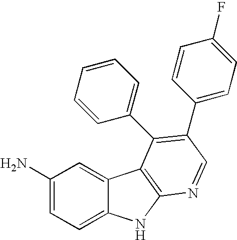 | 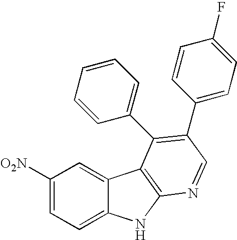 |
| X.17 | 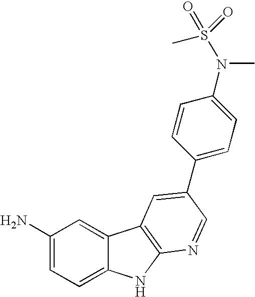 | IX.3 |
| X.18 | 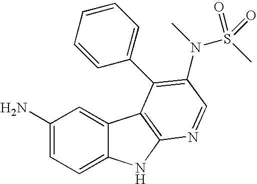 | IX.4 |

### Sulphonamide Formation (GWM F)

Anhydrous pyridine, triethylamine or N-ethyldiisopropylamine (3-15 equivalents) is added at 0° C. under argon to a mixture of amine and sulphonic acid chloride (1-5 equivalents) in anhydrous CH₂Cl₂ (10-50 mL/g amine) and stirred for 2 to 24 h at RT. The reaction mixture is washed with aqueous ammonium chloride solution, saturated NaHCO₃ solution and saturated saline solution, dried (Na₂SO₄), filtered and freed from the solvent using the rotary evaporator. The crude product is purified by crystallisation or by column chromatography.

The following intermediate compounds are prepared according to GWM J.

| # | structure | educt |
|---|---|---|
| XI.1 | 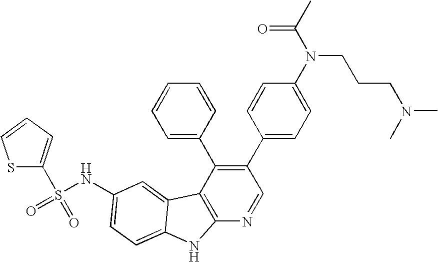 | X.1 |
| XI.2 | 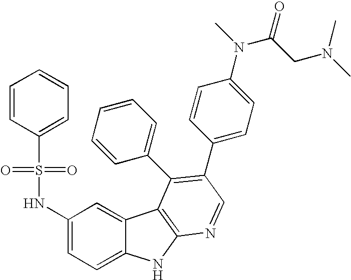 | 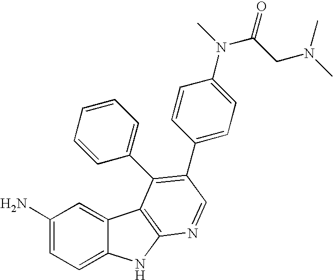 |
| XI.3 | 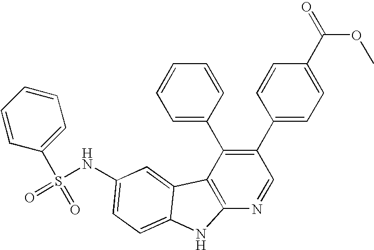 | 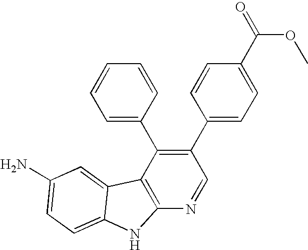 |
| XI.4 | 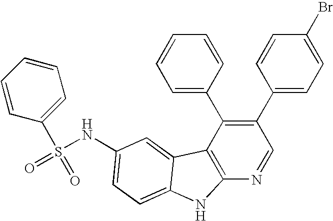 | 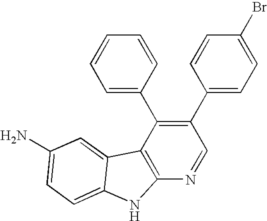 |
| XI.5 | 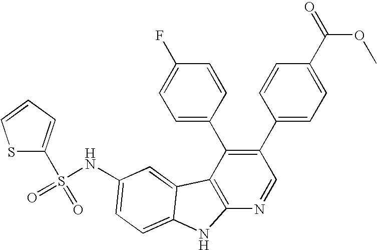 | 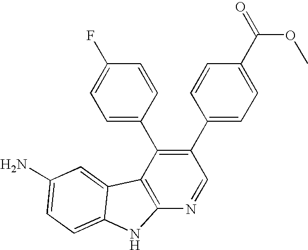 |
| XI.6 | 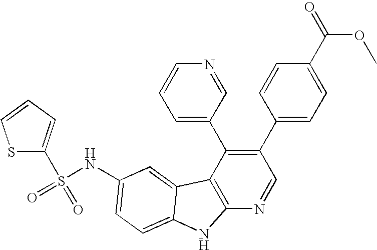 | 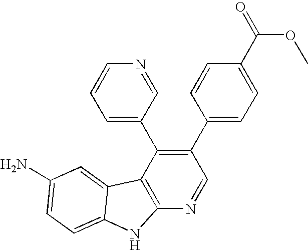 |
| XI.7 | 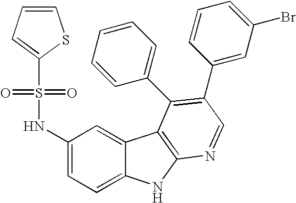 | X.8 |
| XI.8 | 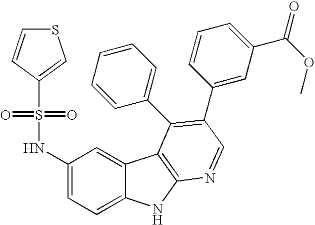 | 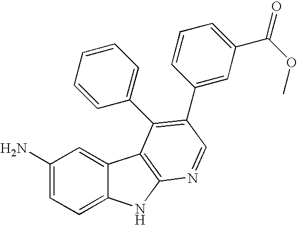 |
| XI.9 | 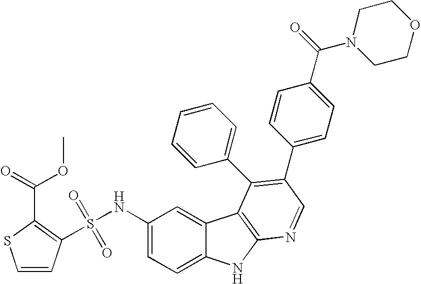 | 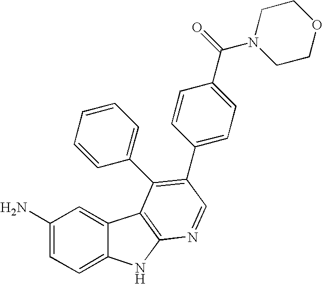 |
| XI.10 | 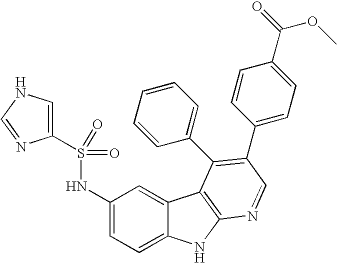 | 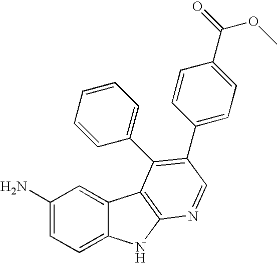 |

The introduction of a methyl group into carbolin-6-amines is carried out by formylation and subsequent reduction according to GWM F and G.

The following intermediate compounds are prepared by formylation or subsequent reduction according to GWM F and G.

| # | structure | educt |
|---|---|---|
| XII.1 | 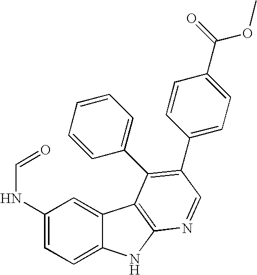 | 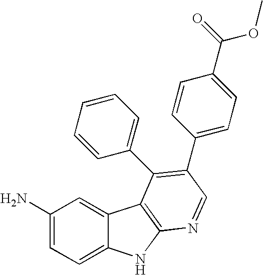 |
| XII.2 | 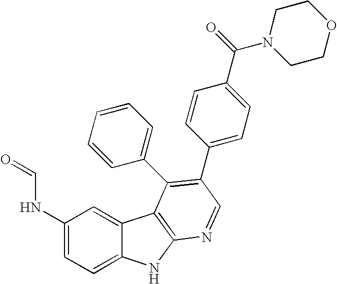 | 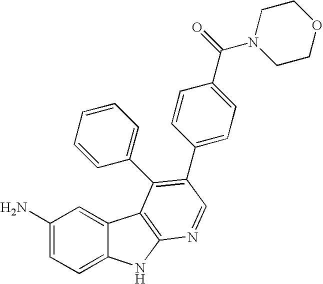 |
| XII.3 | 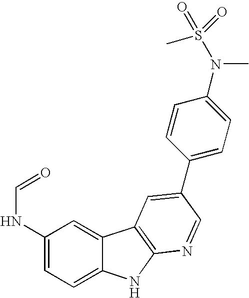 | X.17 |
| XII.4 | 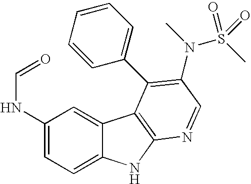 | X.18 |
| XIII.1 | 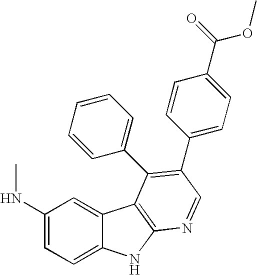 | XII.1 |
| XIII.2 | 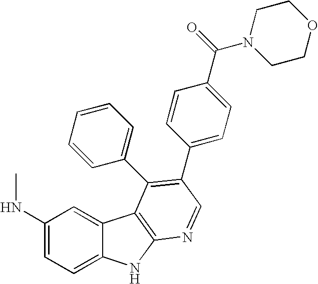 | XII.2 |
| XIII.3 | 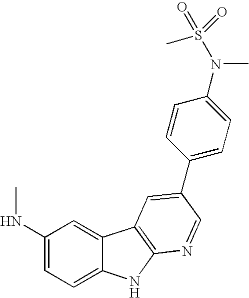 | XII.3 |
| XIII.4 | 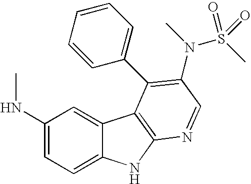 | XII.4 |

### N-Alkylation of Sulphonamides (GWM K)

Freshly ground potassium carbonate (anhydrous, 1-4 equivalents) and the alkylating agent (methyl iodide or dimethyl sulphate or ethyl iodide; 1.1-1.5 equivalents, as 10% solution in DMF) are added successively at 0° C. to a solution of the sulphonamide in anhydrous DMF (10-30 mL/g educt)and stirred for 12-36 h at RT. Concentrated ammonia solution is added, the mixture is diluted with CH₂Cl₂, the aqueous phase is extracted quantitatively with CH₂Cl₂, the combined organic phases are washed with saturated ammonium chloride solution, saturated NaHCO₃ solution and saturated saline solution, dried (Na₂SO₄), filtered and the mixture is freed from solvent using the rotary evaporator. The crude product is purified by column chromatography.

The following compounds are prepared according to GWM H.

| # | structure | educt |
|---|---|---|
| XIV.1 | 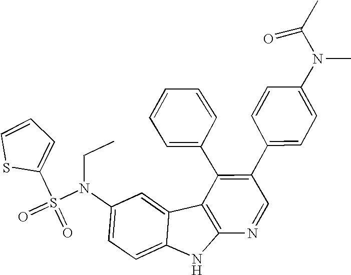 | 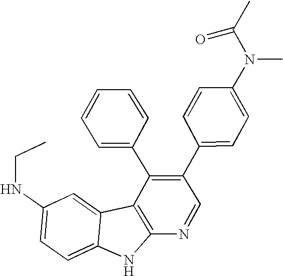 |
| XIV.2 | 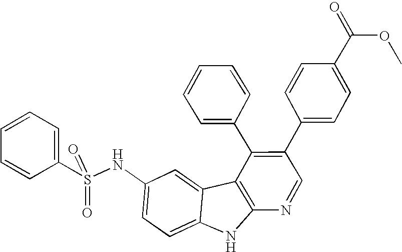 | X.4 |
| XIV.3 | 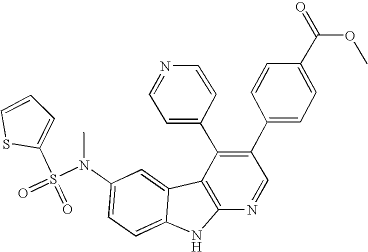 | 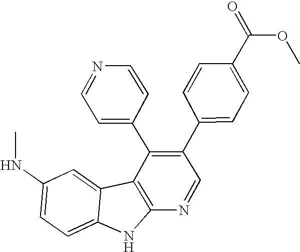 |
| XIV.4 | 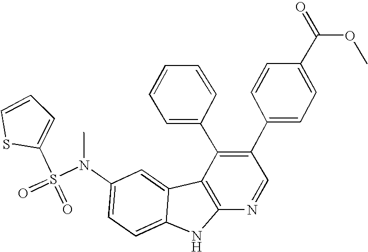 | 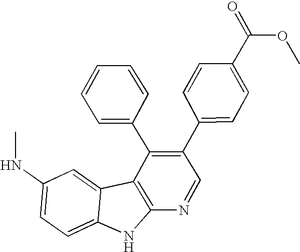 |
| XIV.5 | 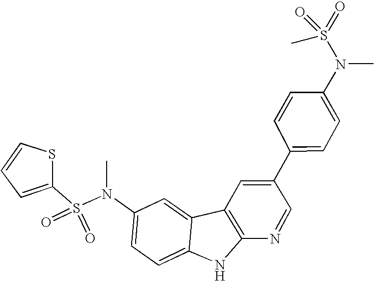 | XIII.3 |
| XIV.6 | 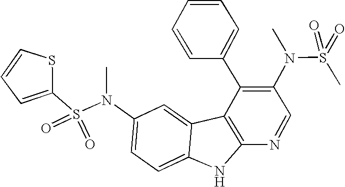 | XIII.4 |

### Reaction of carboline-ω-halocarboxylic acid-amides and carboline-ω-halosulphonic acid amides with secondary amines (GWM L)

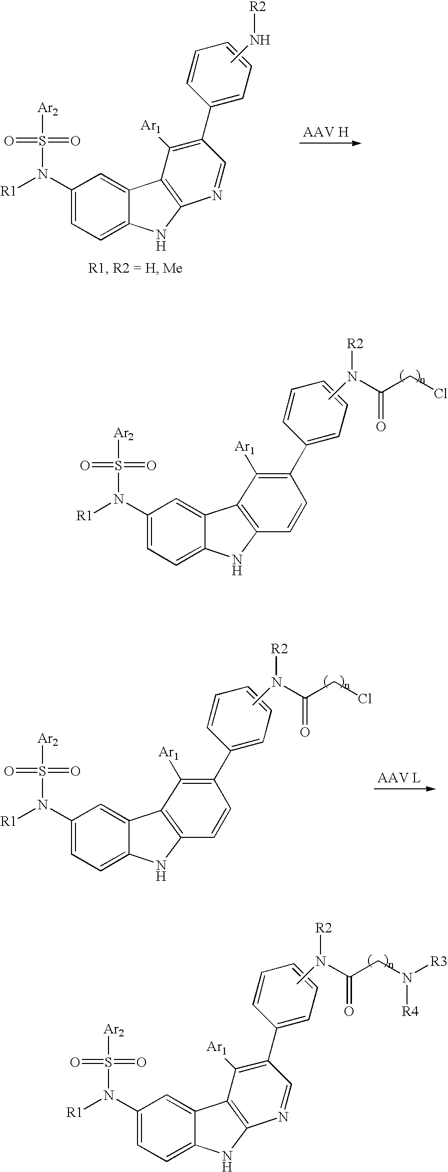

A mixture of educt (20-200 mg; prepared according to GWM H/Method 1 for carboxylic acid amides or GWM J for sulphonamides) and secondary amine (1.5-10 equivalents) are stirred in N-methylpyrrolidinone, DMF or DMA (10-50 μL/mg educt) in the microwave reactor for 5-20 min at 150° C. The reaction mixture is purified by preparative HPLC and the eluate is freed from the solvent by freeze-drying.

The following compounds are prepared according to GWM H.

| # | structure | educt |
|---|---|---|
| XV.1 | 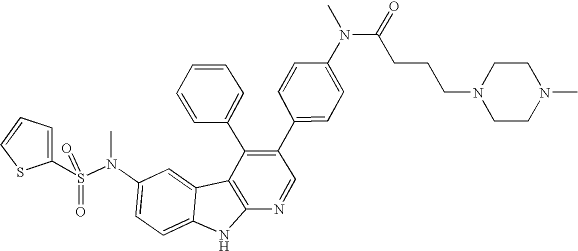 | 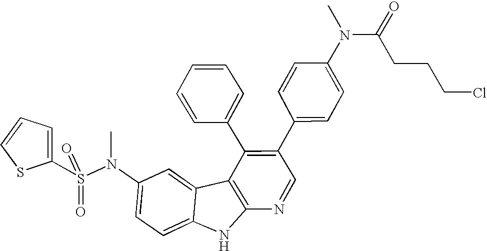 |
| XV.2 | 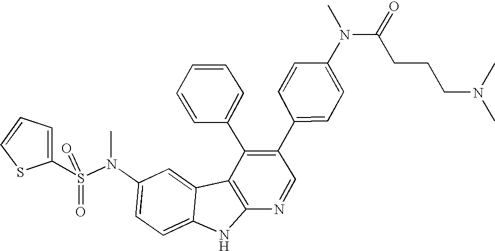 | 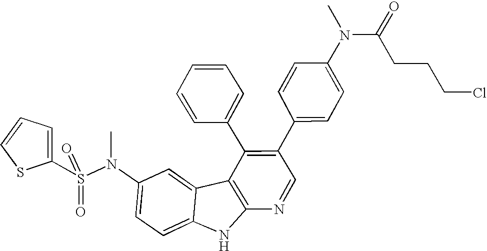 |
| XV.3 | 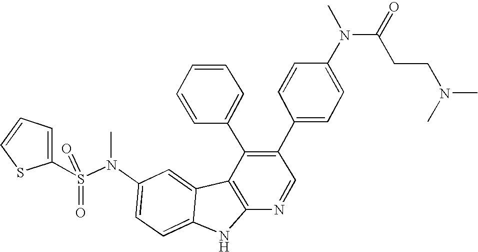 | 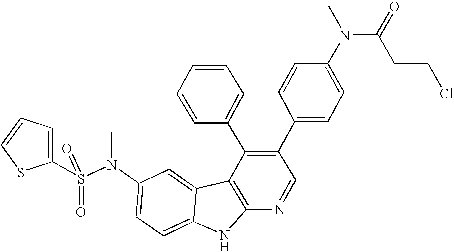 |
| XV.4 | 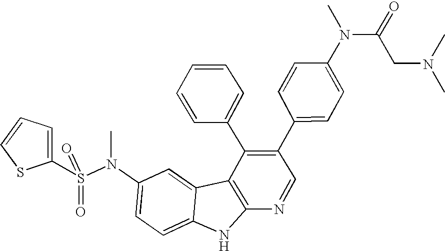 | 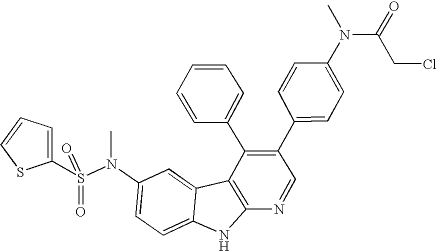 |
| XV.5 | 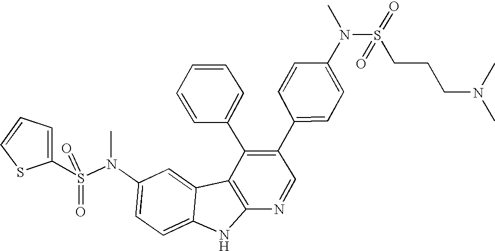 | 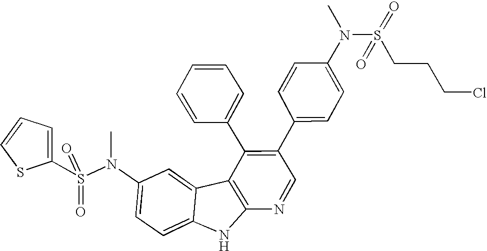 |
| XV.6 | 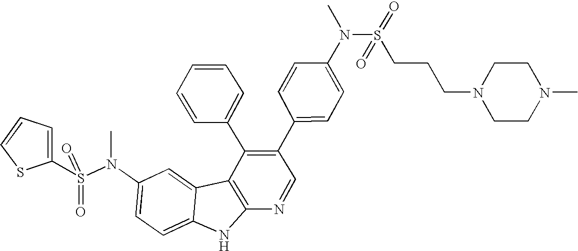 | 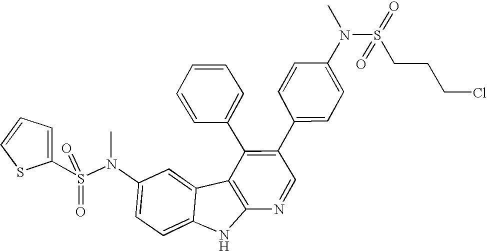 |

### Reduction of Carbolinecarboxylic Acid Amides to Amines (GWM M)

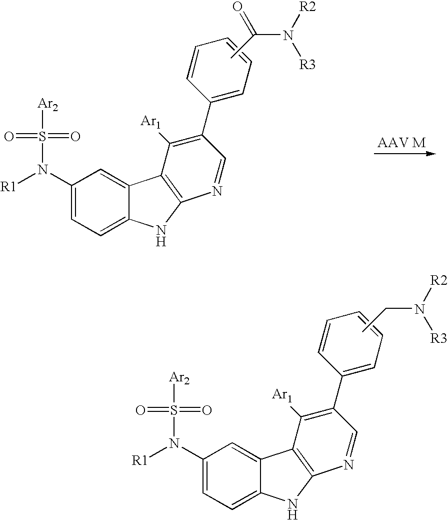

Lithium aluminium hydride (3-7 equivalents) is added at 0° C. to a solution of the carboxylic acid amide in anhydrous THF (10-50 mL/g educt) and stirred for 2-24 h at RT. If the reaction stagnates stirring is continued at boiling temperature. The mixture is hydrolysed with water in THF (50%) until a precipitate is formed, which is separated off by filtration and decocted with methanol. The combined organic phases are freed from the solvent using the rotary evaporator, the residue is purified by preparative HPLC and the eluate is freed from the solvent by freeze-drying.

The following compounds are prepared according to GWM M.

| # | structure | educt |
|---|---|---|
| XVI.1 | 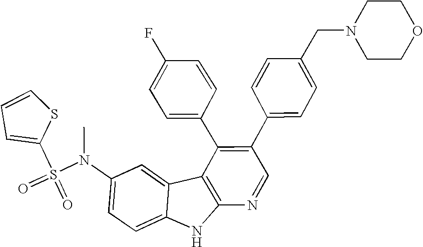 | 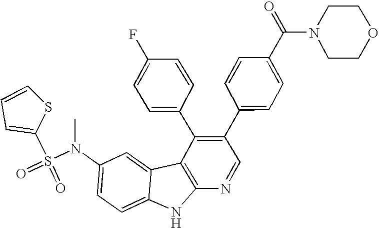 |
| XVI.3 | 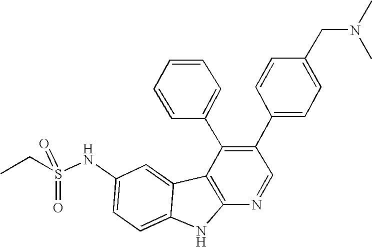 | 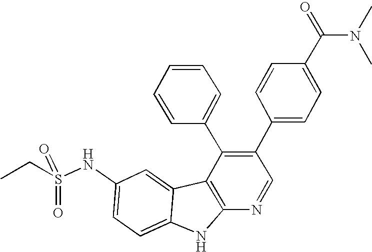 |

### EXAMPLES 1-173

The substances are prepared according to GWM A-M.

| # | structure | tᵣₑₜ (min) | mass [M + H] |
|---|---|---|---|
| 1 | 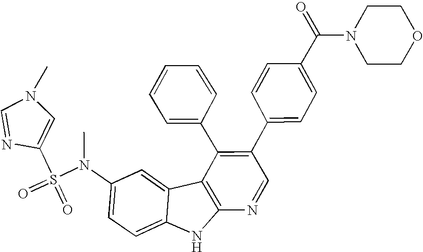 | 2.97 | 607 |
| 2 | 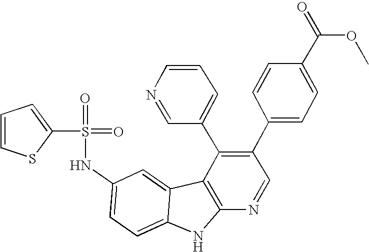 | 3.12 | 541 |
| 3 | 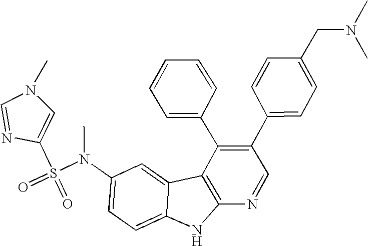 | 2.67 | 551 |
| 4 | 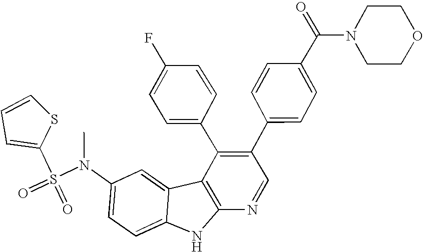 | 3.25 | 627 |
| 5 | 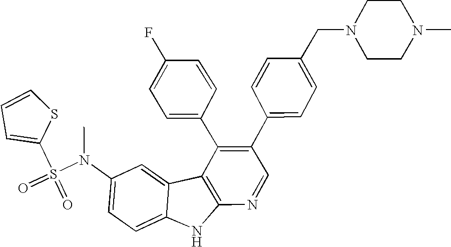 | 2.91 | 626 |
| 6 | 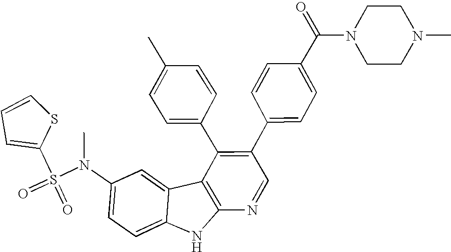 | 2.81 | 636 |
| 7 | 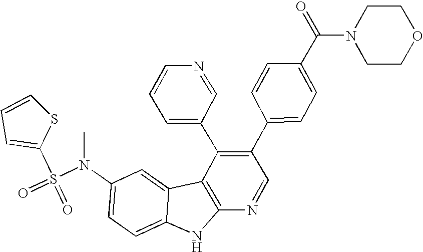 | 2.97 | 610 |
| 8 | 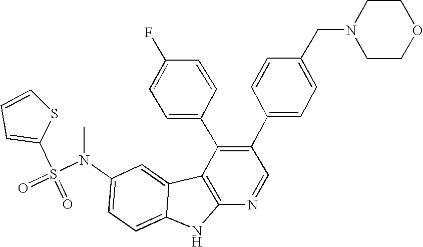 | 2.90 | 613 |
| 9 | 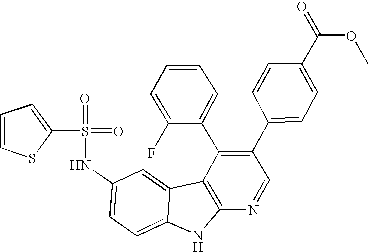 | 3.31 | 558 |
| 10 | 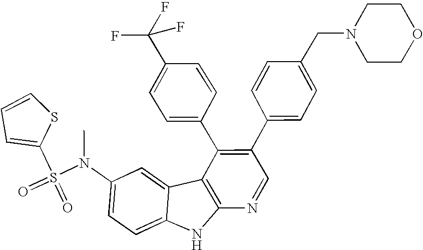 | 2.95 | 663 |
| 11 | 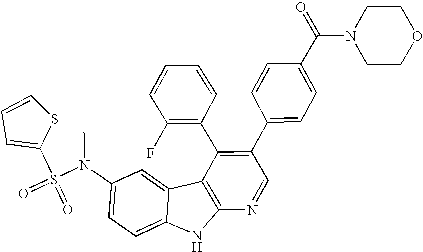 | 3.21 | 627 |
| 12 | 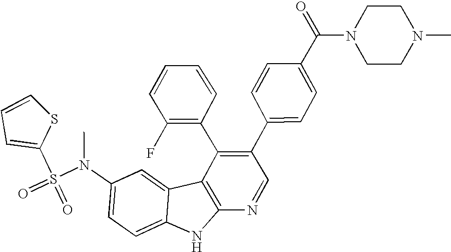 | 2.87 | 640 |
| 13 | 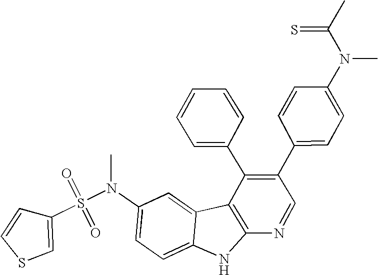 | 3.47 | 583 |
| 14 | 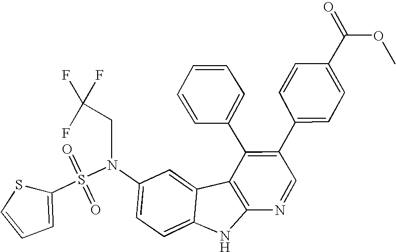 | 3.64 | 622 |
| 15 | 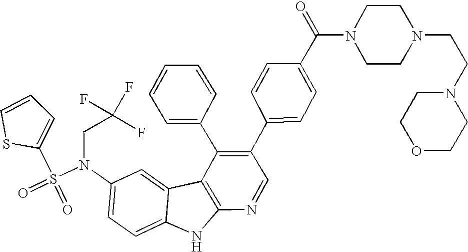 | 2.78 | 789 |
| 16 | 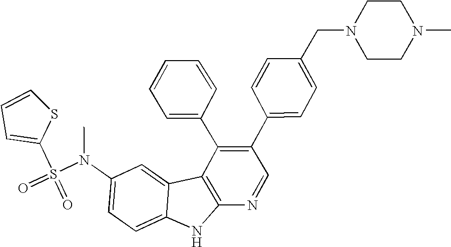 | 2.72 | 608 |
| 17 | 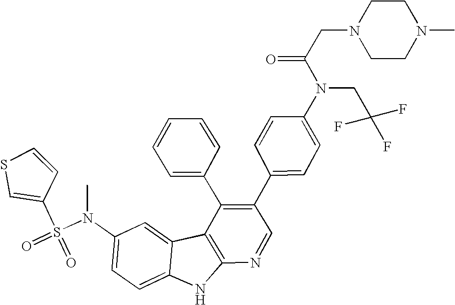 | 2.89 | 733 |
| 18 | 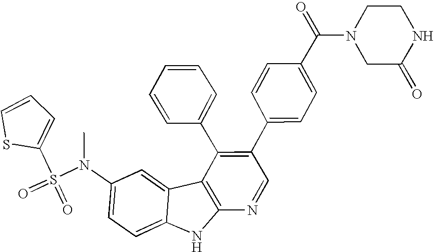 | 3.65 | 622 |
| 19 | 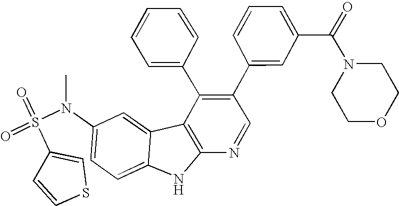 | 3.20 | 609 |
| 20 | 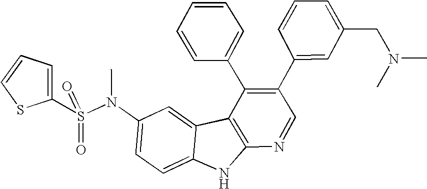 | 2.83 | 553 |
| 21 | 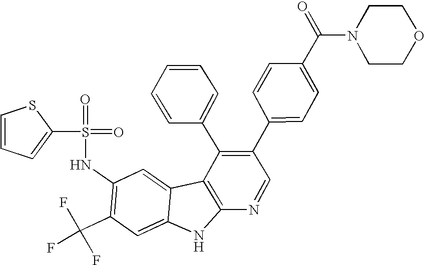 | 3.21 | 663 |
| 22 | 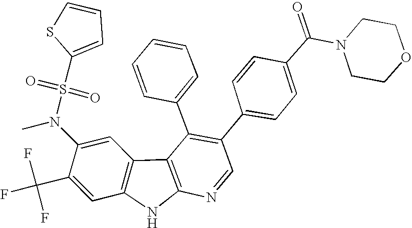 | 3.32 | 677 |
| 23 | 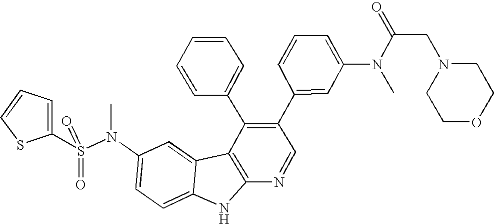 | 2.85 | 652 |
| 24 | 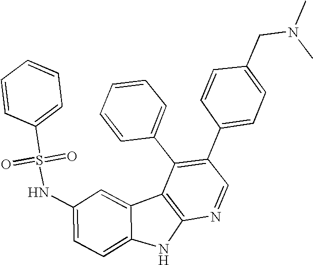 | | 533 |
| 25 | 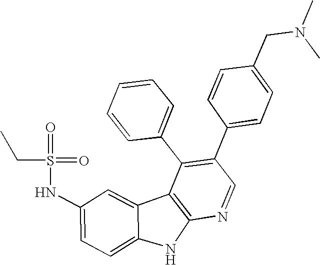 | 2.84 | 485 |
| 26 | 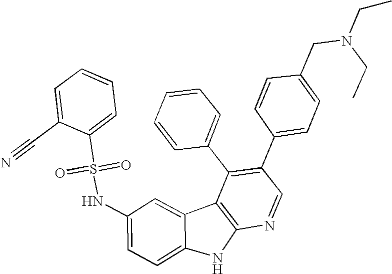 | 3.04 | 586 |
| 27 | 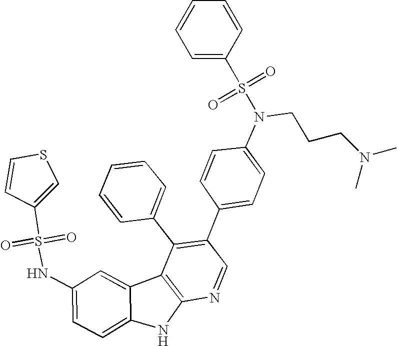 | | 722 |
| 28 | 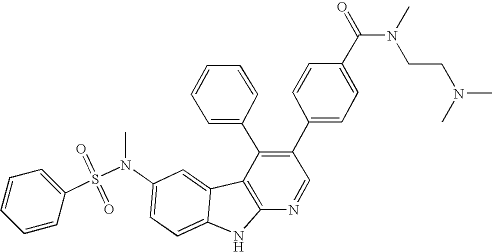 | 3.30 | 618 |
| 29 | 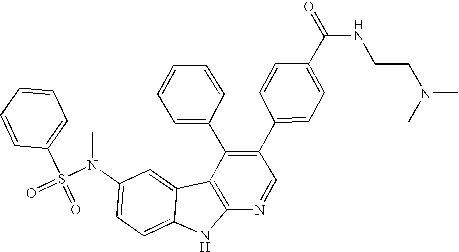 | 3.30 | 604 |
| 30 | 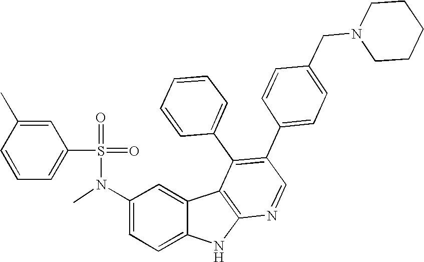 | 3.43 | 601 |
| 31 | 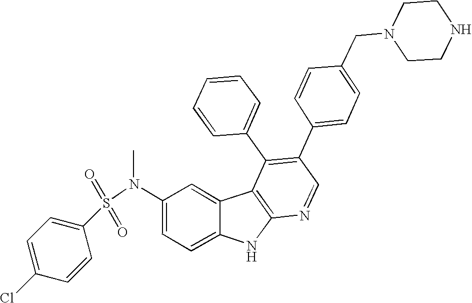 | | 623 |
| 32 | 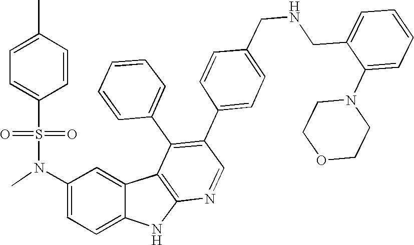 | | 708 |
| 33 | 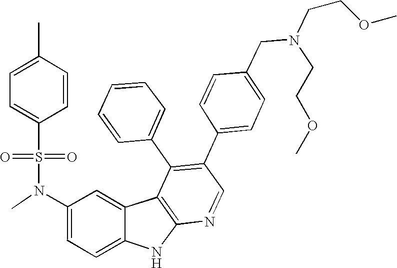 | 3.45 | 649 |
| 34 | 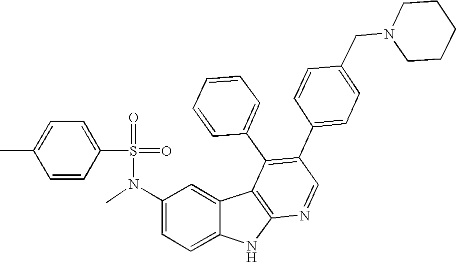 | 3.47 | 601 |
| 35 | 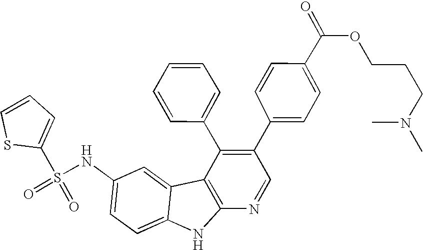 | 3.18 | 611 |
| 36 | 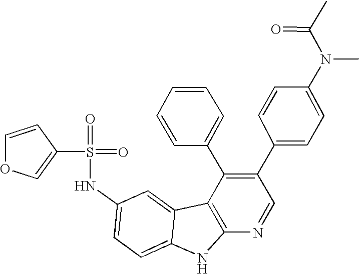 | 3.75 | 537 |
| 37 | 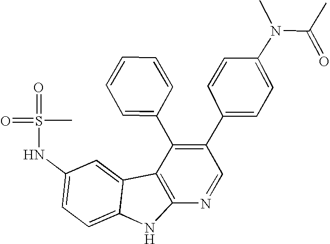 | 3.49 | 485 |
| 38 | 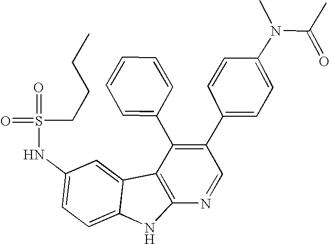 | 3.86 | 527 |
| 39 | 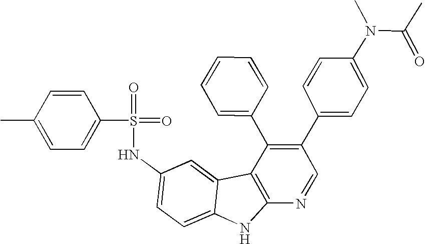 | 3.87 | 561 |
| 40 | 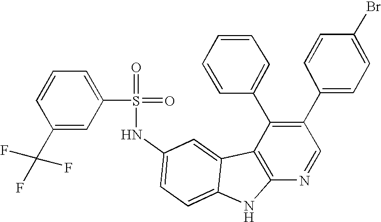 | | 673 |
| 41 | 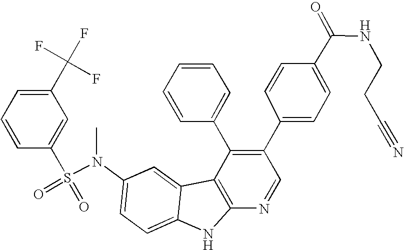 | | 654 |
| 42 | 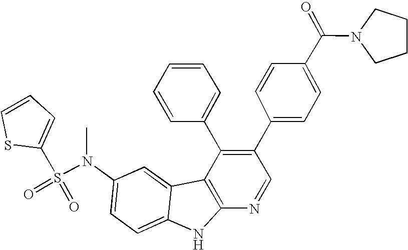 | 4.09 | 593 |
| 43 | 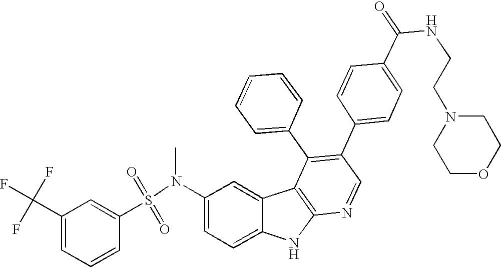 | | 714 |
| 44 | 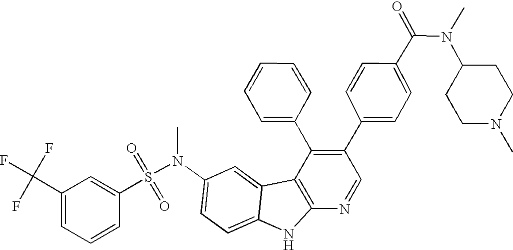 | | 712 |
| 45 | 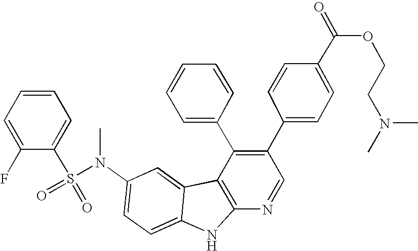 | 3.25 | 623 |
| 46 | 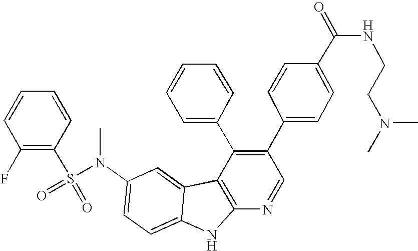 | 3.24 | 622 |
| 47 | 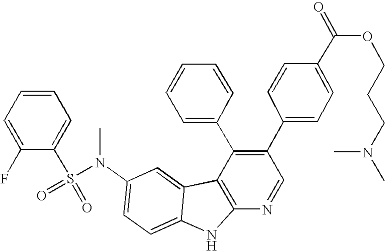 | 3.30 | 637 |
| 48 | 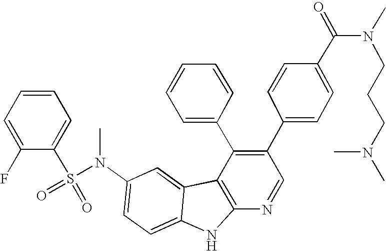 | 3.28 | 650 |
| 49 | 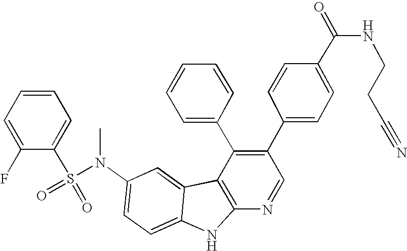 | 3.91 | 604 |
| 50 | 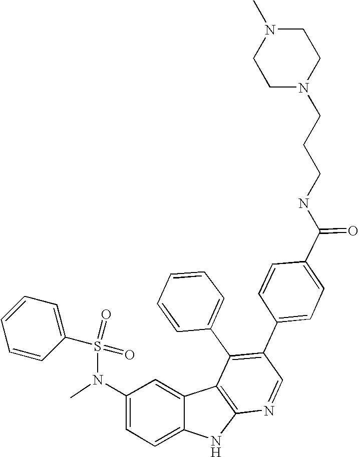 | 2.62 | 673 |
| 51 | 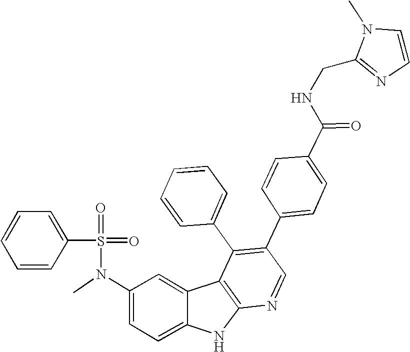 | 2.68 | 627 |
| 52 | 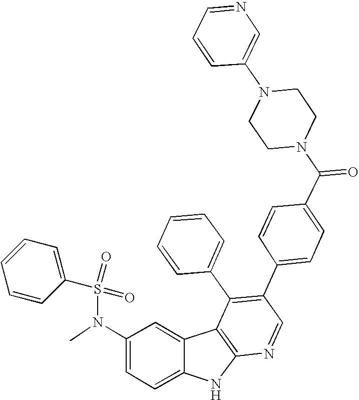 | 2.80 | 679 |
| 53 | 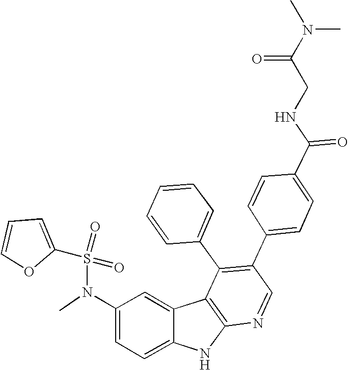 | | 608 |
| 54 | 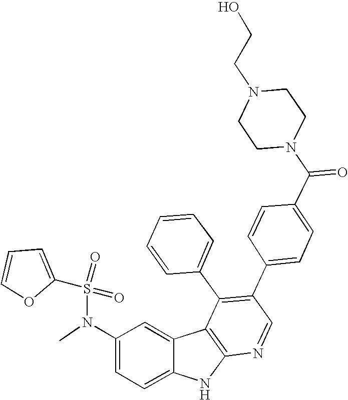 | 2.65 | 636 |
| 55 | 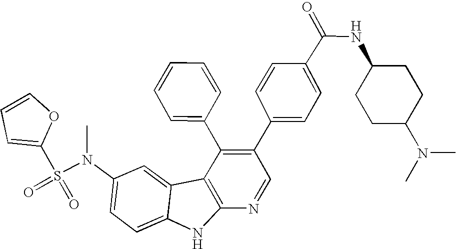 | 2.69 | 648 |
| 56 | 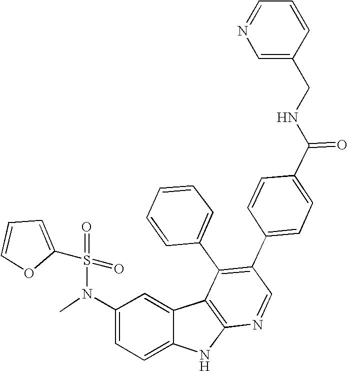 | 2.76 | 614 |
| 57 | 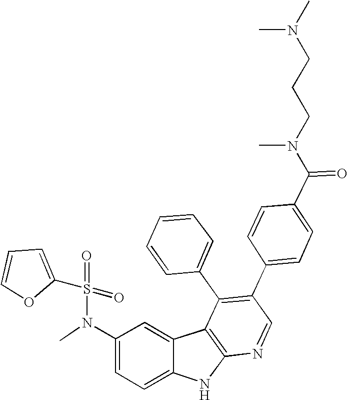 | 2.68 | 622 |
| 58 | 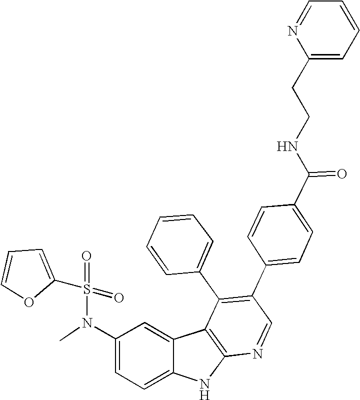 | 2.75 | 628 |
| 59 | 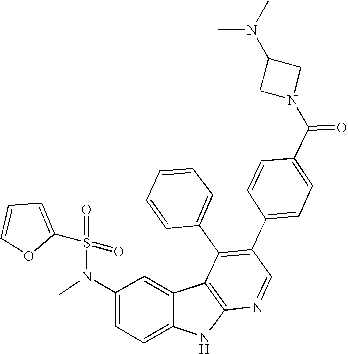 | 2.69 | 606 |
| 60 | 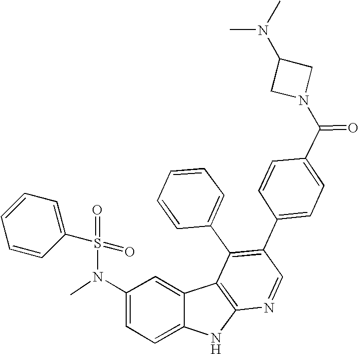 | 2.73 | 616 |
| 61 | 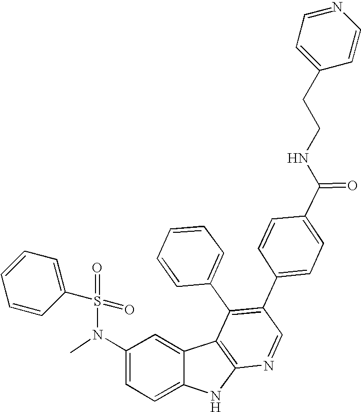 | 2.79 | 638 |
| 62 | 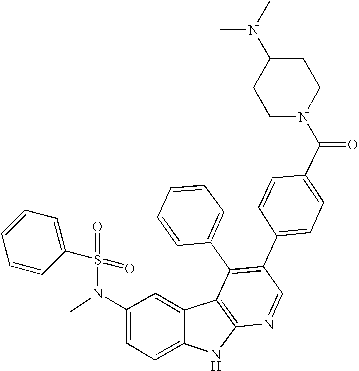 | 2.73 | 644 |
| 63 | 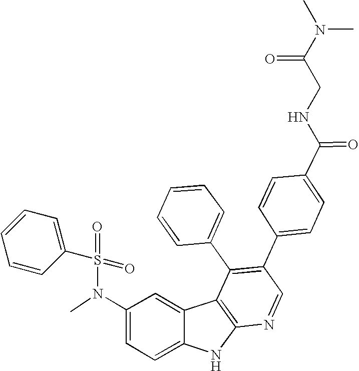 | 3.09 | 618 |
| 64 | 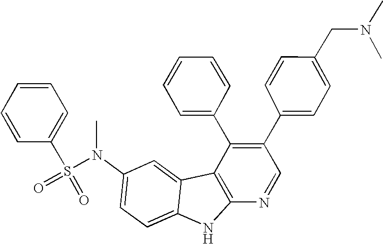 | 2.75 | 547 |
| 65 | 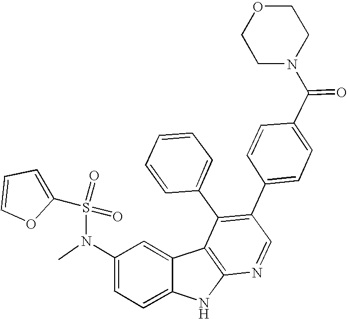 | 3.15 | 593 |
| 66 | 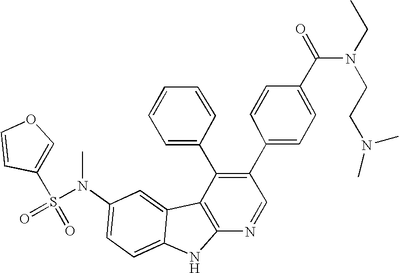 | 2.66 | 622 |
| 67 | 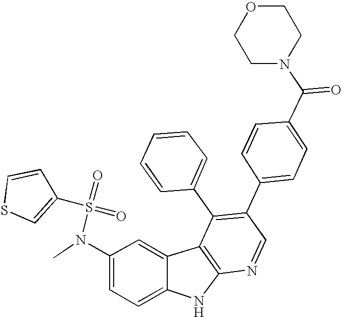 | 3.16 | 609 |
| 68 | 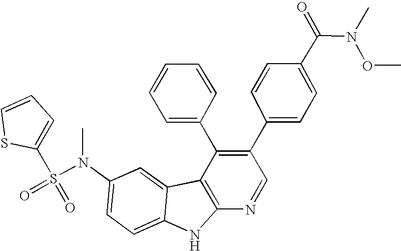 | 3.34 | 583 |
| 69 | 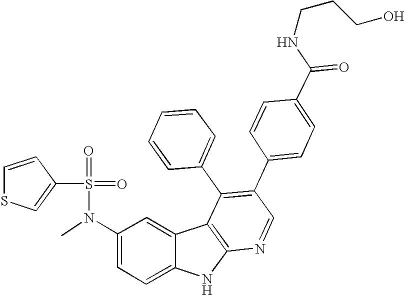 | 2.57 | 597 |
| 70 | 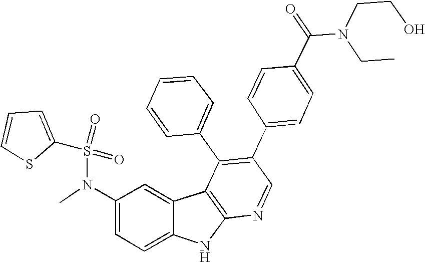 | 3.12 | 611 |
| 71 | 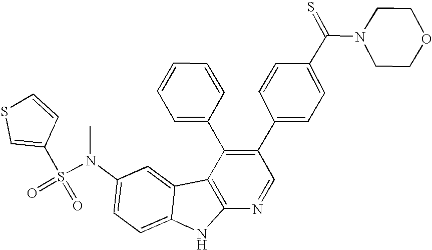 | 3.42 | 625 |
| 72 | 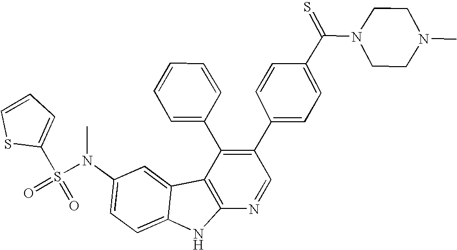 | 2.94 | 638 |
| 73 | 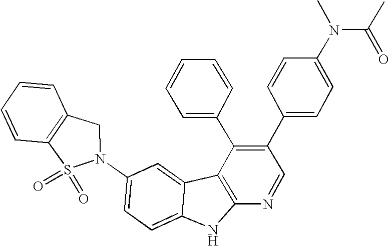 | 2.84 | 575 |
| 74 | 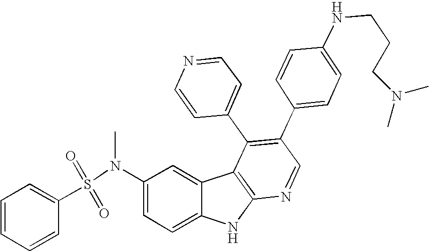 | 2.14 | 591 |
| 75 | 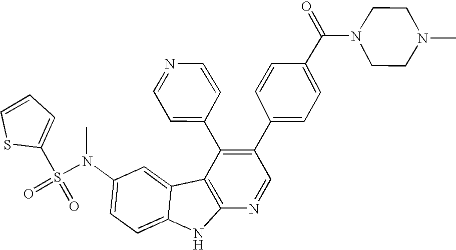 | 2.10 | 623 |
| 76 | 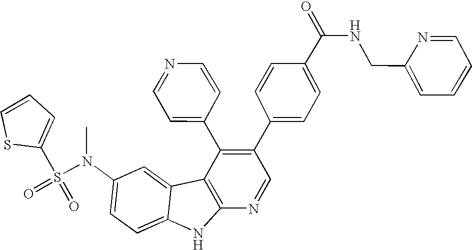 | 2.23 | 631 |
| 78 | 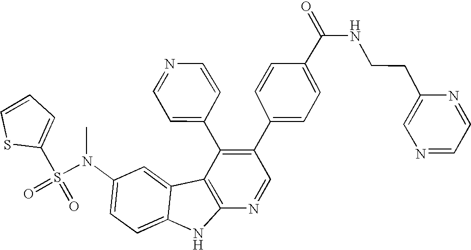 | 2.72 | 646 |
| 79 | 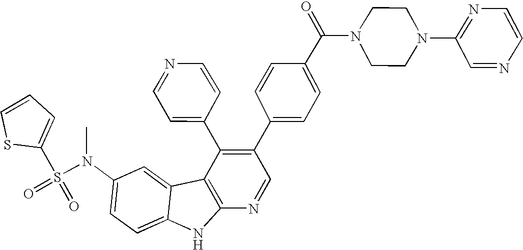 | 2.80 | 687 |
| 80 | 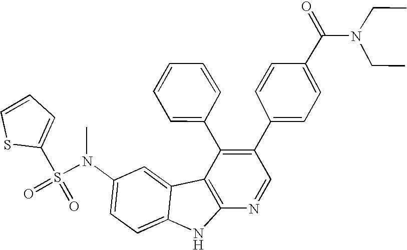 | 3.40 | 595 |
| 81 | 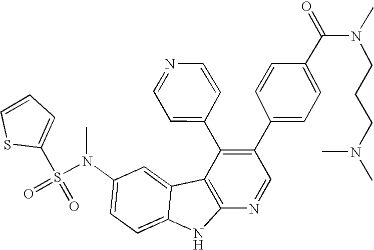 | 2.48 | 639 |
| 82 | 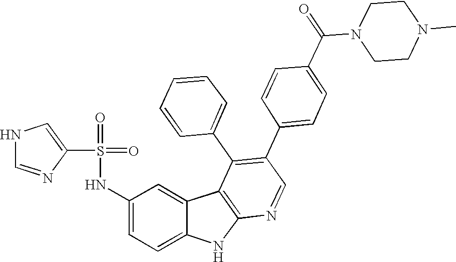 | 2.60 | 592 |
| 83 | 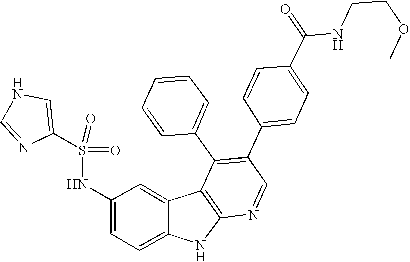 | 2.76 | 567 |
| 84 | 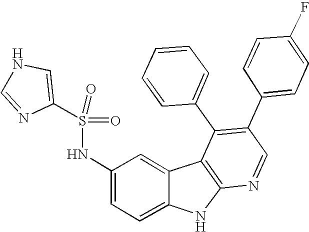 | 3.06 | 484 |
| 85 | 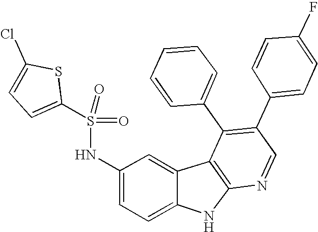 | 3.54 | 535 |
| 86 | 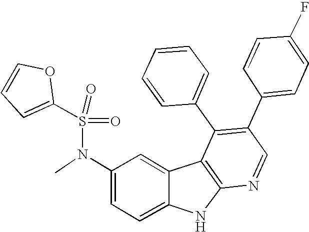 | 3.49 | 498 |
| 87 | 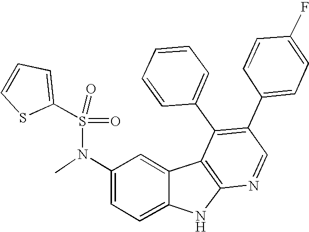 | 3.54 | 514 |
| 88 | 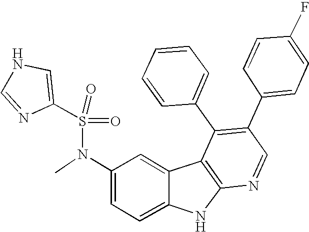 | 3.16 | 498 |
| 89 | 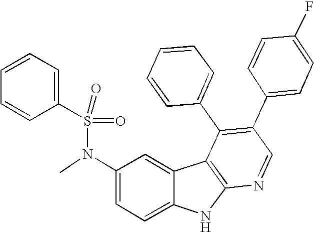 | 3.59 | 508 |
| 90 | 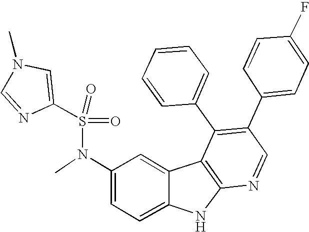 | 3.23 | 512 |
| 91 | 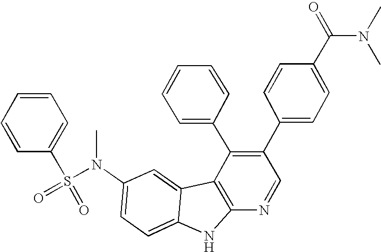 | 3.30 | 561 |
| 92 | 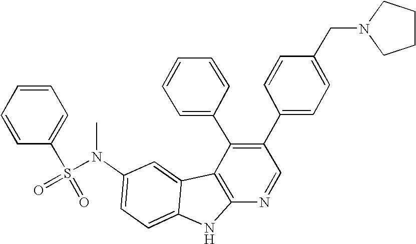 | 2.84 | 573 |
| 93 | 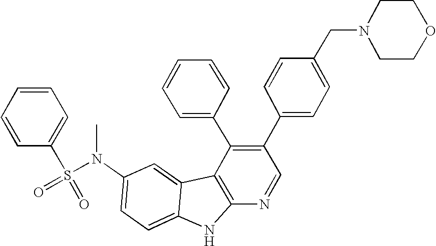 | 2.89 | 589 |
| 94 | 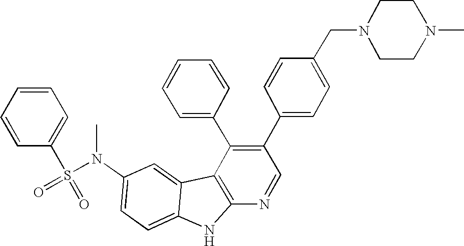 | 2.86 | 602 |
| 95 | 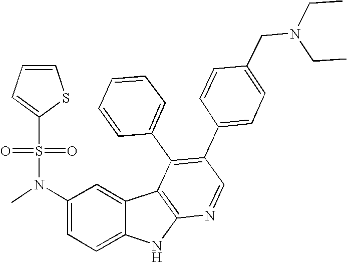 | 2.76 | 581 |
| 96 | 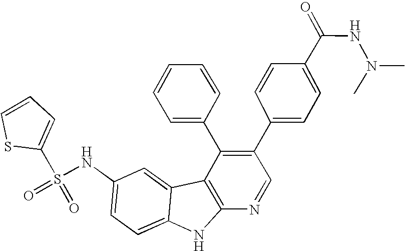 | 2.96 | 568 |
| 97 | 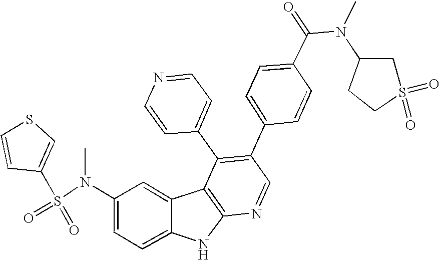 | 2.74 | 672 |
| 98 | 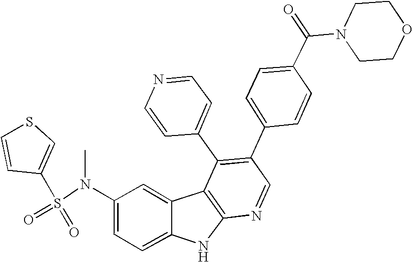 | 2.76 | 610 |
| 99 | 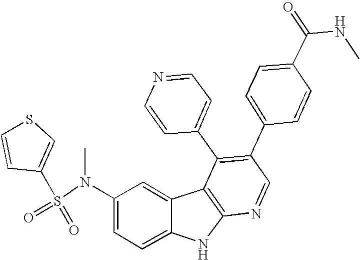 | 2.70 | 554 |
| 100 | 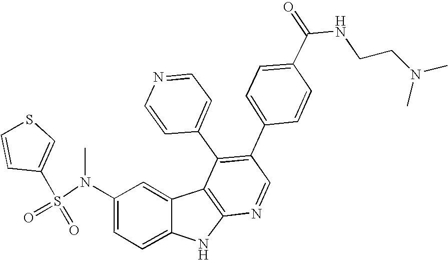 | 2.45 | 611 |
| 101 | 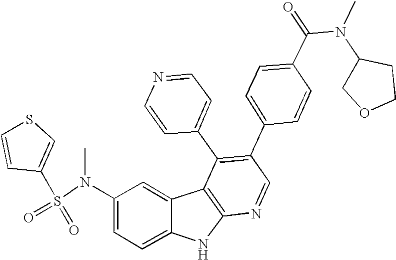 | 2.76 | 624 |
| 102 | 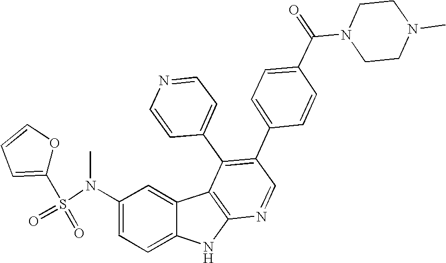 | 2.42 | 607 |
| 103 | 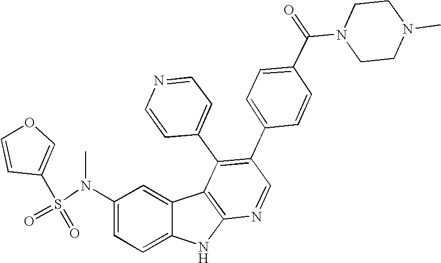 | 2.39 | 607 |
| 104 | 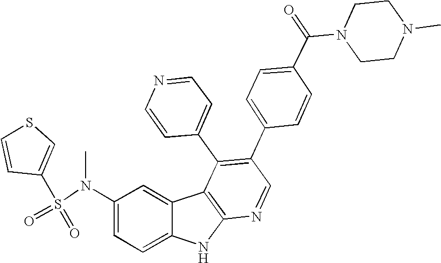 | 2.44 | 623 |
| 105 | 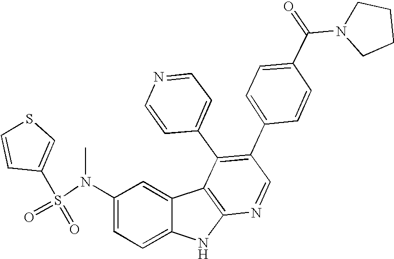 | 2.83 | 594 |
| 106 | 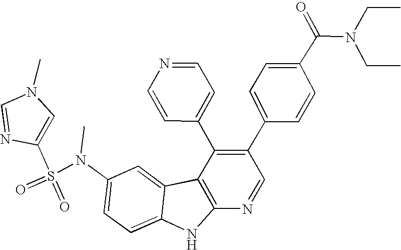 | 3.11 | 593 |
| 107 | 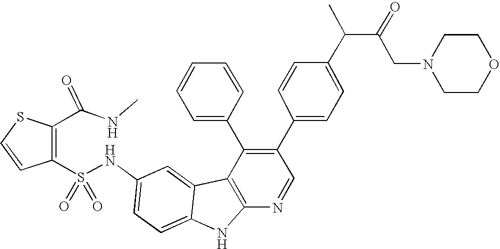 | 2.70 | 695 |
| 108 | 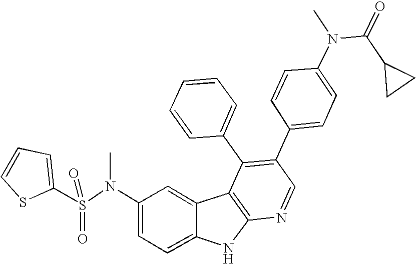 | 3.39 | 593 |
| 109 | 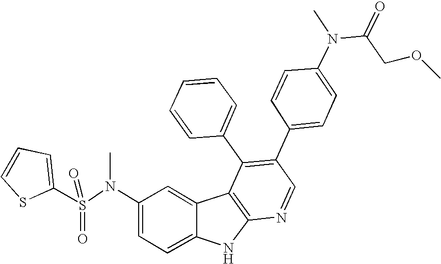 | 3.22 | 597 |
| 110 | 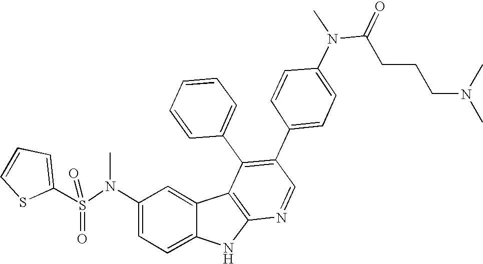 | 2.87 | 638 |
| 111 | 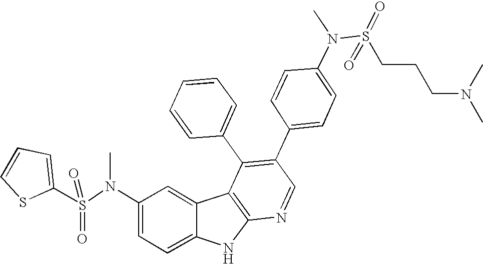 | 2.90 | 674 |
| 112 | 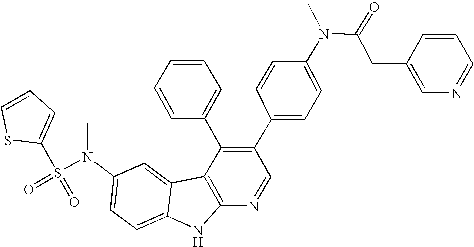 | 2.99 | 644 |
| 113 | 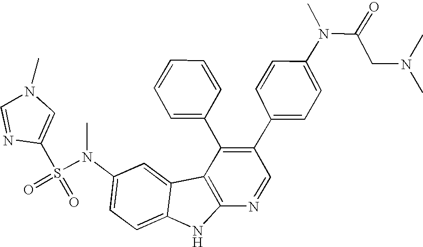 | 2.67 | 608 |
| 114 | 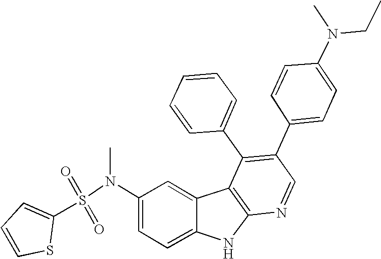 | 3.33 | 553 |
| 115 | 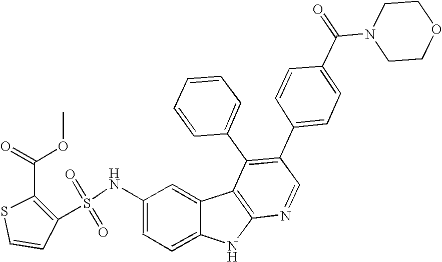 | 3.14 | 653 |
| 116 | 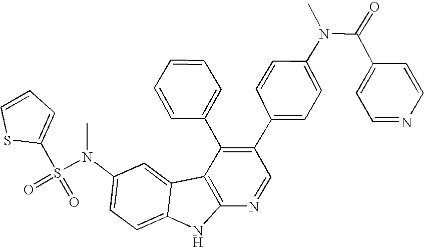 | 3.17 | 630 |
| 117 | 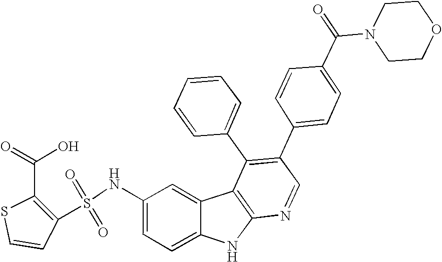 | 3.05 | 639 |
| 118 | 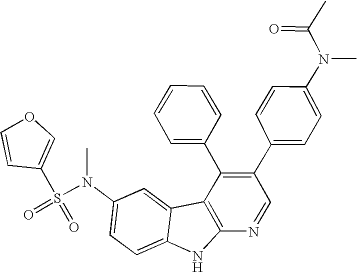 | 3.21 | 551 |
| 119 | 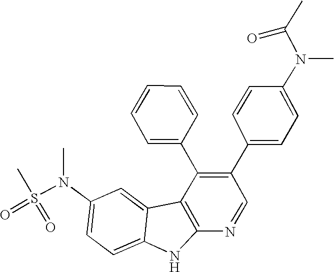 | 3.08 | 499 |
| 120 | 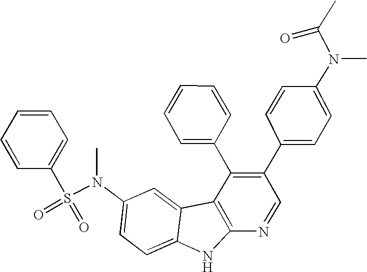 | 3.28 | 561 |
| 121 | 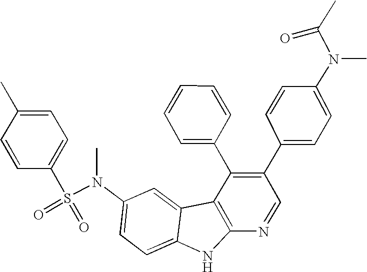 | 3.31 | 575 |
| 122 | 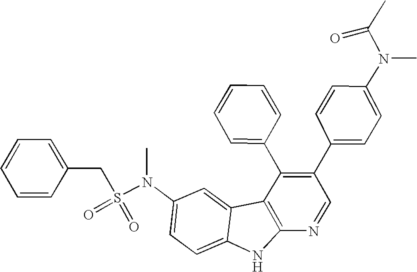 | 3.26 | 575 |
| 123 | 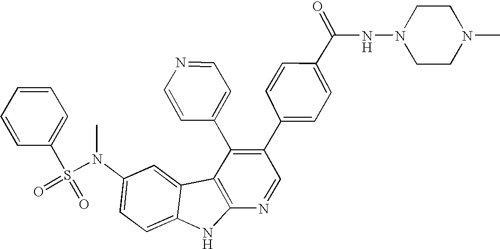 | 2.51 | 632 |
| 124 | 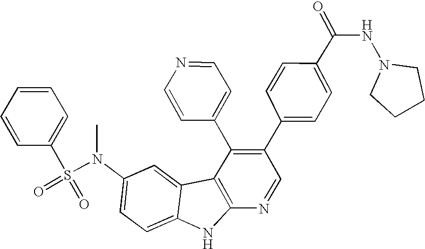 | 2.70 | 603 |
| 125 | 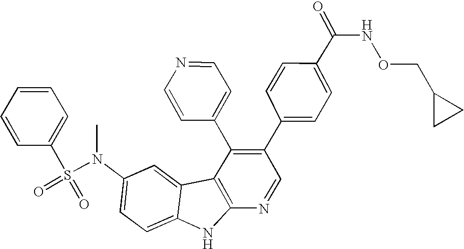 | 2.92 | 604 |
| 126 | 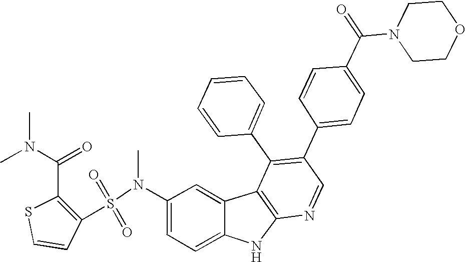 | 3.06 | 680 |
| 127 | 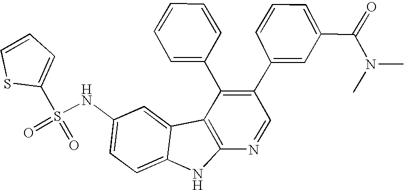 | 3.02 | 553 |
| 128 | 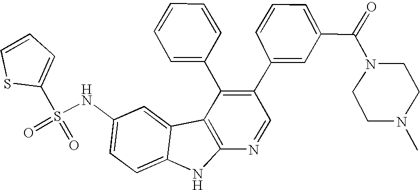 | 2.60 | 608 |
| 129 | 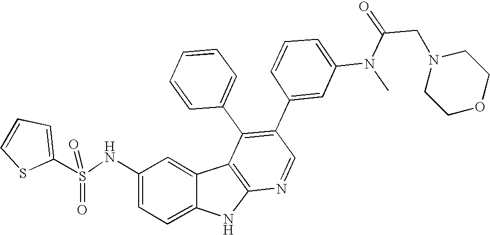 | 2.73 | 638 |
| 130 | 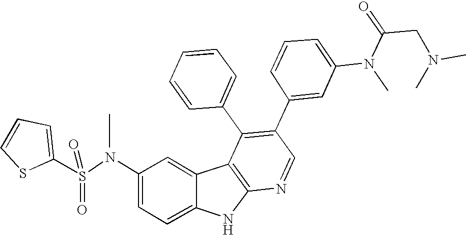 | 2.79 | 610 |
| 131 | 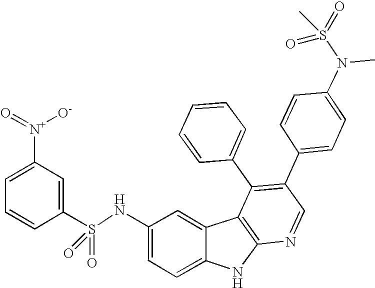 | | 628 |
| 132 | 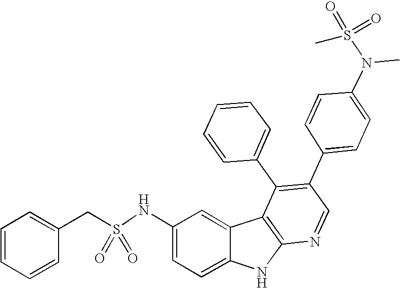 | | 597 |
| 133 | 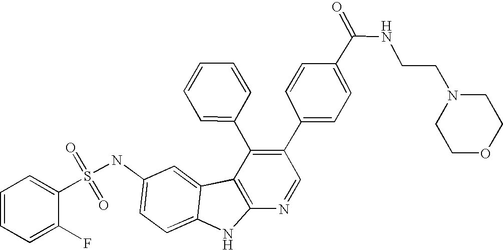 | 3.26 | 664 |
| 134 | 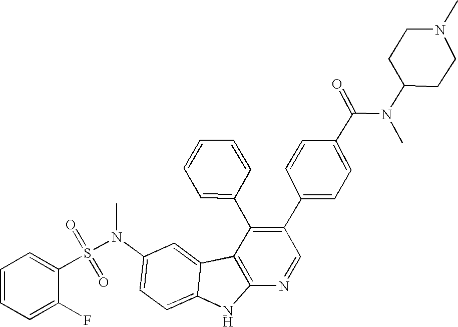 | 3.20 | 662 |
| 135 | 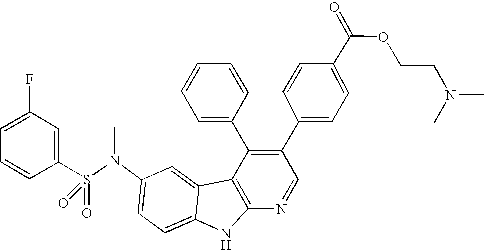 | | 623 |
| 136 | 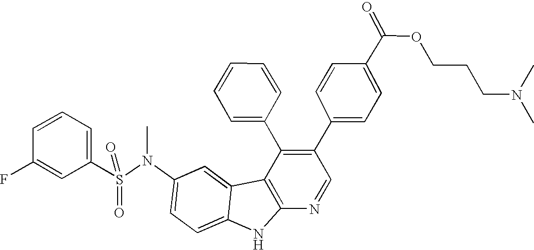 | | 637 |
| 137 | 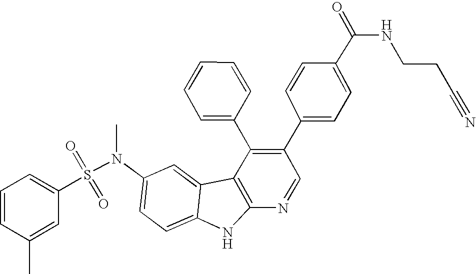 | | 604 |
| 138 | 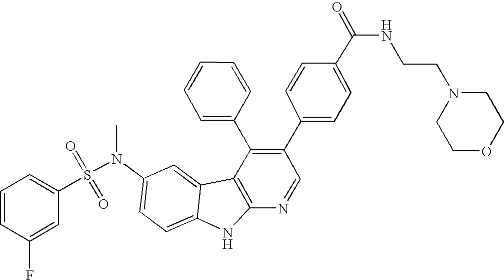 | 3.35 | 664 |
| 139 | 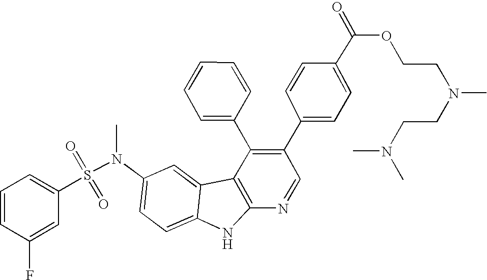 | 3.32 | 680 |
| 140 | 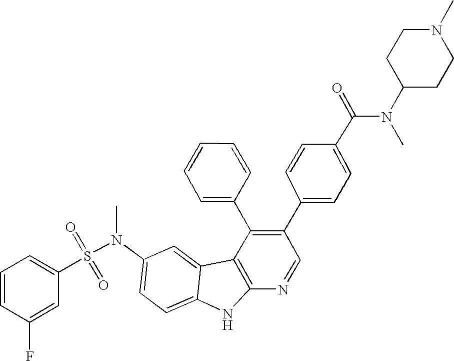 | 3.34 | 662 |
| 141 | 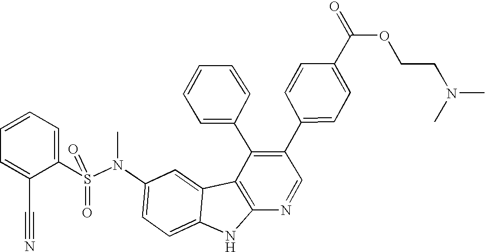 | | 630 |
| 142 | 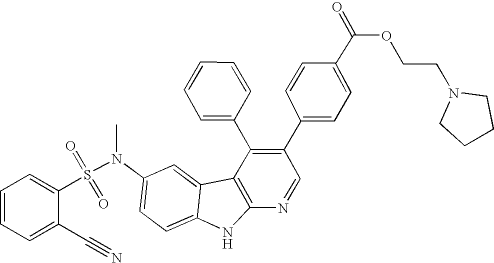 | 3.28 | 656 |
| 143 | 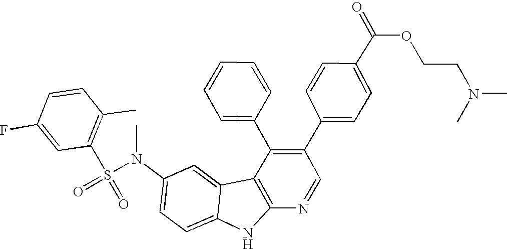 | 3.49 | 637 |
| 144 | 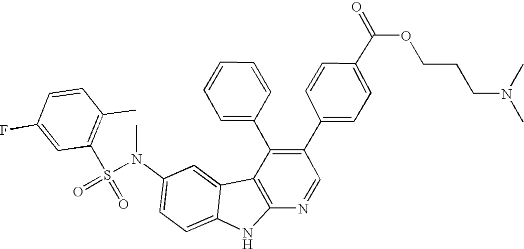 | 3.51 | 651 |
| 145 | 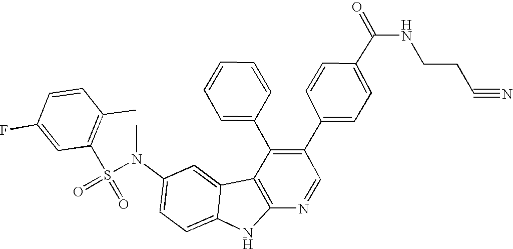 | 4.18 | 618 |
| 146 | 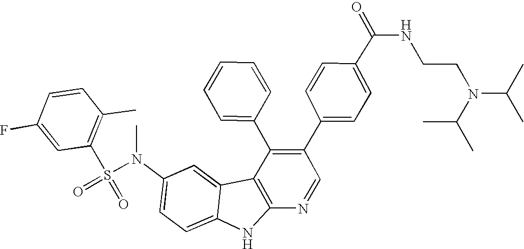 | 3.54 | 692 |
| 147 | 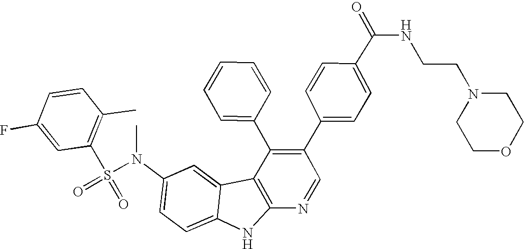 | 3.39 | 678 |
| 148 | 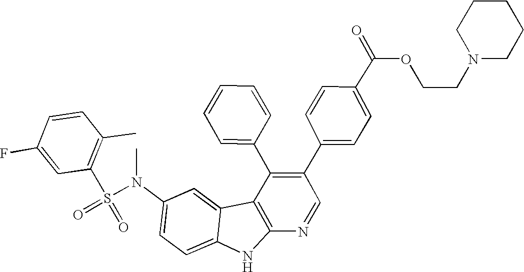 | 3.55 | 677 |
| 149 | 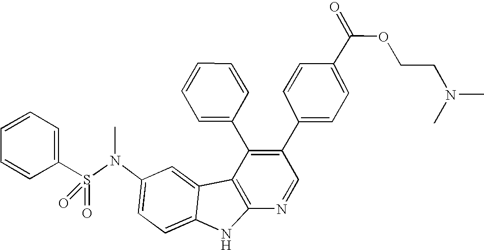 | | 605 |
| 150 | 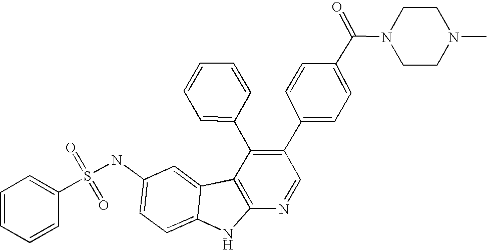 | | 616 |
| 151 | 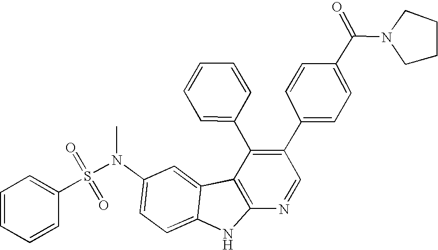 | | 587 |
| 152 | 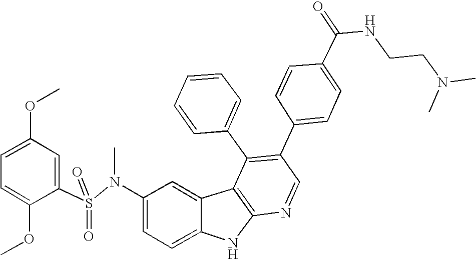 | 3.14 | 664 |
| 153 | 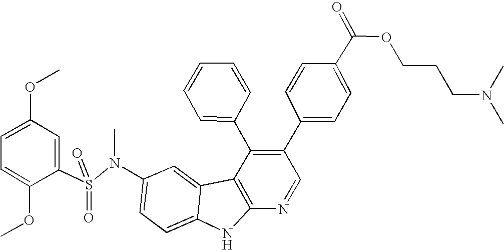 | 3.31 | 679 |
| 154 | 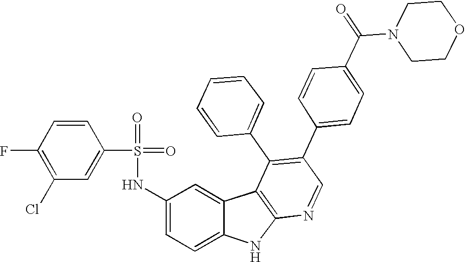 | | 642 |
| 155 | 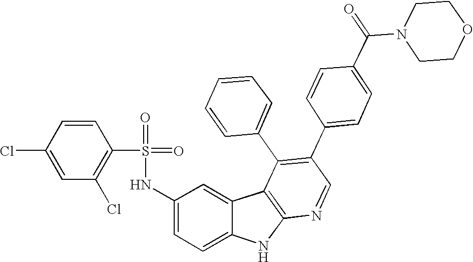 | | 658 |
| 156 | 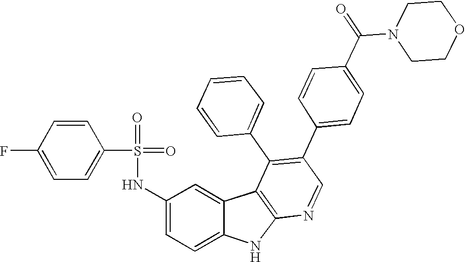 | | 607 |
| 157 | 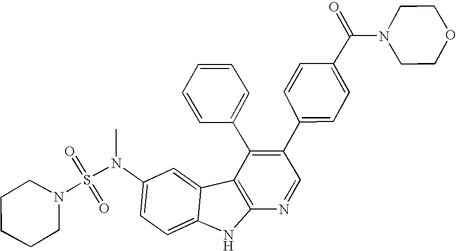 | 3.24 | |
| 158 | 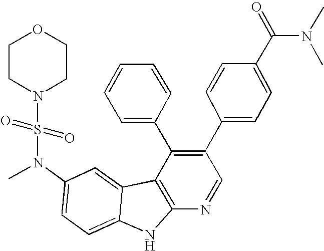 | 3.12 | |
| 159 | 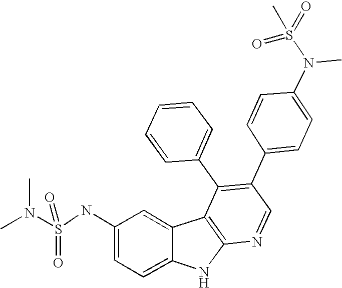 | 3.73 | |
| 160 | 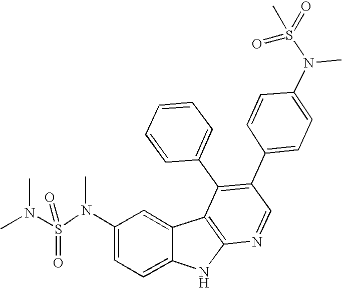 | 4.01 | |
| 161 | 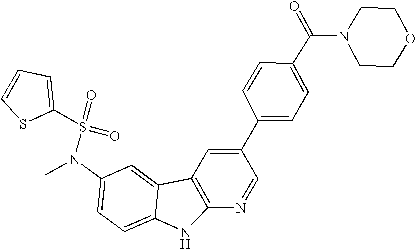 | 3.09 | |
| 162 | 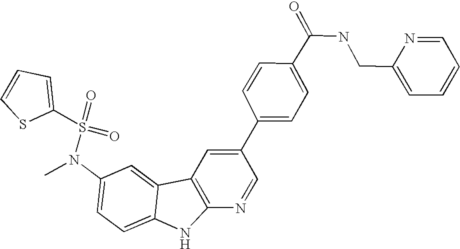 | 2.91 | |
| 163 | 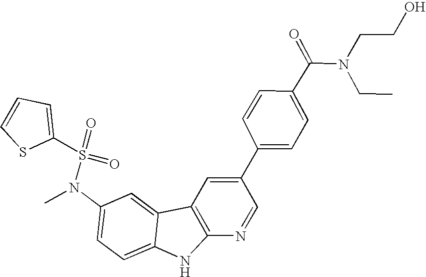 | 3.15 | |
| 164 | 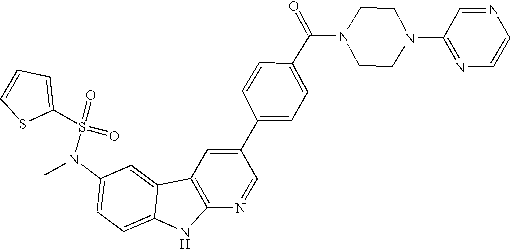 | 3.14 | |
| 165 | 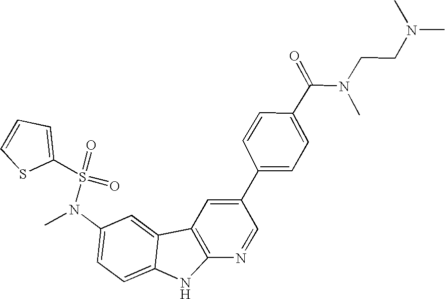 | 2.75 | |
| 166 | 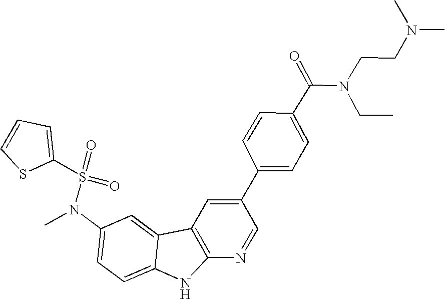 | 2.63 | |
| 167 | 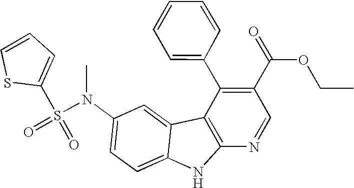 | 2.97 | [M − 1] 490 |
| 168 | 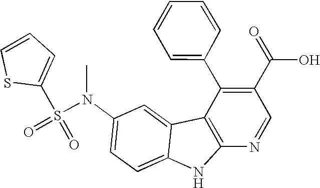 | 2.59 | 464 |
| 169 | 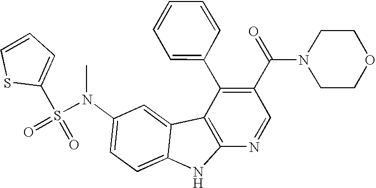 | 2.56 | 533 |
| 170 | 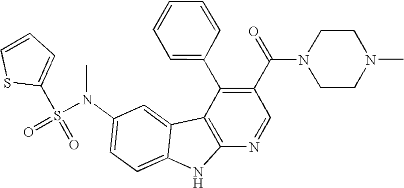 | 2.15 | 546 |
| 171 | 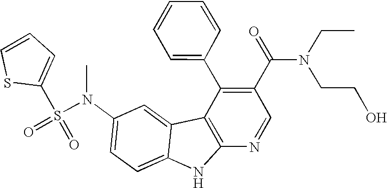 | 2.50 | 535 |
| 172 | 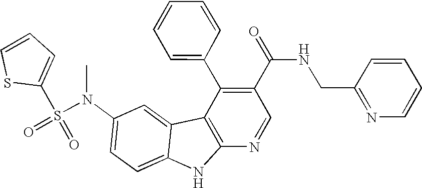 | 2.35 | 554 |
| 173 | 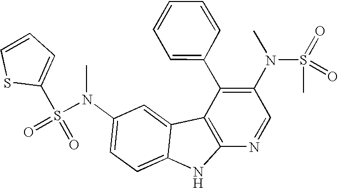 | 3.90 | |

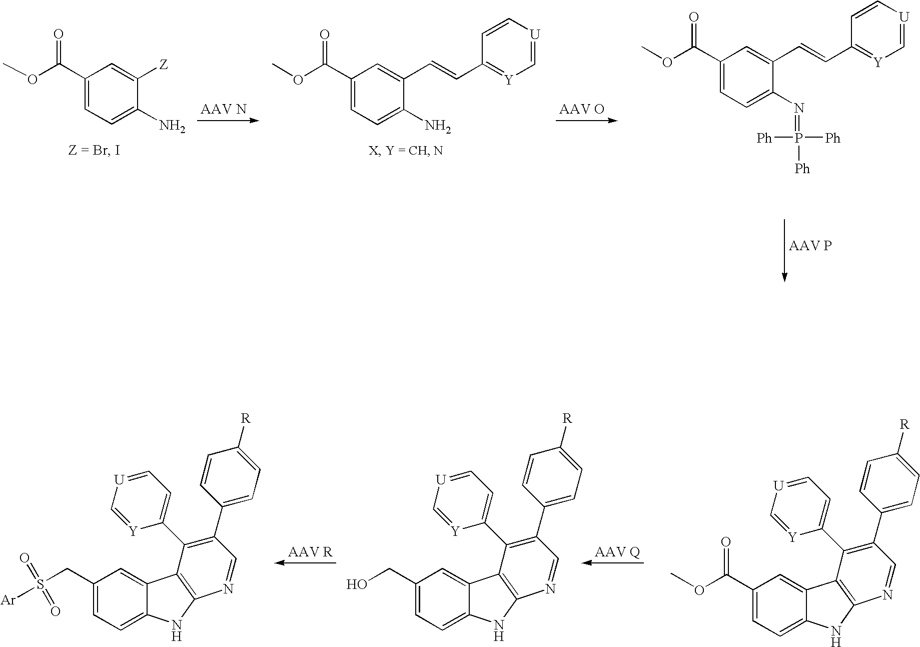

### Preparation of methyl 4-amino-3-(arylethenyl)-benzenecarboxylates (GWM N)

Methyl 4-amino-3-bromobenzenecarboxylate (Costa et al., Heterocycles 1991, 32, 2343-2355) or methyl 4-amino-3-iodobenzenecarboxylate (Spivey et al., J. Org. Chem. 2003, 68, 5, 1843-1851.) (1.1-2 equivalents), Pd(OAc)₂ (0.01-0.05 equivalents) and tri-o-tolylphosphine (0.03-0.05 equivalents) are stirred for 5-12 h at reflux temperature in the presence of a base (triethylamine, cyclohexylmethylamine or N-ethyldiisopropylamine; 1.8 equivalents) under argon in anhydrous DMF, toluene or acetonitrile (2.5-5 mL/1 g 2-bromo-4-nitrobenzenamine). In the event that the reaction stagnates more Pd(OAc)₂ and tri-o-tolylphosphine may be added. The reaction mixture is freed from the solvent using the rotary evaporator, the residue is taken up in EtOAc, filtered through Celite, washed with 1 N NaOH and saturated saline solution, dried (Na₂SO₄), filtered and freed from the solvent using the rotary evaporator. The residue is crystallised from toluene, as a result of which the product is obtained as a solid.

The following intermediate compounds are prepared according to GWM N.

| # | structure | educt |
|---|---|---|
| XVII.1 | 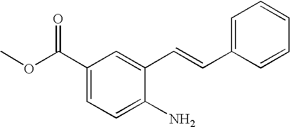 | styrene |
| XVII.2 | 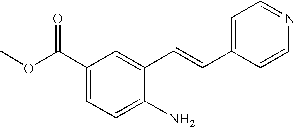 | 4-ethenyl- pyridine |
| XVII.3 | 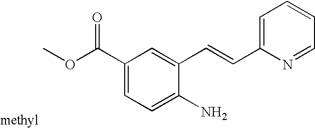 | 2-ethenyl- pyridine |

### Preparation of 2-(2-arylethenyl)-4-triphenyl-phosphoranylideneaminobenzene-carboxylates (GWM O)

Method 1

Diisopropyl or diethyl azodicarboxylate (1.1 equivalents) is added dropwise under argon at 0° C. to a solution of triphenylphosphine (1.1 equivalents) in anhydrous THF (5-15 mL/g amine) and stirred for 1 h. The amine component in anhydrous THF (1-3 mL/g amine) is added and the mixture is stirred for 2-5 h at RT. The reaction mixture is freed from the solvent using the rotary evaporator and fractionally crystallised from EtOAc or purified by chromatography.

Method 2

The amine component is added to a mixture of triphenylphosphine dibromide (1 equivalent) and triethylamine (2 equivalents) in anhydrous toluene (15-25 mL/g amine) under argon and the mixture is stirred for 16-36 h at RT. If the reaction stagnates triphenylphosphine dibromide and triethylamine may be metered in. The solution is diluted with EtOAc (5 mL/100 mL toluene) and stirred with basic aluminium oxide. The mixture is filtered through basic aluminium oxide and the solvent is eliminated using the rotary evaporator. The oily crude product is washed several times with cyclohexane at 55° C. and finally crystallised under cyclohexane.

The following intermediate compounds are prepared according to GWM O.

| # | structure | educt |
|---|---|---|
| XVIII.1 | 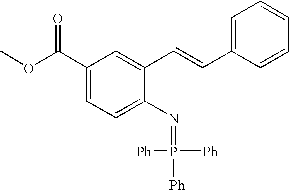 | XVII.1 |
| XVIII.2 | 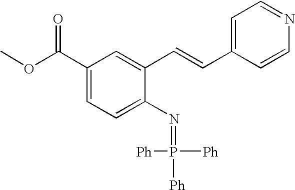 | XVII.2 |
| XVIII.3 | 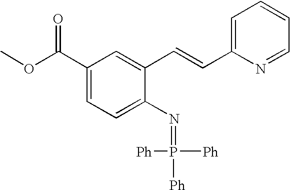 | XVII.3 |

### Cyclisation to form 3,4-biaryl-α-carboline derivatives (GWM P)

Method 1

Phosphoric acid diphenylester azide (1 equivalent) is added dropwise under argon to a mixture of cinnamic acid derivative and triethylamine (1 equivalent) in anhydrous toluene (10-50 mL/g cinnamic acid derivative) and stirred for 12 h at RT. Then the mixture is heated to boiling temperature and stirred for 3 h. The iminophosphorane (0.8 equivalents) is added thereto in solid form, the mixture is stirred for another 4 h and then at this temperature air is piped through the reaction mixture for 12 hours. The reaction mixture is freed from the solvent using the rotary evaporator, taken up in CH₂Cl₂, washed with saturated ammonium chloride solution and saturated saline solution, dried (Na₂SO₄), filtered through silica gel and highly concentrated by evaporation using the rotary evaporator. The residue is fractionally crystallised from EtOAc at −4° C. or purified by chromatography.

Method 2

At 5° C. a mixture of sodium azide (1 equivalent) and tetrabutylammonium chloride (0.1 equivalents) in water (15-25 mL/g sodium azide) is added dropwise to a solution of the substituted cinnamic acid chloride in anhydrous toluene (15-30 mL/1 g cinnamic acid chloride) and the mixture is stirred for 40-90 min at 15-40° C. The organic phase is separated off, dried (Na₂SO₄), filtered and stirred at 100° C. until no more gas is given off. The iminophosphorane (0.8 equivalents) is added in solid form, the mixture is stirred for 4 h and then at this temperature air is piped through the reaction mixture for 12 hours. The reaction mixture is freed from the solvent using the rotary evaporator, taken up in CH₂Cl₂, washed with saturated ammonium chloride solution and saturated saline solution, dried (Na₂SO₄), filtered through silica gel and highly concentrated by evaporation using the rotary evaporator. The residue is fractionally crystallised from EtOAc at −4° C. or purified by chromatography.

The following intermediate compounds are prepared according to GWM P.

| # | structure | cinnamic acid derivative | educt | method |
|---|---|---|---|---|
| XIX.1 | 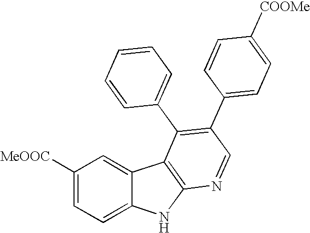 | 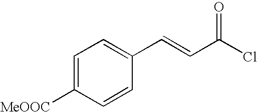 | XVIII.1 | 2 |
| | | WO0187882 | | |
| XIX.2 | 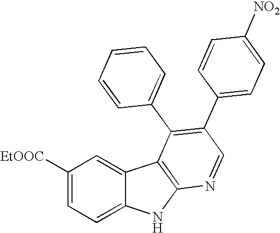 | 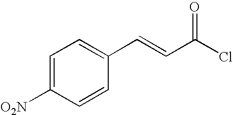 | analogously to XVIII.1 | 1 |
| | | Walpole et al., J. Med. Chem. | | |
| | | 1993, 36(16), 2381-2389 | | |
| XIX.3 | 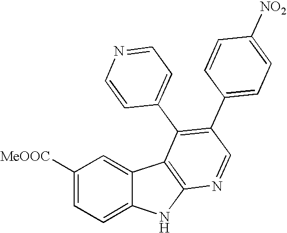 | 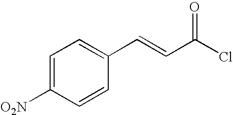 | XVIII.2 | 2 |
| | | Walpole et al., J. Med. Chem. | | |
| | | 1993, 36(16), 2381-2389 | | |
| XIX.4 | 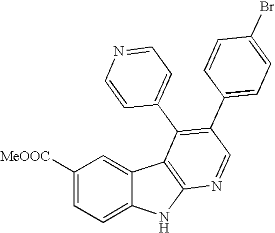 | 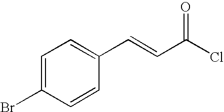 | XVIII.2 | 2 |
| | | Pau et al., Farmaco 2000, | | |
| | | 55(6-7), 439-447 | | |
| XIX.5 | 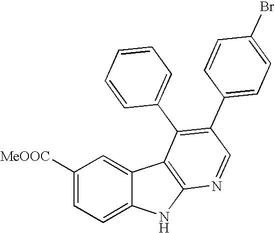 | 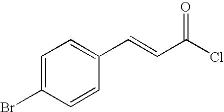 | XVII.1 | 2 |
| | | Pau et al., Farmaco 2000, | | |
| | | 55(6-7), 439-447 | | |
| XIX.6 | 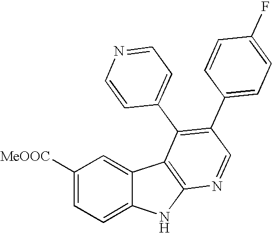 | 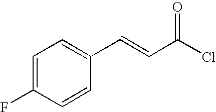 | XVIII.2 | 2 |
| | | Amino et al., Chem. Pharm. | | |
| | | Bull. 1988, 36(11), 4426-4434 | | |
| XIX.7 | 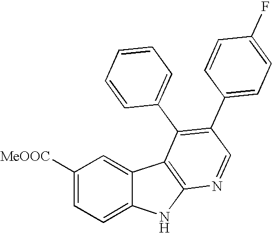 | 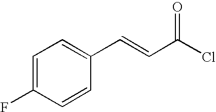 | XVII.1 | 2 |
| | | Amino et al., Chem Pharm. | | |
| | | Bull. 1988, 36(11), 4426-4434 | | |
| XIX.8 | 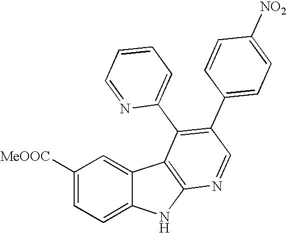 | 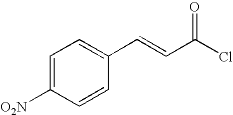 | XVII.3 | 2 |
| | | Walpole et al., J. Med. Chem. | | |
| | | 1993, 36(16), 2381-2389 | | |

### Reduction of Carboline-Carboxylic Acid Esters to the Alcohol (GWM Q)

Diisobutylaluminium hydride (DIBAL-H) (20% in toluene; 3-5 equivalents) is added at 0° C. to a solution of the carboline ester in anhydrous THF (20-40 mL/g educt) and stirred for 3-12 h at RT. If the reaction stagnates reducing agent is metered in. The mixture is hydrolysed with water and 15% NaOH until a precipitate is obtained which is separated off by filtration and decocted with methanol. The combined organic phases are freed from the solvent using the rotary evaporator, taken up in CH₂Cl₂, washed with water and saturated saline solution, dried (Na₂SO₄), filtered, freed from the solvent using the rotary evaporator and purified by chromatography or by crystallisation. Reduction may also be carried out analogously thereto with lithium aluminium hydride.

The following intermediate compounds are prepared according to GWM Q.

| # | structure | educt |
|---|---|---|
| XX.1 | 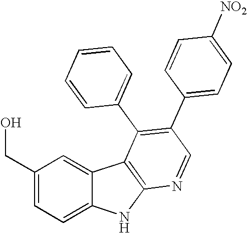 | XIX.2 |
| XX.2 | 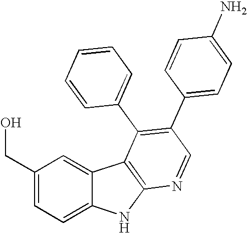 | 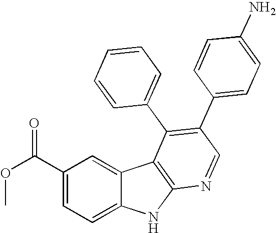 |
| XX.3 | 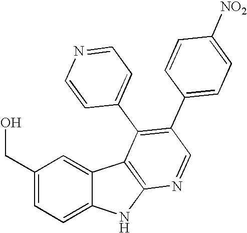 | XIX.3 |
| XX.4 | 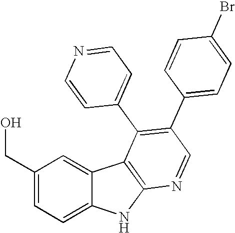 | XIX.4 |
| XX.5 | 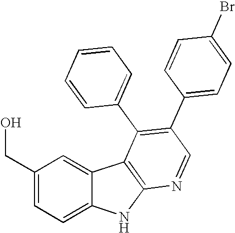 | XIX.5 |
| XX.6 | 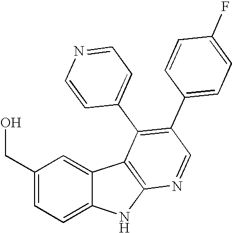 | XIX.7 |

### Reaction of the Alcohol with Sulphinic Acid Salts to the Sulphone (GWM R)

Method 1

Arylsulphinic acid sodium salt (3-10 equivalents) is added in solid form to a suspension of the starting compound in 3-5 N aqueous hydrochloric acid (10-100 mL/g educt) and the mixture is stirred for 2-12 h at 100° C. The product is obtained by extraction or filtration and purified by crystallisation or chromatography.

Method 2

Arylsulphinic acid sodium salt (3-10 equivalents) is added in solid form to a suspension of the starting compound in formic acid (5-20 mL/g educt) and the mixture is stirred for 2-24 h at 100° C. The mixture is evaporated down, poured onto water and neutralised with potassium carbonate. The product is obtained by extraction or filtration and purified by crystallisation or chromatography.

The following intermediate compounds are prepared according to GWM R.

| # | structure | educt |
|---|---|---|
| XXI.1 | 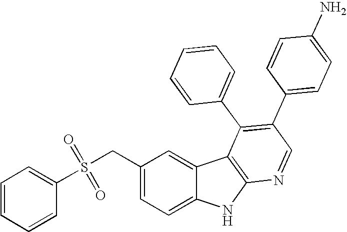 | XX.2 |
| XXI.2 | 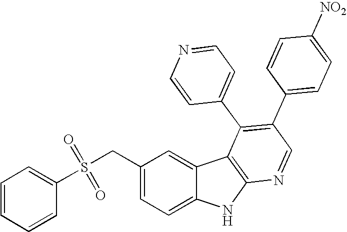 | XX.3 |
| XXI.3 | 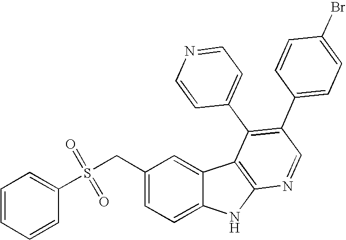 | XX.4 |
| XXI.4 | 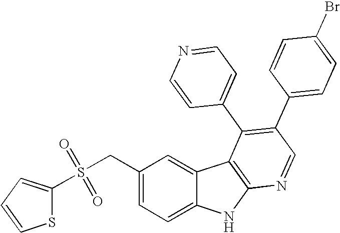 | XX.4 |
| XXI.5 | 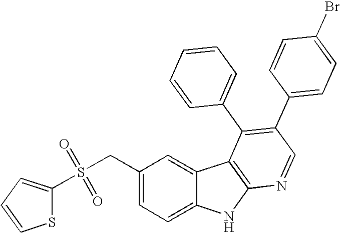 | XX.5 |
| XXI.6 | 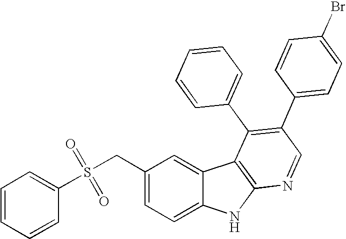 | XX.5 |
| XXI.7 | 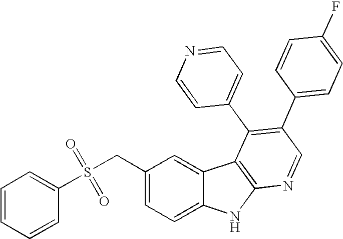 | XX.6 |

### Reduction of Nitrocarboline Derivatives to the Corresponding Amines (GWM S)

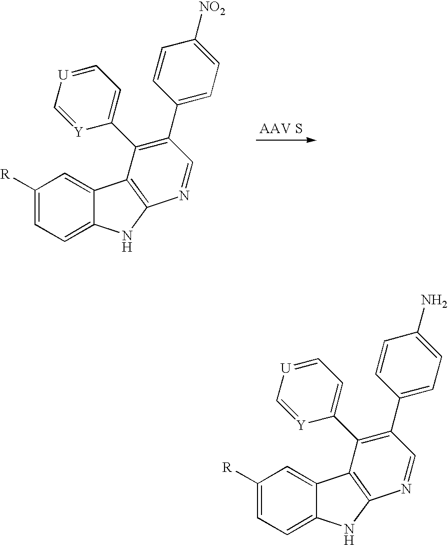

A mixture of nitro compound and palladium on activated charcoal (5% or 10%) or Raney nickel (5-25 mg/g nitro compound) in methanol, THF, 50% methanol in THF or DMF is hydrogenated under a hydrogen pressure of 3 to 10 bar at a temperature between 15 and 60° C. over a period of 3-48 h. The reaction mixture is degassed with nitrogen and the catalyst is filtered off through Celite. The solvent is eliminated using the rotary evaporator and the residue is optionally purified by chromatography.

The following intermediate compounds are prepared according to GWM S.

| # | structure | educt |
|---|---|---|
| XXII.1 | 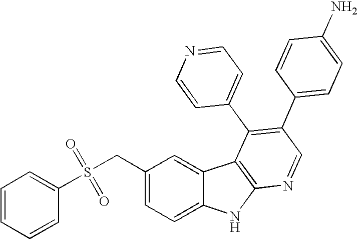 | XXI.2 |
| XXII.2 | 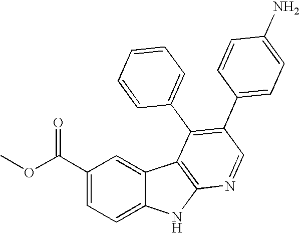 | 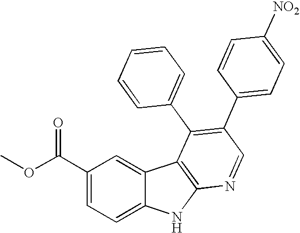 |

### Preparation of 4-nitrophenyl arylsulphonates (GWM T)

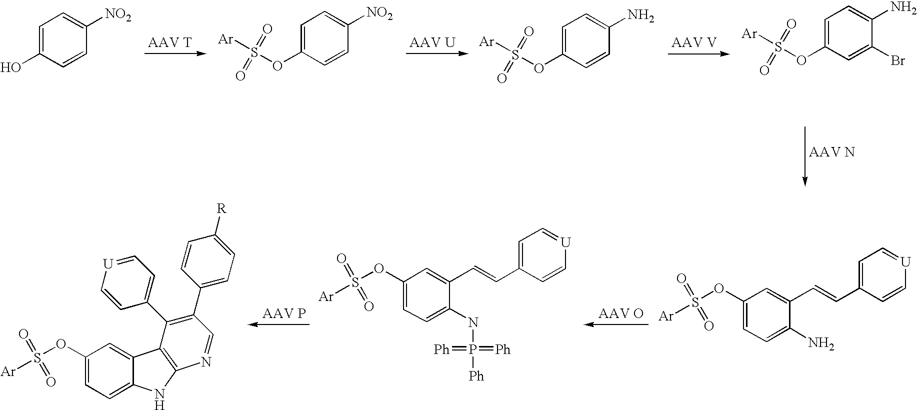

Triethylamine (1-2 equivalents) and 4-nitrophenol in anhydrous CH₂Cl₂ (2-10 mL/g 4-nitrophenol) are added successively at 0° C. to a solution of the sulphonic acid chloride in anhydrous CH₂Cl₂ (0.5-10 mL/g sulphonic acid chloride) and the mixture is stirred for 12-48 h at RT. If the reaction stagnates sulphonic acid chloride and base are metered in.

Working Up Method 1

The precipitate formed is separated off by filtration, the filtrate is highly concentrated by evaporation, any precipitated product is filtered off and optionally purified by chromatography.

Working Up Method 2

The precipitate formed is separated off by filtration, the filtrate is diluted with CH₂Cl₂ and washed with 1 N HCl, water and saturated saline solution, dried (Na₂SO₄), filtered and freed from the solvent using the rotary evaporator. The residue is optionally purified by chromatography.

The following intermediate compounds are prepared according to GWM T.

| # | structure |
|---|---|
| XXIII.1 | 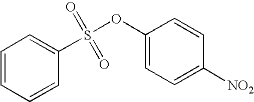 |
| | Choi et al., J. Org. Chem. 2002, |
| | 67, 1277-1281 |
| XXIII.2 | 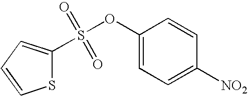 |
| | El-Maghraby et al., J. Chem. Techn. |
| | Biotechn. 1983, 33A(1), 25-32 |
| XXIII.3 | 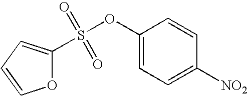 |

### Reduction of Nitrocarboline Derivatives (GWM U)

A mixture of nitro compound and palladium on activated charcoal (5% or 10%) in methanol, THF, 50% methanol in THF or DMF is hydrogenated under a hydrogen pressure of 3 to 10 bar at a temperature between 15-60° C. over a period of 3 to 168 h. The reaction mixture is degassed with nitrogen and the catalyst is filtered off through Celite. The solvent is eliminated using the rotary evaporator and the residue is optionally purified by chromatography.

The following intermediate compounds are prepared according to GWM U.

| # | structure | educt |
|---|---|---|
| XXIV.1 | 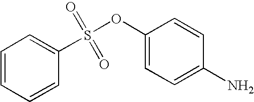 | XXIII.1 |
| | Tappe, H. Synthesis 1980, 7, | |
| | 577-578 | |
| XXIV.2 | 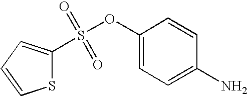 | XXIII.2 |
| XXIV.3 | 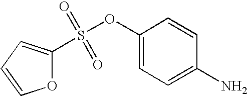 | XXIII.3 |

### Bromination (GWM V)

N-bromosuccinimide (NBS) (1-1.1 equivalents) in anhydrous DMF (5-10 mL/g NBS) is slowly added dropwise at −15 to 0° C. to a solution of the amine in anhydrous DMF (5-20 mL/1 g amine) and stirred for 2-5 h at RT. The reaction mixture is poured onto water, stirred for 1-3 h and the precipitate is obtained by filtration. If no crystals are obtained the product is isolated by extraction and optionally purified by chromatography.

The following intermediate compounds are prepared according to GWM I.

| # | structure | educt |
|---|---|---|
| XXV.1 | 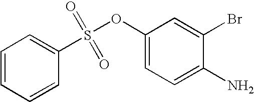 | XXIV.1 |
| XXV.2 | 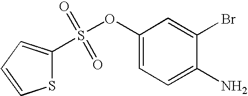 | XXIV.2 |
| XXV.3 | 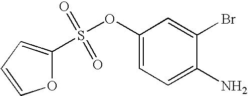 | XXIV.3 |

Aryl-[4-amino-3-(arylethenyl)phenyl]sulphonic acid esters are prepared analogously to GWM N.

| # | structure | educt |
|---|---|---|
| XXVI.1 | 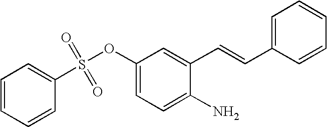 | XXV.1 |
| XXVI.2 | 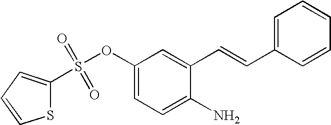 | XXV.2 |
| XXVI.3 | 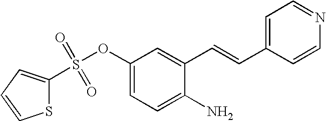 | XXV.2 |
| XXVI.4 | 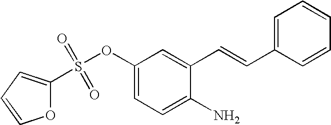 | XXV.3 |

Aryl-[2-(2-arylethenyl]-4-triphenylphosphoranylidene-amino)-phenyl]-phenyl]-sulphonic acid esters are prepared according to GWM O.

| # | structure | Method | educt |
|---|---|---|---|
| XXVII.1 | 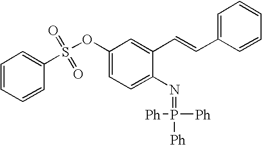 | 2 | XXVI.1 |
| XXVII.2 | 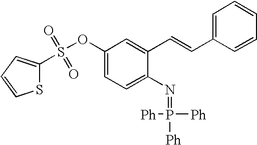 | 2 | XXVI.2 |
| XXVII.3 | 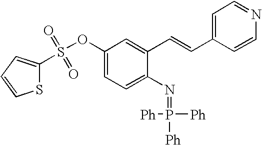 | 1 | XXVI.3 |
| XXVII.4 | 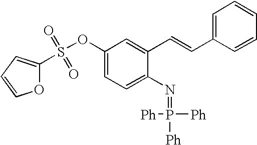 | 1 | XXVI.4 |

The cyclisation to form 3,4-biaryl-α-carboline derivatives is carried out according to GWM P.

The following intermediate compounds are prepared according to GWM P, Method 2.

| # | structure | cinnamic acid derivative | educt |
|---|---|---|---|
| XXVIII.1 | 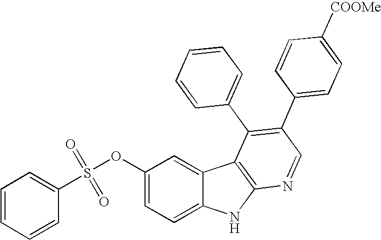 | 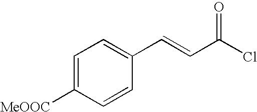 | XXVII.1 |
| | | WO017882, | |
| XXVIII.2 | 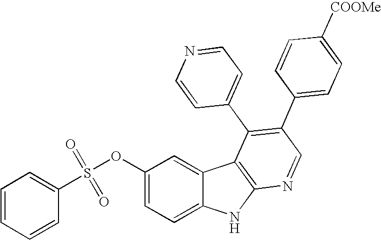 | 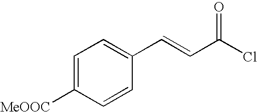 | analogously to XXVII.3 |
| | | WO017882, | |
| XXVIII.3 | 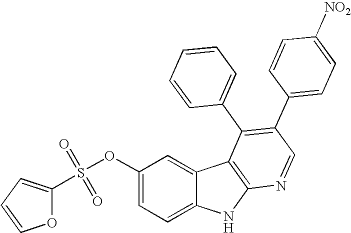 | 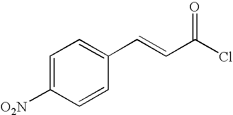 | XXVII.4 |
| | | Walpole et al., J. | |
| | | Chem. 1993, 36(16), | |
| | | 2381-2389 | |
| XXVIII.4 | 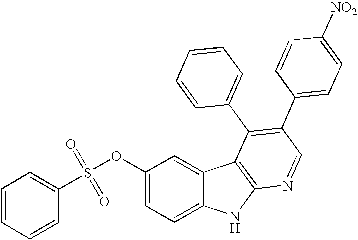 | 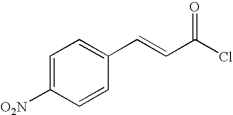 | XXVII.1 |
| | | Walpole et al., J. | |
| | | Chem. 1993, 36(16), | |
| | | 2381-2389 | |
| XXVIII.5 | 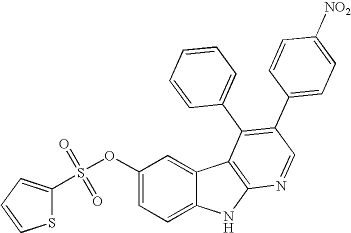 | 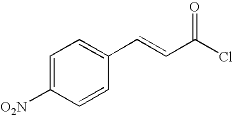 | XXVII.2 |
| | | Walpole et al., J. | |
| | | Chem. 1993, 36(16), | |
| | | 2381-2389 | |
| XXVIII.6 | 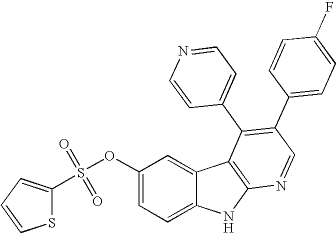 | 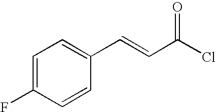 | XXVII.3 |
| | | Amino et al., Chem. | |
| | | Pharm. Bull. 1988, | |
| | | 36(11), 4426-4434 | |
| XXVIII.7 | 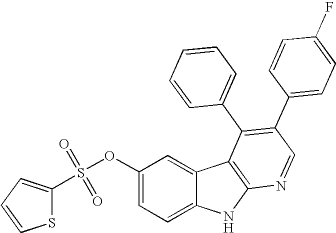 | 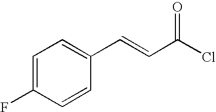 | XXVII.2 |
| | | Amino et al., Chem. | |
| | | Pharm. Bull. 1988, | |
| | | 36(11), 4426-4434 | |

The reduction of the nitrocarboline derivatives to form the amine is carried out according to GWM S.

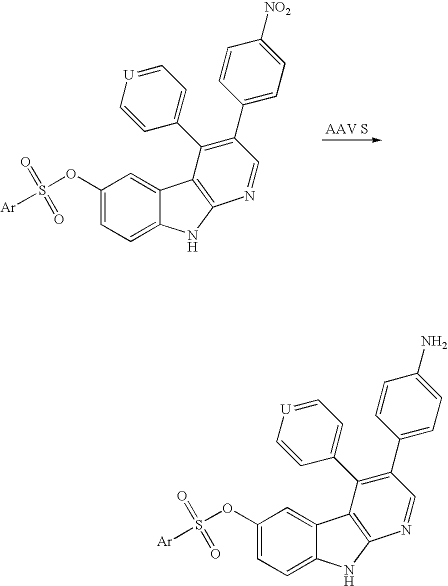

The following intermediate compounds are prepared according to GWM S.

| # | structure | educt |
|---|---|---|
| XXIX.1 | 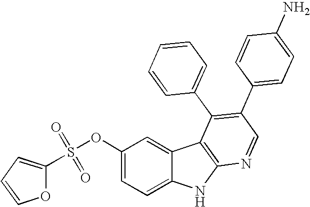 | XXVIII.3 |
| XXIX.2 | 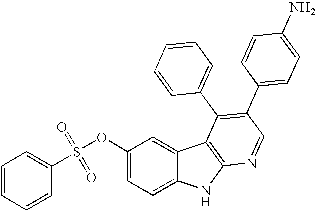 | XXVIII.1 |
| XXIX.3 | 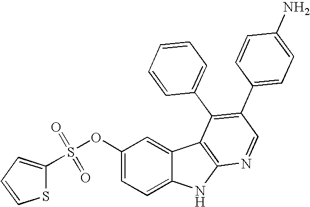 | XXVIII.5 |

### Formylation of Carbolinamines (GWM W1)

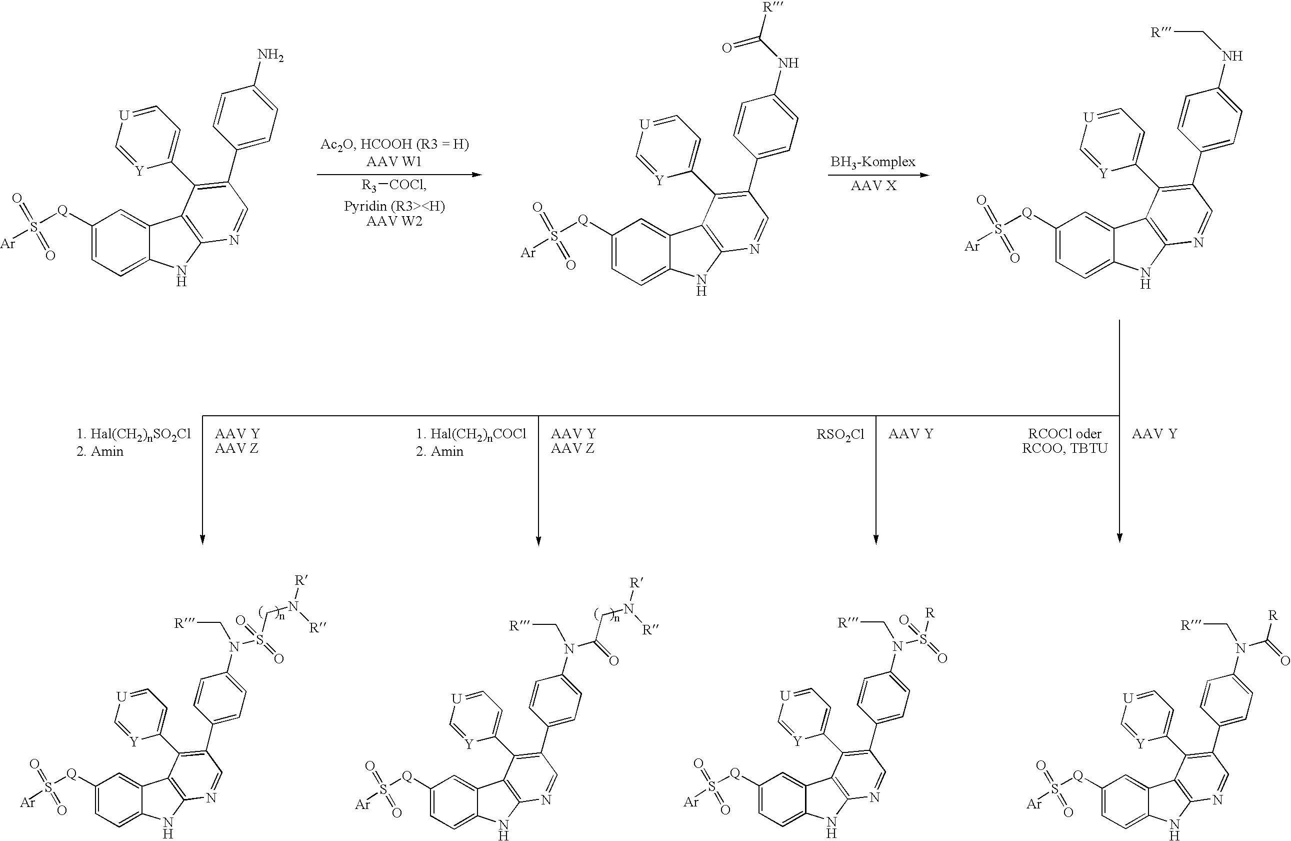

Formic acid (10 mL/g educt) and acetic anhydride (2-5 equivalents) are stirred for 1-5 h at 10-50° C. and diluted with anhydrous THF (20-30 mL/g educt). Then the amine is added batchwise over a period of 10 min and the mixture is stirred for 1 h at RT. The product is obtained either by precipitation with tert-butylmethylether or by extraction and optionally purified by chromatography.

The following intermediate compounds are prepared according to GWM W1.

| # | structure | educt |
|---|---|---|
| XXX.1 | 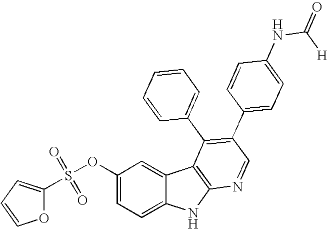 | XXIX.1 |
| XXX.2 | 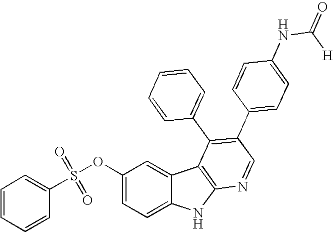 | XXIX.2 |
| XXX.3 | 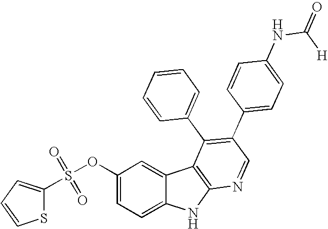 | XXIX.3 |

### Acylation of Carbolinamines (GWM W2)

A solution of XXXVII.1 (100 mg, 0.2 mol) and acid chloride or acid anhydride (0.27 mmol, 1.3 equivalents) in 2 mL pyridine is stirred for 2-5 h at RT. It is mixed with three times the volume of water, the precipitate is suction filtered and washed with 1 N hydrochloric acid and water and dried in vacuo at 60° C.

The following intermediate compounds are prepared according to GWM W2.

| # | structure | educt |
|---|---|---|
| XXXI.1 | 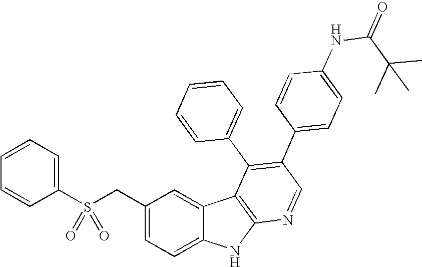 | XXI.1 |
| XXXI.2 | 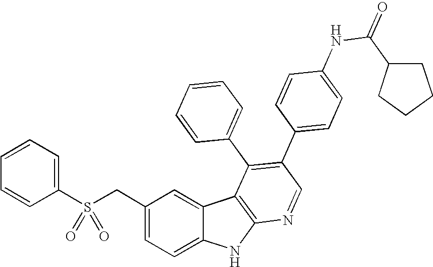 | XXI.1 |
| XXXI.3 | 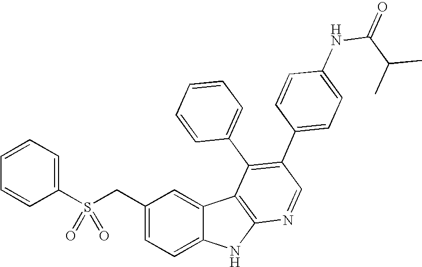 | XXI.1 |
| XXXI.4 | 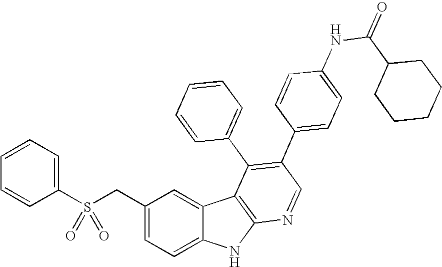 | XXI.1 |
| XXXI.5 | 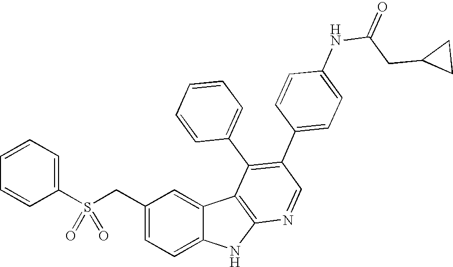 | V.1 |
| XXXI.6 | 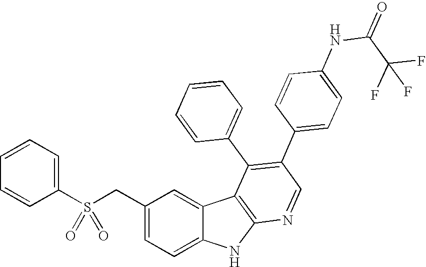 | XXI.1 |
| XXXI.7 | 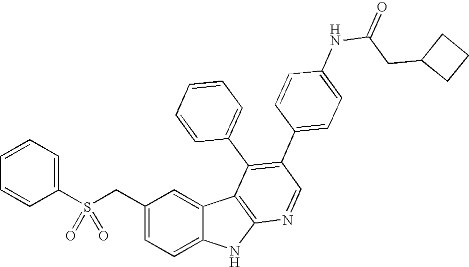 | XXI.1 |

### Reduction to N-methylcarbolinamines (GWM X)

Borane-dimethylsulphide complex or borane-THF complex (2-20 equivalents) is added dropwise at RT to a solution of the starting compound in anhydrous THF (10-50 mL) and the mixture is stirred for 2-10 h at RT. Then additional borane complex is optionally added dropwise and the mixture is stirred overnight at RT.

Working Up According to Method 1

Tetramethylethylenediamine (10-50 equivalents) is added and the mixture is stirred for 48 h at RT. Dilute NaHCO₃ solution is added, the aqueous phase is extracted exhaustively with EtOAc, and the combined organic phases are washed with NaHCO₃, water and saturated saline solution, dried (MgSO₄), filtered and freed from the solvent using the rotary evaporator. The residue is optionally purified by chromatography.

Working Up According to Method 2

The pH is adjusted to 1 with 2 N HCl and the mixture is stirred for 2 h at RT, then neutralised with 1 N NaOH, the product is isolated by extraction with CH₂Cl₂ and optionally purified by chromatography.

The following intermediate compounds are prepared according to GWM X.

| # | structure | educt |
|---|---|---|
| XXXII.1 | 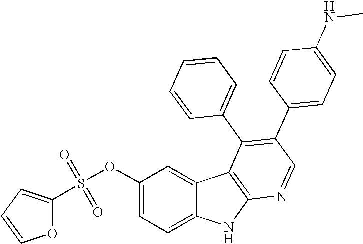 | XXX.1 |
| XXXII.2 | 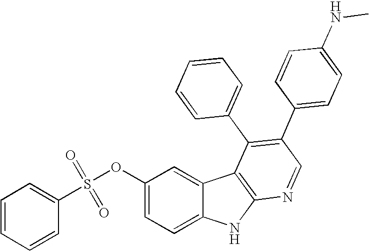 | XXX.2 |
| XXXII.3 | 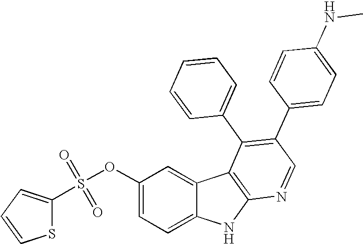 | XXX.3 |
| XXXII.4 | 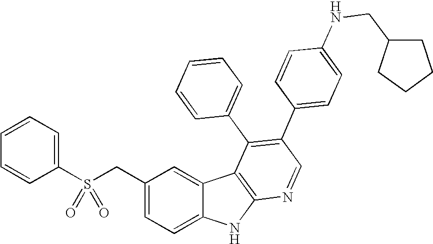 | XXXI.2 |
| XXXII.5 | 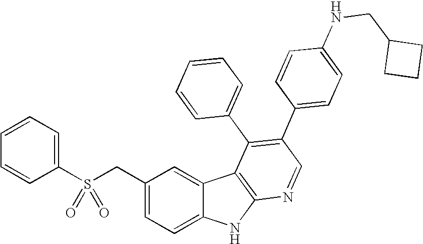 | XXXI.7 |
| XXXII.6 | 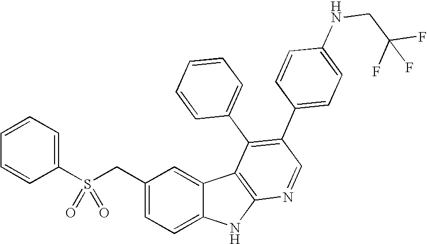 | XXXI.6 |
| XXXII.7 | 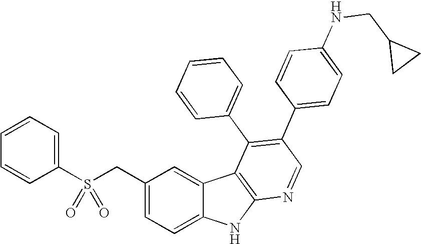 | XXXI.5 |
| XXII.8 | 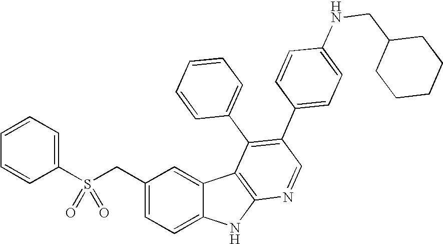 | XXXI.4 |
| XXXII.9 | 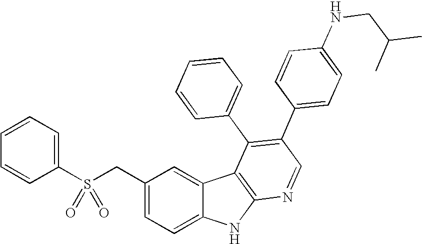 | XXXI.3 |
| XXXII.10 | 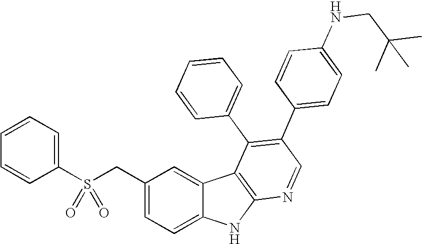 | XXXI.1 |

### Formation of Carboxamides and Sulphonamides (GWM Y)

Method 1 Starting from Acid Chlorides or Anhydrides

The acid chloride or the anhydride (1.1-5 equivalents) in substance or as a solution in anhydrous CH₂Cl₂ and then a base (triethylamine, pyridine, N-ethyldiisopropylamine or potassium carbonate; 3-50 equivalents) are added successively to a solution of the primary or secondary amine in anhydrous CH₂Cl₂ (10-100 mL/g educt) and the mixture is stirred for 1-12 h at RT. The reaction solution is diluted with CH₂Cl₂, washed with water, saturated ammonium chloride solution, saturated NaHCO₃ solution and saturated saline solution, dried (Na₂SO₄), filtered, freed from the solvent using the rotary evaporator and the crude product is optionally purified by chromatography.

Method 2 Starting from Carboxylic Acids Using TBTU

A solution of amine, carboxylic acid (1 equivalent), TBTU (1.2 equivalents) and a base (triethylamine, N-ethyldiisopropylamine or pyridine; 1-5 equivalents) in anhydrous DMF (10-20 mL/g amine) are stirred for 2-24 h at RT. Further carboxylic acid and TBTU are metered in if necessary. The reaction solution is freed from the solvent using the rotary evaporator, the residue is taken up in CH₂Cl₂, washed with water, saturated ammonium chloride solution, saturated NaHCO₃ solution and saturated saline solution, dried (Na₂SO₄), filtered, freed from the solvent using the rotary evaporator and the crude product is optionally purified by chromatography.

The following intermediate compounds are prepared according to GWM Y.

| # | structure | educt |
|---|---|---|
| XXXIII.1 | 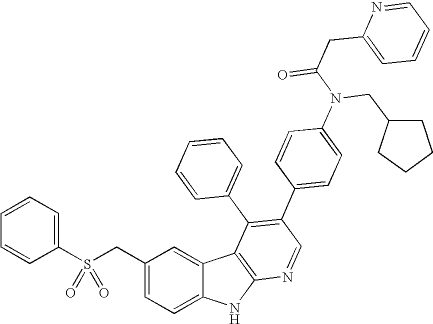 | XXXII.4 |
| XXXIII.2 | 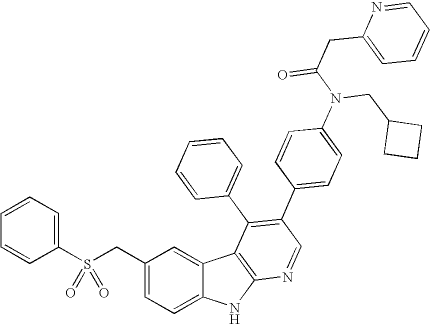 | XXXII.5 |
| XXXIII.3 | 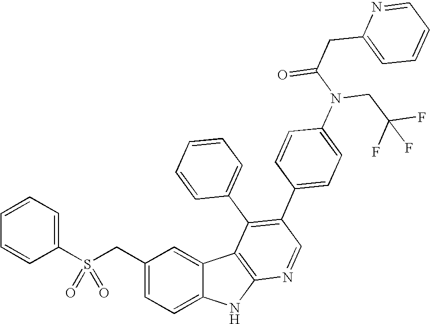 | XXXII.6 |
| XXXIII.4 | 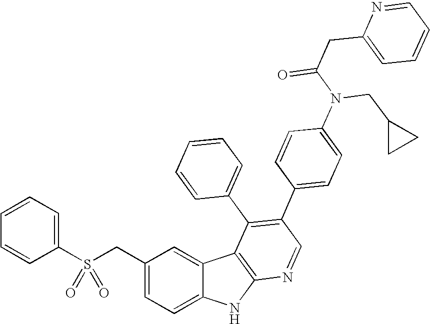 | XXXII.7 |
| XXXIII.5 | 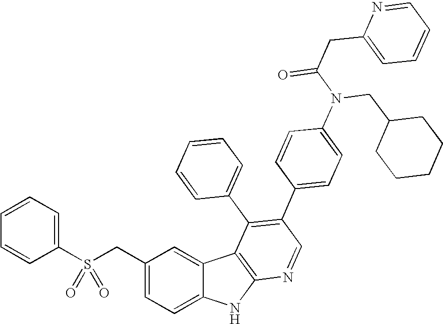 | XXXII.8 |
| XXXIII.6 | 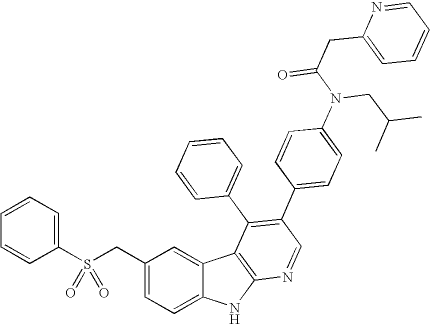 | XXXII.9 |
| XXXIII.7 | 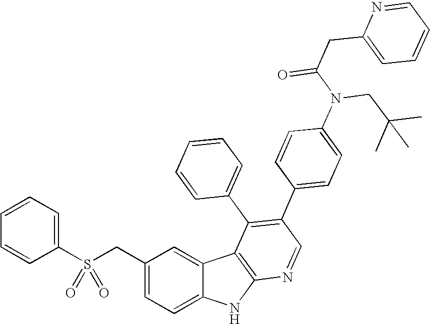 | XXXII.10 |

### Reaction of carboline-ω-halic acid amides with secondary amines (GWM Z)

A mixture of educt (prepared according to GWM L/Method 1; 20-200 mg) and secondary amine (1.5-10 equivalents) are stirred in N-methylpyrrolidinone, DMF or DMA (10-50 μL/mg educt) in the microwave reactor for 5-20 min at 150° C. The reaction mixture is purified by preparative HPLC and the eluate is freed from the solvent by freeze-drying. The reaction is carried out analogously with phenols or sulphur electrophils.

### Reaction of Carbolinamines with Glycylaldehyde Dimer (GWM AA)

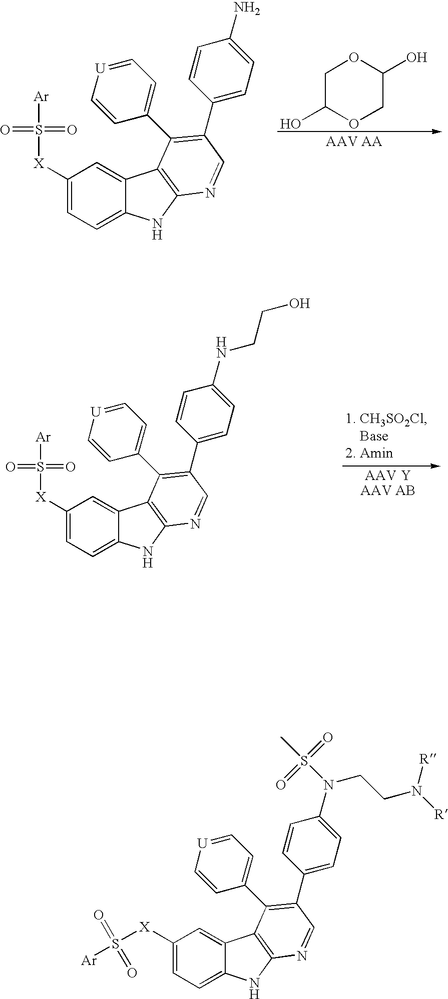

A mixture of amine, sodium cyanoborohydride (1.5 equivalents), glycylaldehyde dimer (1.5 equivalents) and ground molecular sieve (0.4 nM; 700-900 mg/mmol educt) is stirred in a mixture of anhydrous methanol and anhydrous DMF (in each case 3-5 mL/g amine) for 18-36 h at RT. If the reaction stagnates sodium cyanoborohydride and glycylaldehyde dimer are added. The suspension is diluted with saturated NaHCO₃ solution and exhaustively extracted with EtOAc. The combined organic phases are washed with saturated saline solution, dried (Na₂SO₄), filtered, freed from the solvent using the rotary evaporator and optionally purified by chromatography.

The reaction with methanesulphonic acid chloride is carried out according to GWM Y.

The following intermediate compounds are prepared analogously.

| # | structure | educt |
|---|---|---|
| XXXIV.1 | 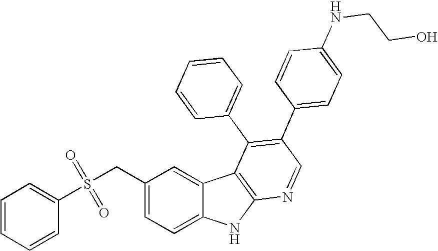 | XXI.1 |
| XXXIV.2 | 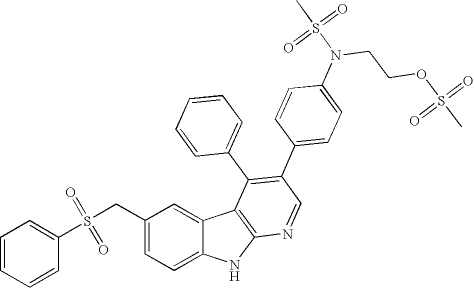 | XXXII.1 |

### Reaction to Aminoethyl-Substituted Aminocarbolines (GWM AB)

A mixture of the corresponding starting compound and the secondary amine (5-10 equivalents) in anhydrous DMF (4-10 mL/g educt) are stirred for 4-16 h at 60-100° C. and freed from the solvent using the rotary evaporator. The residue is purified by chromatography.

The following compounds are prepared according to GWM Z.

| | | tᵣₑₜ | mass |
|---|---|---|---|
| # | structure | [min] | [M + H] |
| 217 | 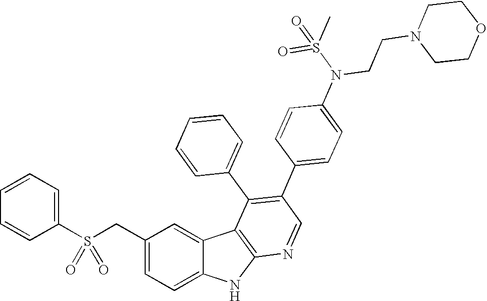 | 3.17 | 681 |
| 220 | 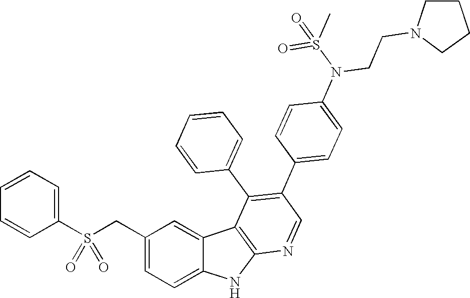 | 3.18 | 665 |
| 221 | 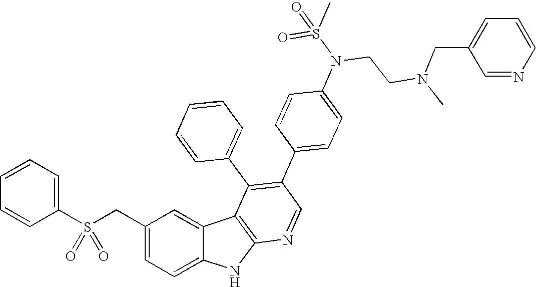 | 3.15 | 716 |
| 222 | 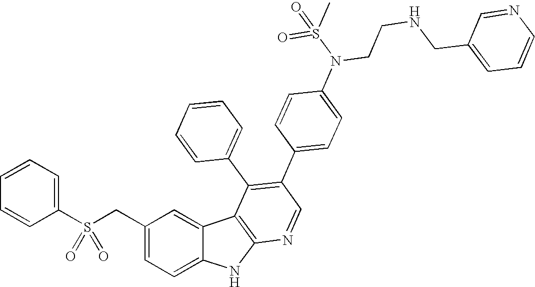 | 3.10 | 702 |

### Diazotisation and Boiling to Obtain the Phenol (GWM AC)

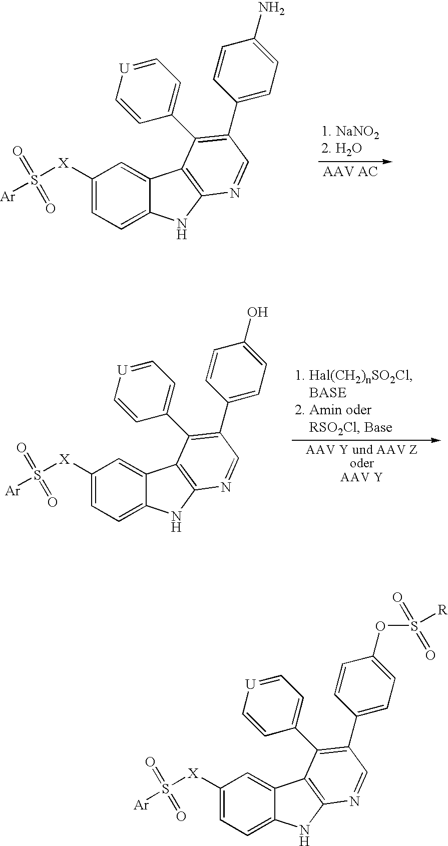

Concentrated sulphuric acid (3.5 equivalents) is added to a solution or suspension of the amine in acetic acid (20-30 mL/g amine) and the mixture is cooled to 0° C. A solution of sodium nitrite (3 equivalents) in water, saturated at 0° C., is added dropwise at 0° C. and the mixture is stirred for 2 h at this temperature. Excess nitrite is destroyed with urea. Water is added and the diazonium salt is boiled for 10-16 h at 100° C. The product is precipitated with water and obtained by filtration.

The reaction of the phenol to form the phenyl sulphonate is carried out analogously to GWM Y.

| # | structure | educt |
|---|---|---|
| XXXV.1 | 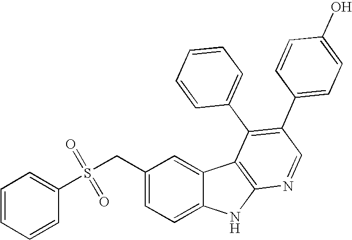 | analogously to XXIX.2 |
| XXXV.2 | 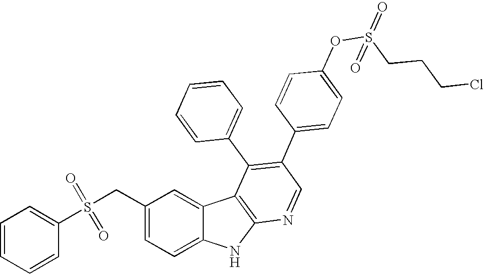 | XXXV.1 |

The reaction of halogen-substituted phenyl sulphonates to obtain the corresponding amino derivatives is carried out according to GWM Z.

### Sonogashira Coupling (GWM AD)

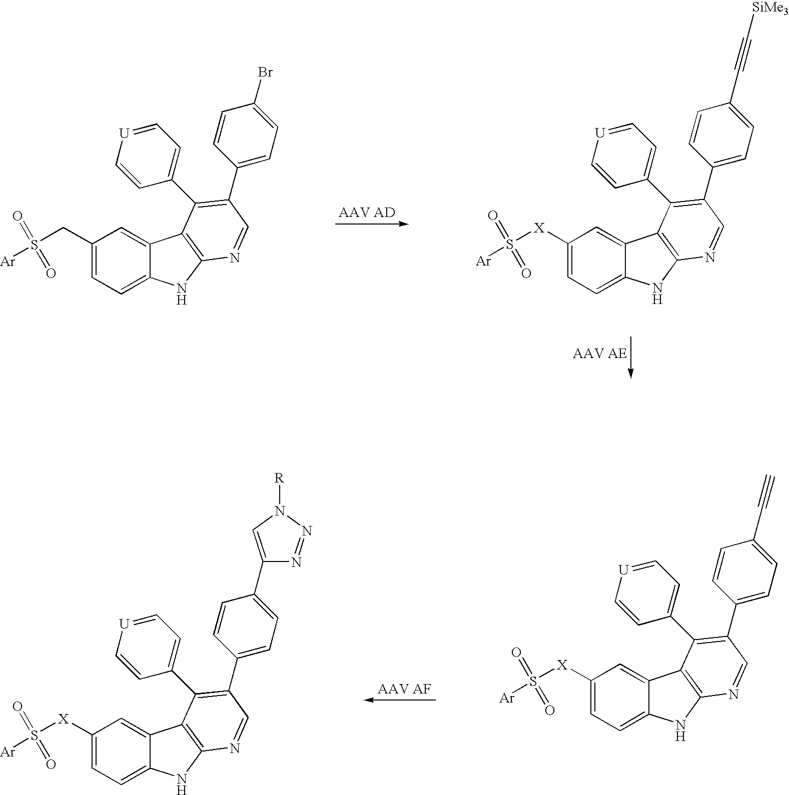

A mixture of bromine compound, bis(triphenylphosphine)palladium(II)chloride (0.1 equivalents), copper(I)iodide (0.1 equivalents), trimethylsilylacetylene (1.1 equivalents), triphenylphosphine (0.2 equivalents) and diethylamine (15-20 equivalents) in anhydrous DMF (5-15 mL/g bromine compound) are stirred for 25 min at 125° C. in the microwave reactor under argon. The mixture is freed from the solvent using the rotary evaporator and the residue is purified by chromatography.

| # | structure | educt |
|---|---|---|
| XXXVI.1 | 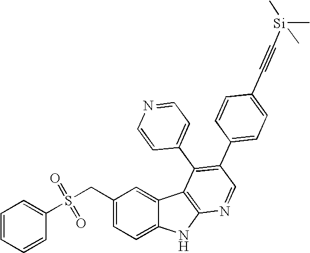 | XXI.3 |

### Cleaving of the Trimethylsilyl Protecting Group (GWM AE)

A solution of the trimethylsilylacetylene derivative in methanol (20-100 mL/g educt) is combined with 1 N potassium hydroxide (5-50 equivalents) and stirred for 24-72 h at 15-55° C. The product is isolated by filtration or extraction and optionally purified by chromatography.

| # | structure | educt |
|---|---|---|
| XXXVII.1 | 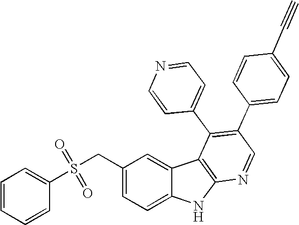 | XXXVI.1 |

### Cycloaddition to Obtain the Triazole (GWM AF)

A mixture of acetylene and azide component (1 equivalent) in water/tert-butanol (in each case 25-50 mL/g acetylene component) is combined with freshly prepared 1 M sodium-L-ascorbate solution (0.1 equivalents) and copper(II)sulphate (0.01 equivalents) and stirred for 12-24 h at 70-80° C. If the reaction stagnates further azide, sodium-L-ascorbate solution and copper(II)sulphate are metered in. The product is precipitated by adding water, isolated by filtration or extraction and optionally purified by chromatography.

The azides needed which are known from the literature may be obtained according to the following references.

| structure | Reference |
|---|---|
| 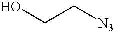 | Pfaendler et al., V. Synthesis 1996, 11, 1345-1349. |
| 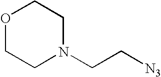 | analogously to Pfaendler et al., Synthesis 1996, 11, 1345-1349. |
| 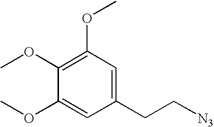 | Kita et al., J. Am. Chem. Soc. 1994, 116(9), 3684-3691 |

### Reaction of Bromophenylcarbolines to Form the Corresponding Carboxylic Acid Esters (GWM AG)

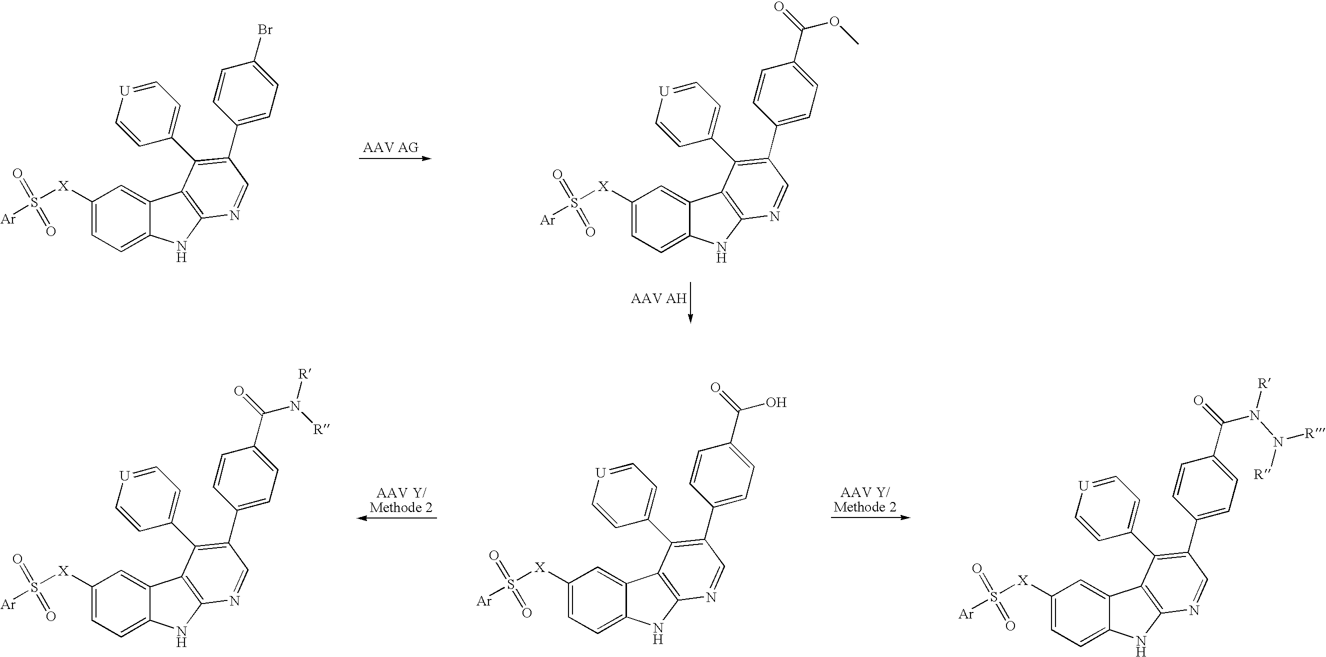

tert-Butyllithium (4 equivalents) is added to a solution of the bromine compound in anhydrous THF (50-100 mL/g educt) under argon at −78° C. and stirred for 20 min at this temperature. Then anhydrous dimethylcarbonate (2-5 equivalents) is added and the mixture is stirred for 3 h. Methanol and water are added and the mixture is extracted exhaustively with CH₂Cl₂. The combined organic phases a re washed with water and saturated saline solution, dried (Na₂SO₄), filtered, freed from the solvent using the rotary evaporator and optionally purified by chromatography.

| # | structure | educt |
|---|---|---|
| XXXVIII.1 | 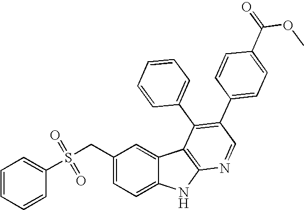 | XXI.6 |

### Ester Cleaving on Carboline Derivatives (GWM AH)

1 N aqueous LiOH solution (10 equivalents) is added at RT to a solution of the biarylcarboline ester in DMF, THF, methanol or a mixture of these solvents (10-60 mL/g ester) and the mixture is stirred for 12-48 h. It is optionally diluted with 1 N LiOH, washed with Et₂O or EtOAc, the aqueous phase is acidified with 2 N HCl, the precipitated carboxylic acid is recovered by extraction or filtration and the crude product is optionally purified by column chromatography.

| # | structure | educt |
|---|---|---|
| IXL.1 | 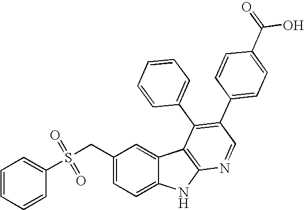 | XXXVIII.1 |
| IXL.2 | 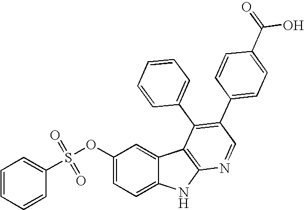 | Analogously to XXXVIII.1 |
| IXL.3 | 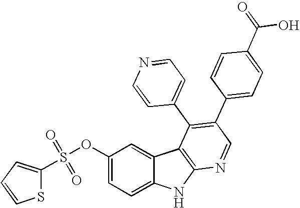 | Analogously to XXXVIII.1 |

The reaction of the carboxylic acids with substituted amines to form amides or with substituted hydrazine derivatives to form hydrazides is carried out according to GWM L,

Method 2, using TBTU. Trimethylhydrazine may be obtained according to the method of Ankersen et al. (Eur. J. Med. Chem. 2000, 35(5), 487-497).

Examples 174-337 are prepared according to GWM N-AH.

| | | tᵣₑₜ | mass |
|---|---|---|---|
| # | structure | [min] | [M + H] |
| 174 | 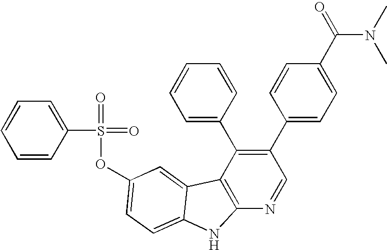 | 3.35 | 548 |
| 175 | 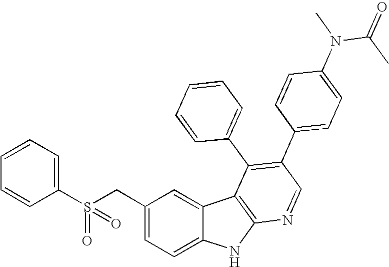 | 3.19 | 546 |
| 176 | 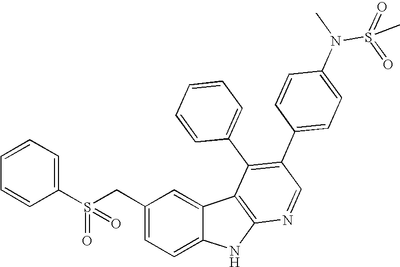 | 4.02 | 582 |
| 177 | 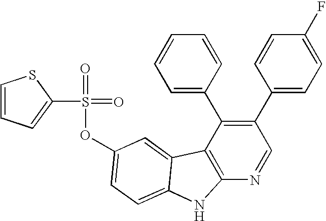 | 3.65 | 501 |
| 178 | 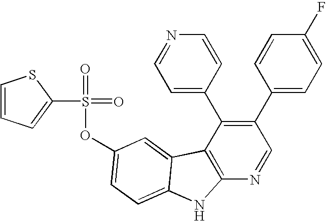 | 3.17 | 502 |
| 179 | 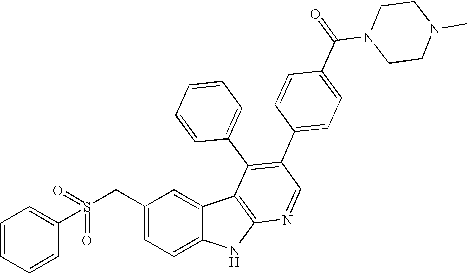 | 2.58 | 601 |
| 180 | 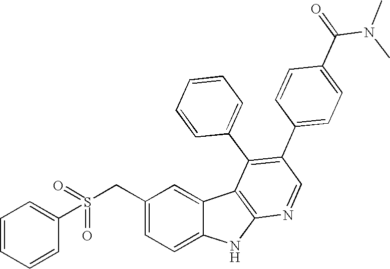 | 3.08 | 546 |
| 181 | 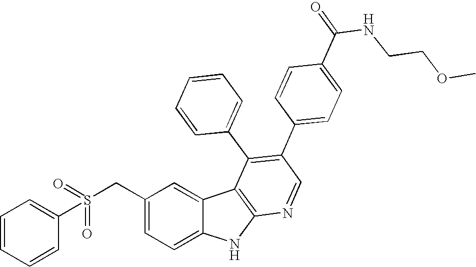 | 3.04 | 576 |
| 182 | 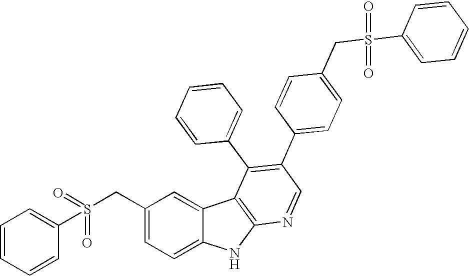 | 3.06 | 629 |
| 183 | 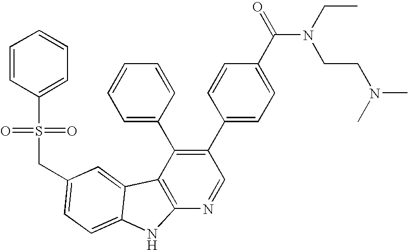 | 2.66 | 309 [M + 2H]²⁻ |
| 184 | 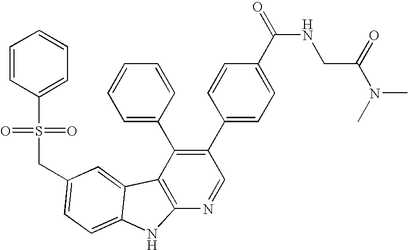 | 2.96 | 603 |
| 185 | 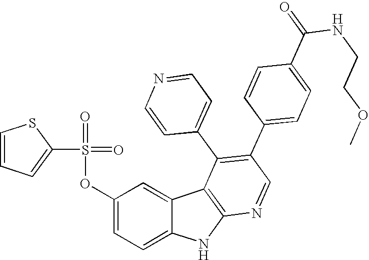 | 2.82 | 585 |
| 186 | 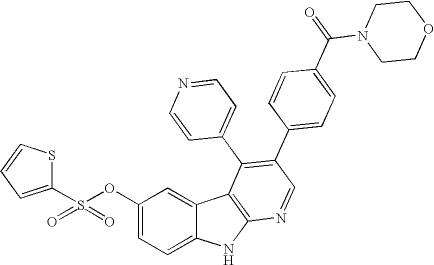 | 2.86 | 597 |
| 187 | 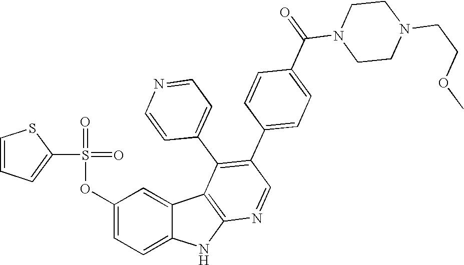 | 2.52 | 654 |
| 188 | 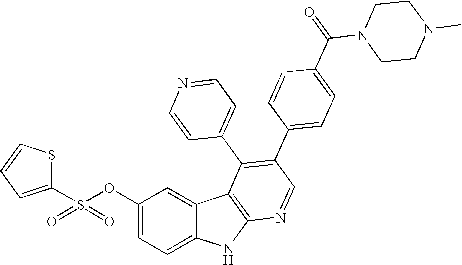 | 2.52 | 610 |
| 189 | 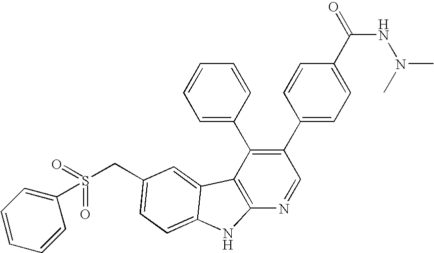 | 2.85 | 559 [M + 2H]²⁻ |
| 190 | 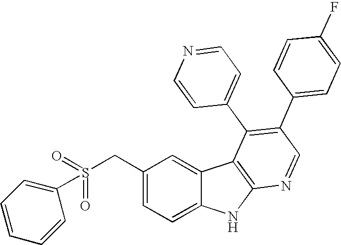 | 2.93 | 494 |
| 191 | 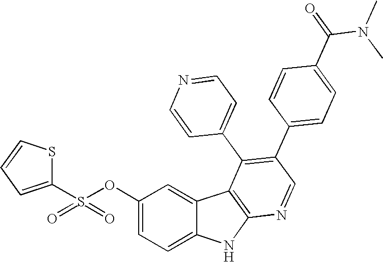 | 2.83 | 555 |
| 192 | 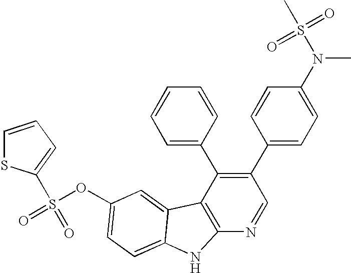 | 4.31 | 590 |
| 193 | 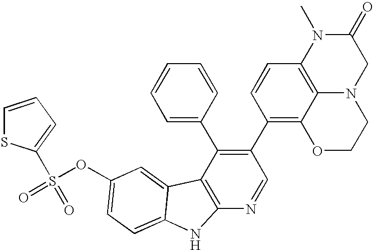 | 3.34 | 639 |
| 194 | 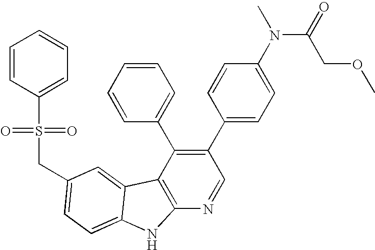 | 3.78 | 576 |
| 195 | 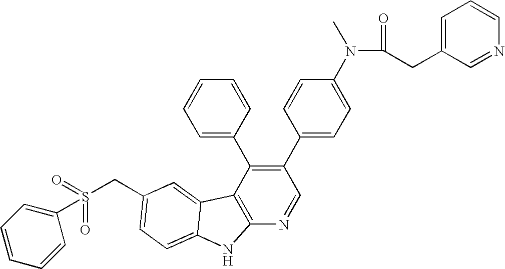 | 3.36 | 623 |
| 196 | 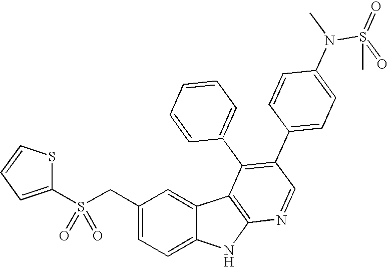 | 4.01 | 588 |
| 197 | 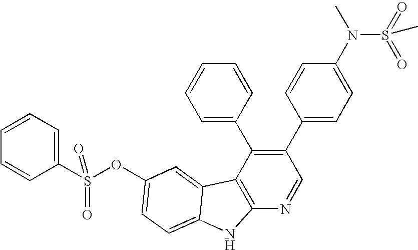 | 4.31 | 584 |
| 198 | 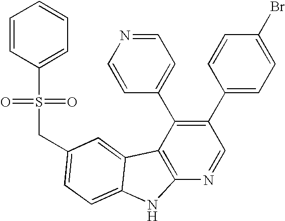 | 3.85 | 555 |
| 199 | 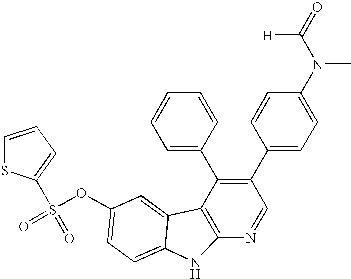 | 4.16 | 540 |
| 200 | 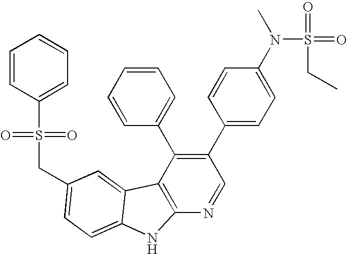 | 4.15 | 596 |
| 201 | 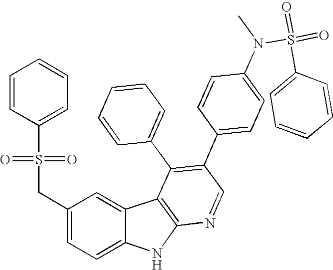 | 4.47 | 645 |
| 202 | 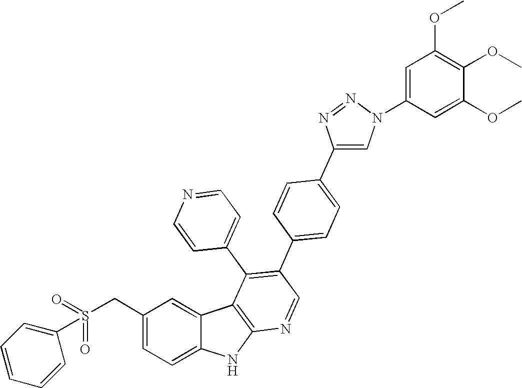 | 3.88 | 709 |
| 203 | 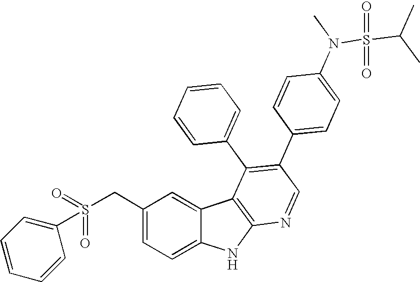 | 4.27 | 610 |
| 204 | 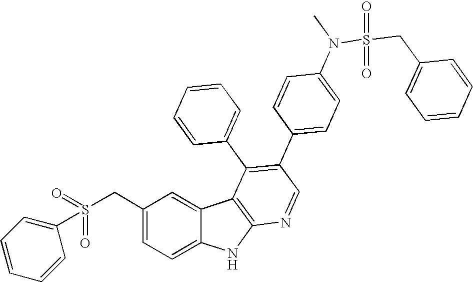 | 4.47 | 658 |
| 205 | 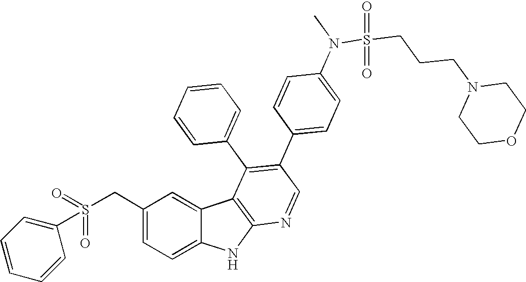 | 3.28 | 695 |
| 206 | 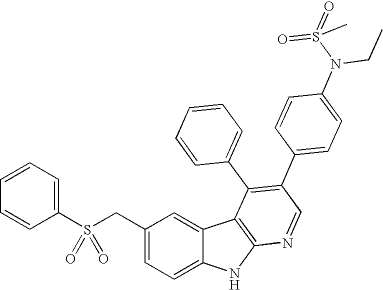 | 4.09 | 596 |
| 207 | 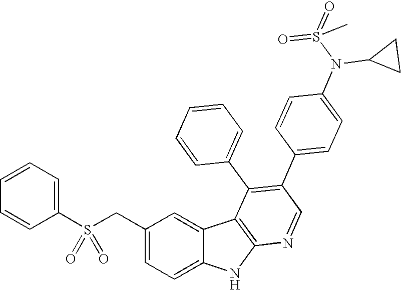 | 4.17 | 608 |
| 208 | 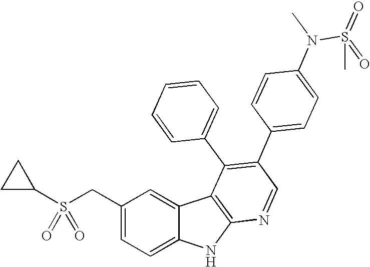 | 3.80 | 546 |
| 209 | 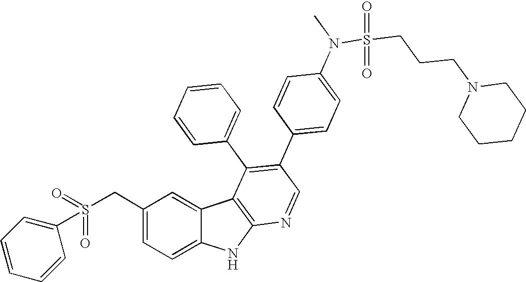 | 3.29 | 693 |
| 210 | 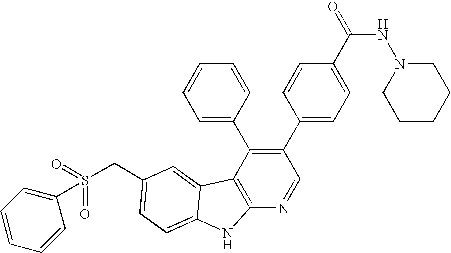 | 3.78 | 601 |
| 211 | 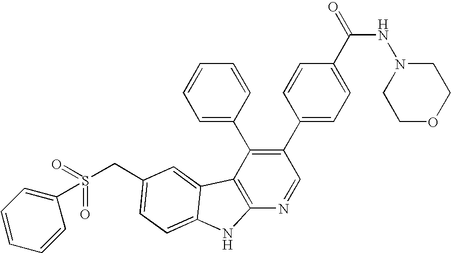 | 3.58 | 603 |
| 212 | 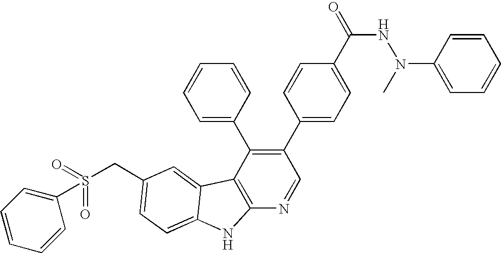 | 4.15 | 623 |
| 213 | 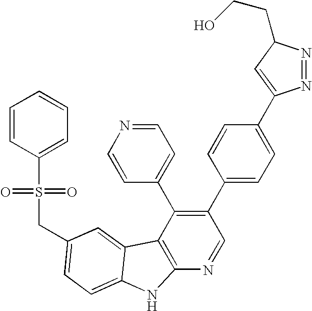 | 3.14 | 587 |
| 214 | 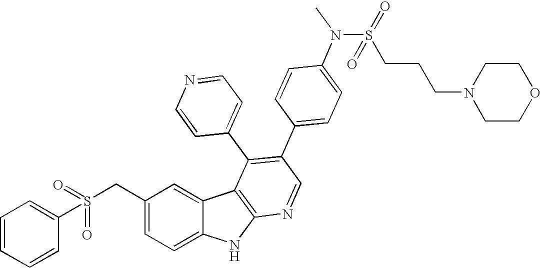 | 2.97 | 696 |
| 215 | 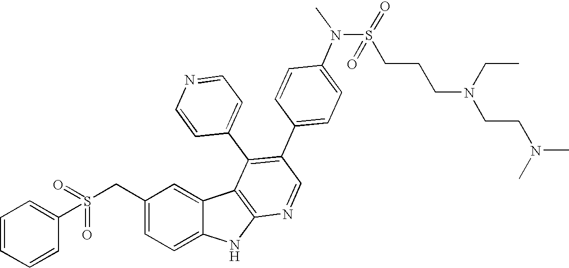 | 2.82 | 725 |
| 216 | 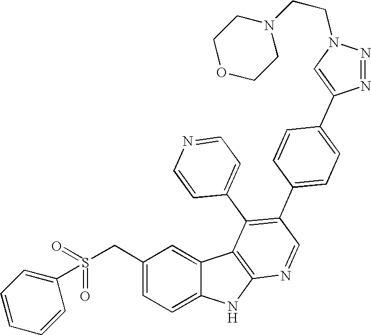 | 2.92 | 656 |
| 218 | 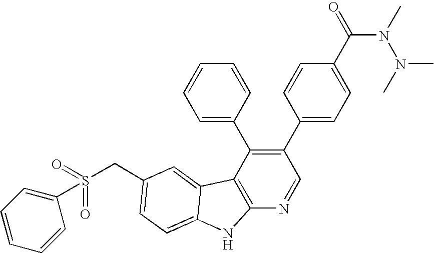 | 3.98 | 575 |
| 219 | 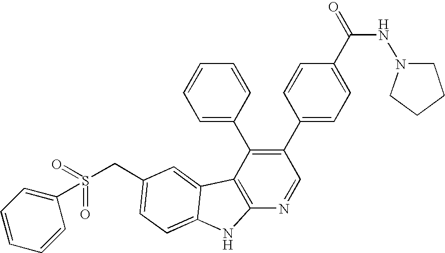 | 3.51 | 587 |
| 223 | 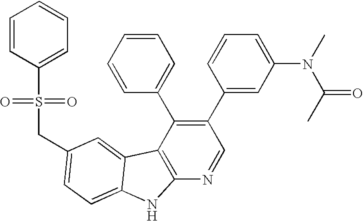 | 3.83 | 546 |
| 224 | 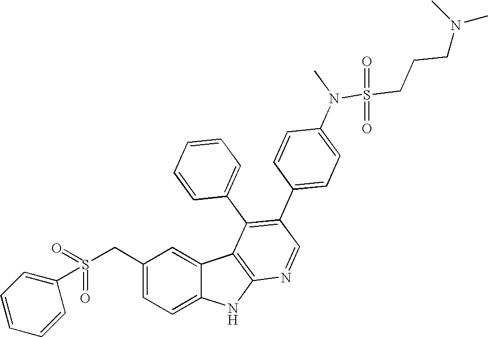 | 3.16 | 653 |
| 225 | 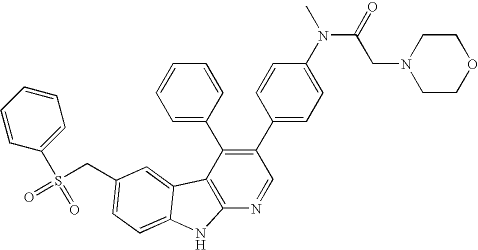 | 3.12 | 631 |
| 226 | 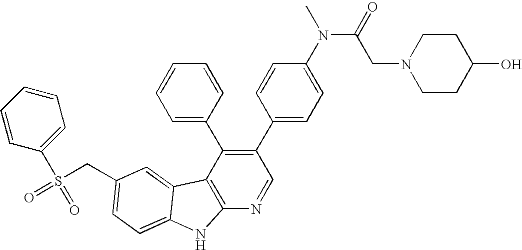 | 3.14 | 645 |
| 227 | 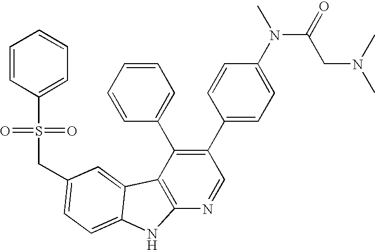 | 3.15 | 589 |
| 228 | 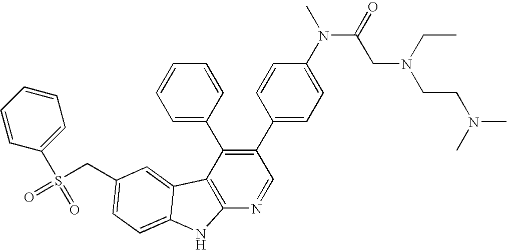 | 3.20 | 660 |
| 229 | 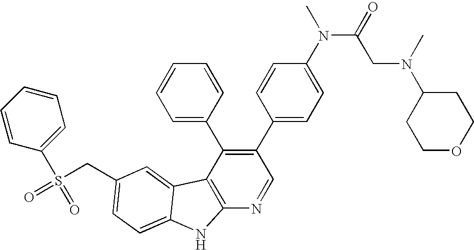 | 3.01 | 659 |
| 230 | 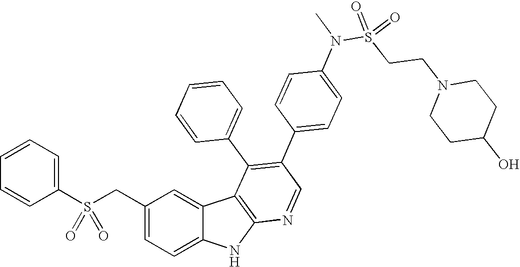 | 3.23 | 695 |
| 231 | 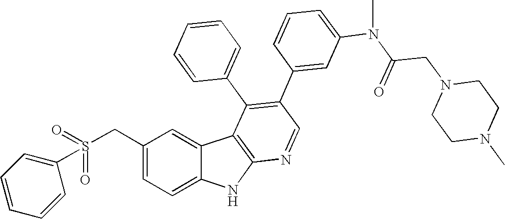 | 3.13 | 644 |
| 232 | 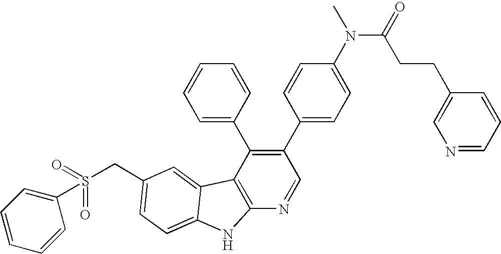 | 3.32 | 637 |
| 233 | 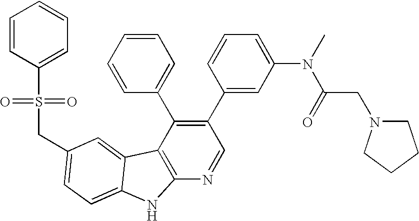 | 3.17 | 615 |
| 234 | 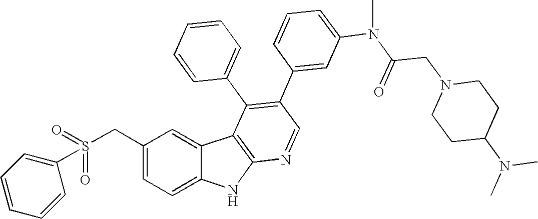 | 2.91 | 672 |
| 235 | 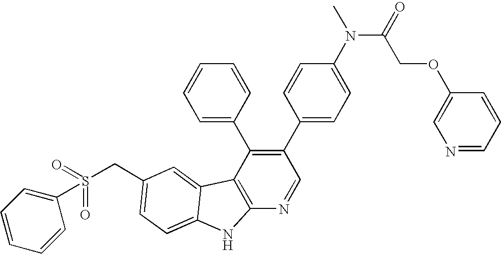 | 3.50 | 320 [M + 2H]²⁻ |
| 236 | 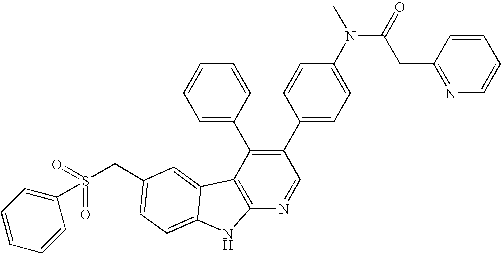 | 3.43 | 623 |
| 237 | 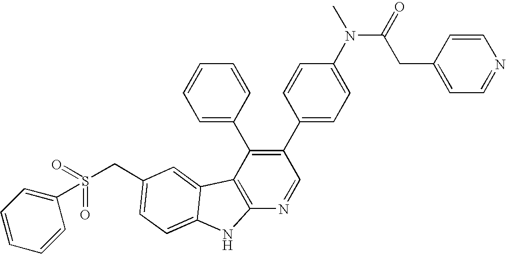 | 3.26 | 623 |
| 238 | 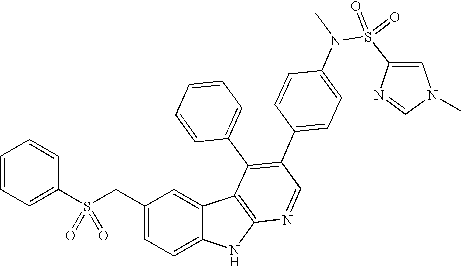 | 3.87 | 648 |
| 239 | 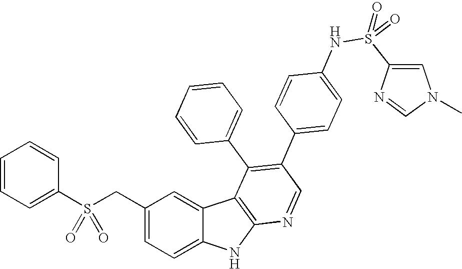 | 3.69 | 634 |
| 240 | 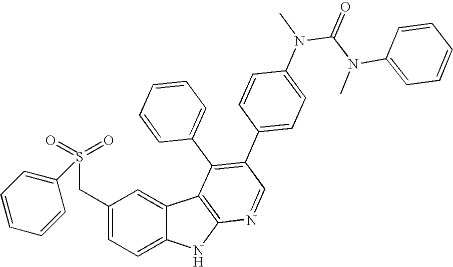 | 4.25 | 637 |
| 241 | 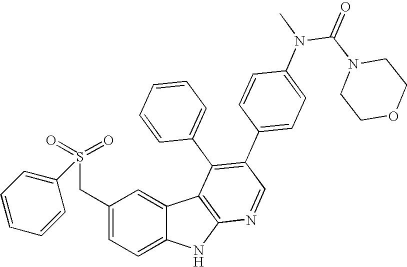 | 3.87 | 617 |
| 242 | 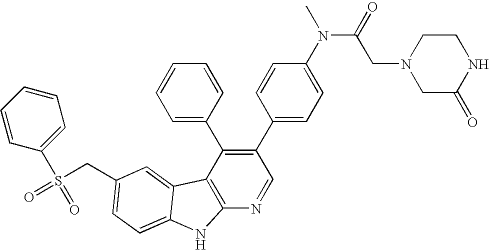 | 3.26 | 644 |
| 243 | 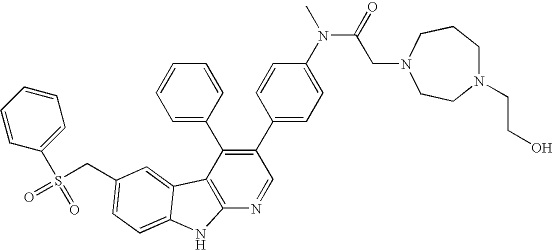 | 3.00 | 688 |
| 244 | 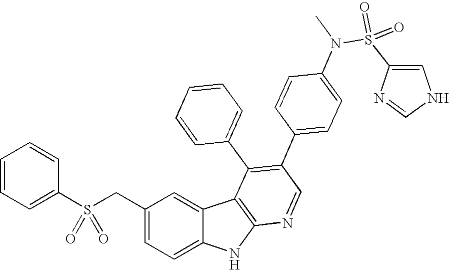 | 3.77 | 634 |
| 245 | 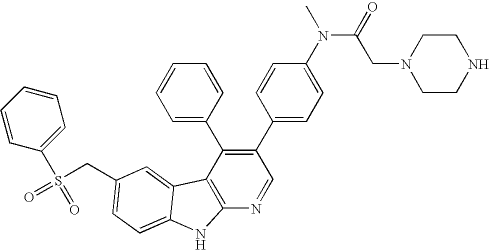 | 3.08 | 630 |
| 246 | 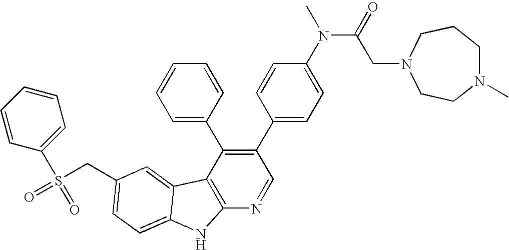 | 3.02 | 658 |
| 247 | 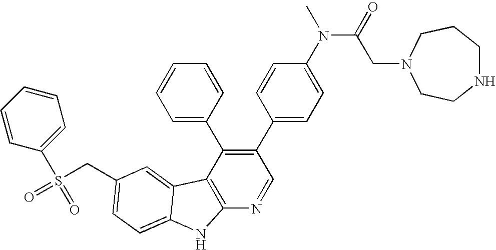 | 2.94 | 644 |
| 248 | 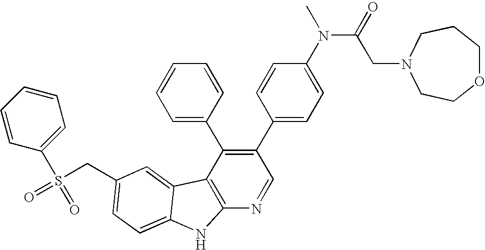 | 3.21 | 645 |
| 249 | 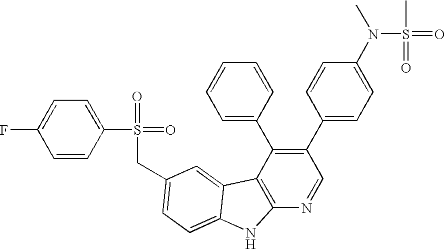 | 4.04 | 600 |
| 250 | 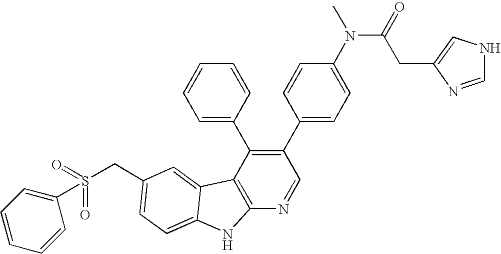 | 3.13 | 612 |
| 251 | 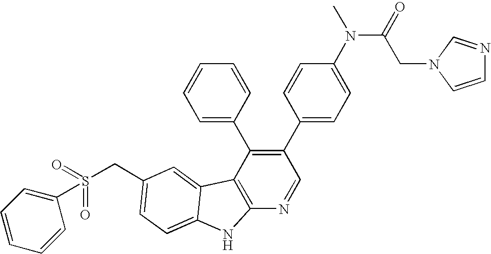 | 3.14 | 612 |
| 252 | 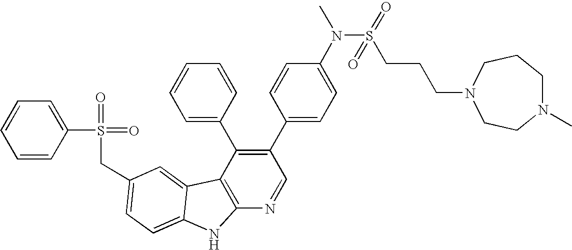 | 3.00 | 722 |
| 253 | 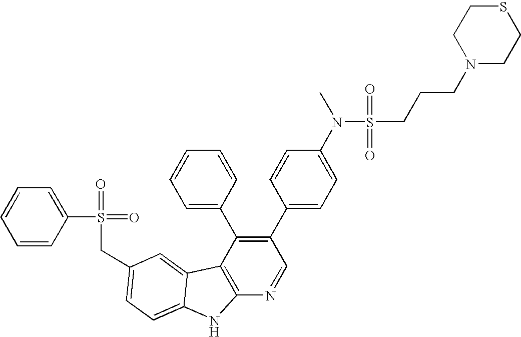 | 3.30 | 711 |
| 254 | 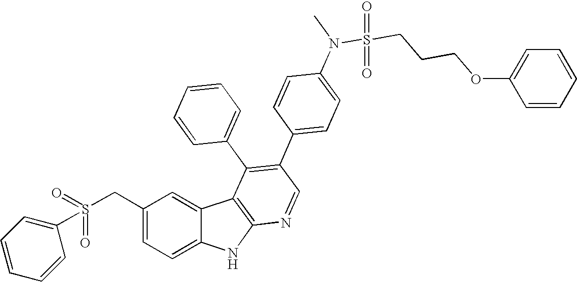 | 2.89 | 702 |
| 255 | 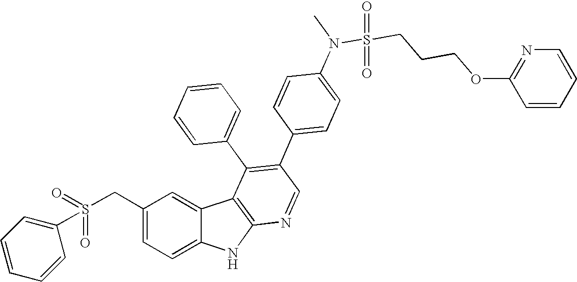 | 2.87 | 702 |
| 256 | 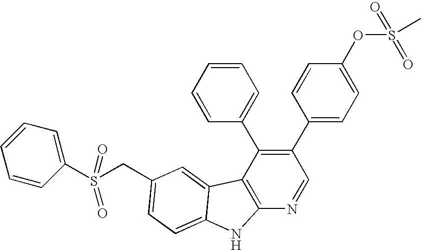 | 4.11 | 569 |
| 257 | 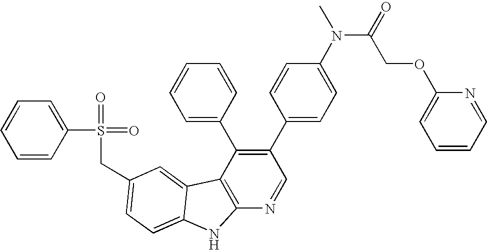 | 2.68 | 629 |
| 258 | 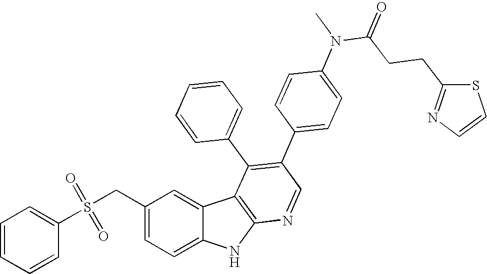 | 2.94 | 642 |
| 259 | 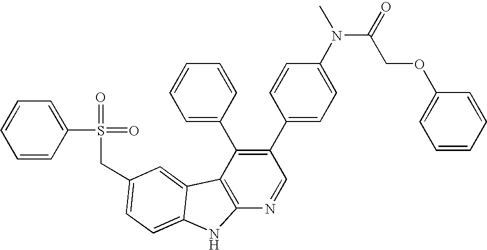 | 4.26 | 628 |
| 260 | 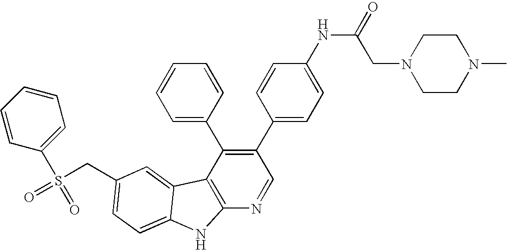 | 2.08 | 620 |
| 261 | 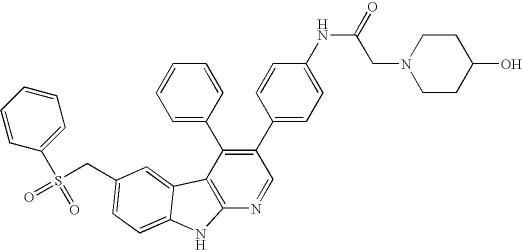 | 2.06 | 621 |
| 262 | 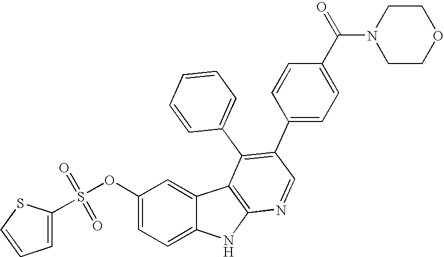 | 4.05 | 596 |
| 263 | 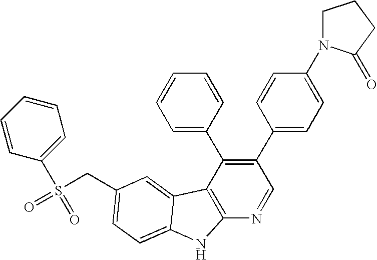 | 2.99 | 558 |
| 264 | 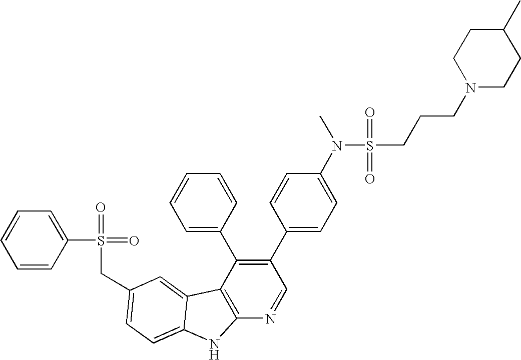 | 2.42 | 707 |
| 265 | 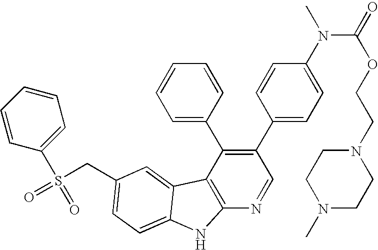 | 2.26 | 227.5 [M + 2H]²⁻ |
| 266 | 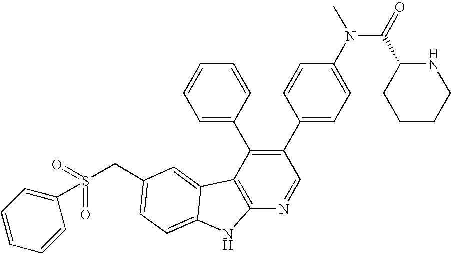 | 2.22 | 615 |
| 267 | 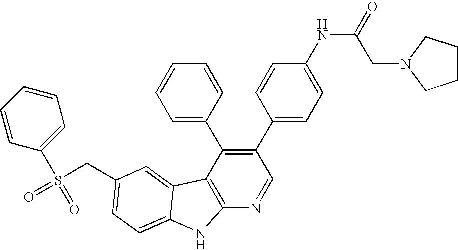 | 2.20 | 601 |
| 268 | 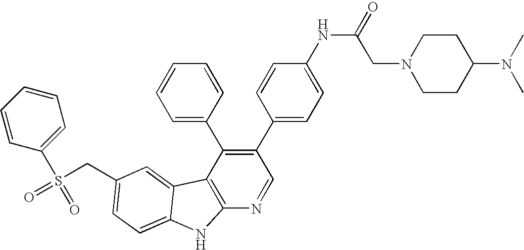 | 2.94 | 229 [M + 2H]²⁻ |
| 269 | 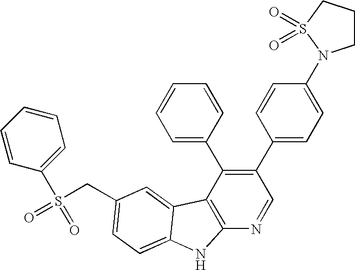 | 2.92 | 594 |
| 270 | 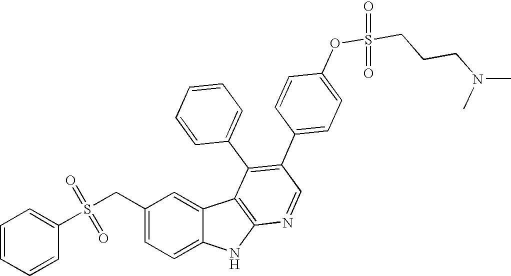 | 2.26 | 640 |
| 271 | 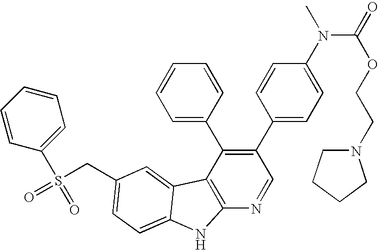 | 2.26 | 222 [M + 2H]²⁻ |
| 272 | 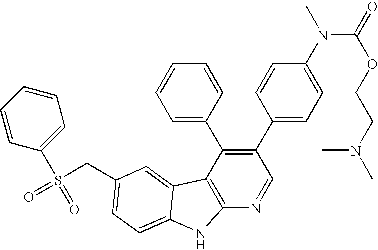 | 2.20 | 619 |
| 273 | 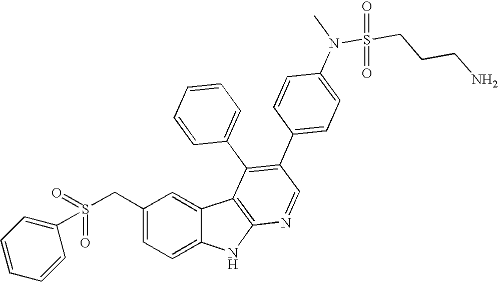 | 2.20 | 212 [M + 2H]²⁻ |
| 274 | 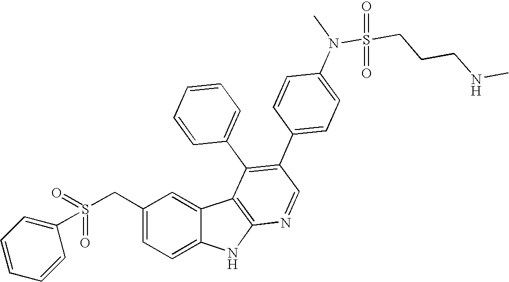 | 2.20 | 629 |
| 275 | 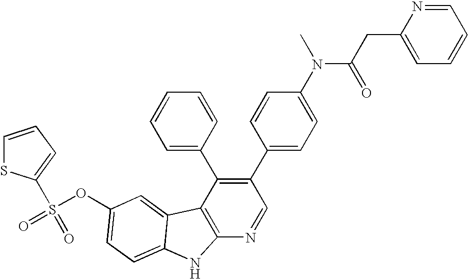 | 2.62 | 621 |
| 276 | 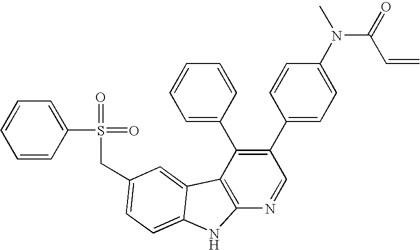 | 2.96 | 558 |
| 277 | 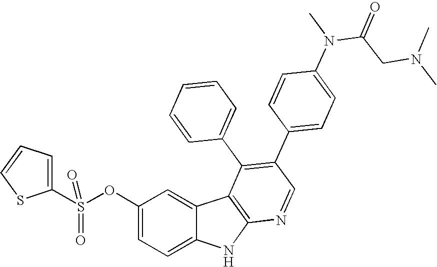 | 2.29 | 597 |
| 278 | 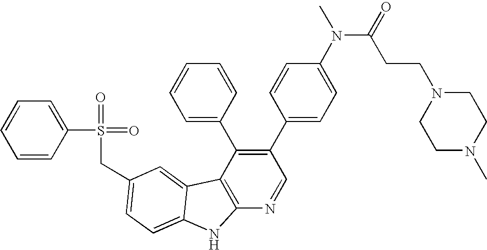 | 2.09 | 658 |
| 279 | 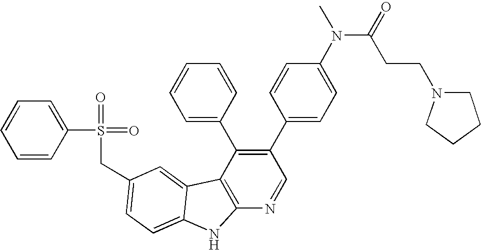 | 2.19 | 629 |
| 280 | 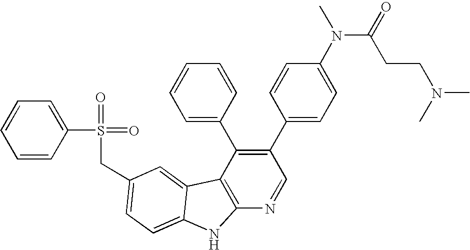 | 2.12 | 602 |
| 281 | 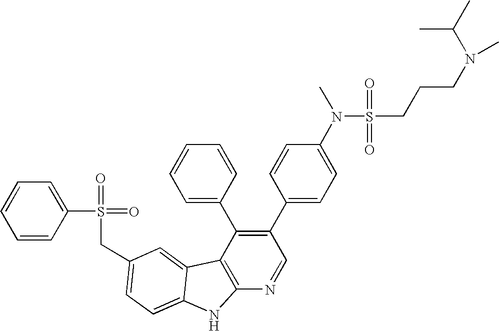 | 2.27 | 681 |
| 282 | 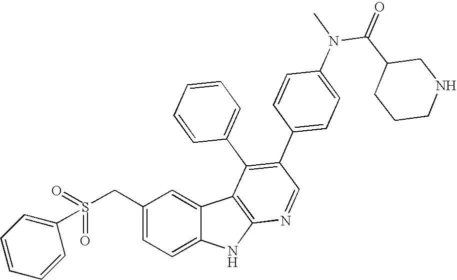 | 2.20 | 615 |
| 283 | 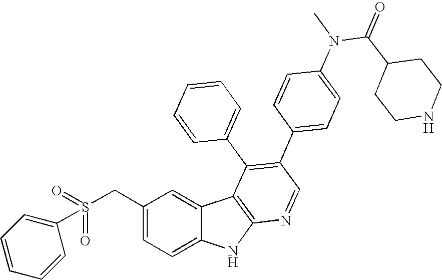 | 2.14 | 615 |
| 284 | 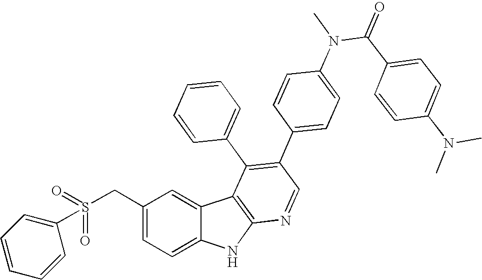 | 4.22 | 226 [M + 2H]²⁻ |
| 285 | 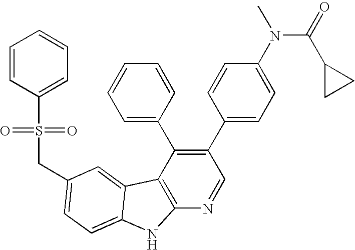 | 4.06 | 572 |
| 286 | 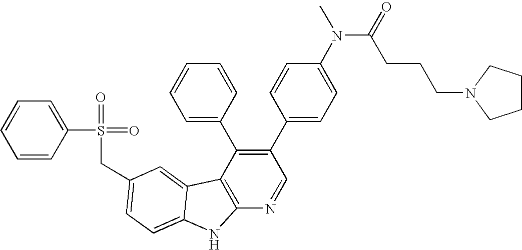 | 2.18 | 642 |
| 287 | 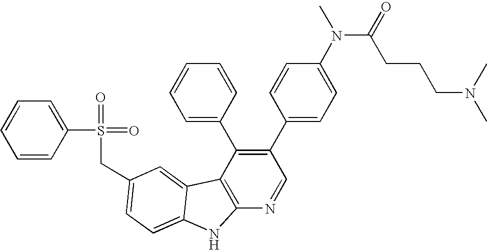 | 2.17 | 617 |
| 288 | 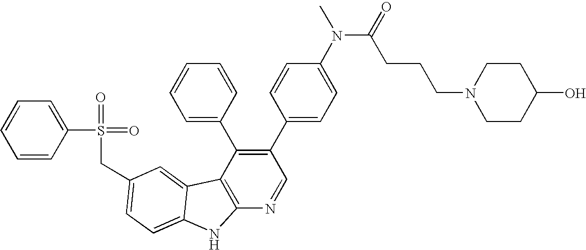 | 2.17 | 672 |
| 289 | 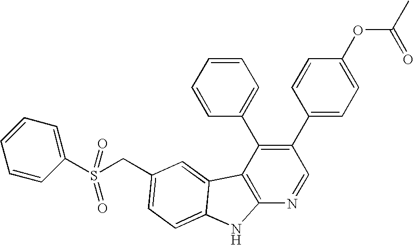 | 4.20 | 522 |
| 290 | 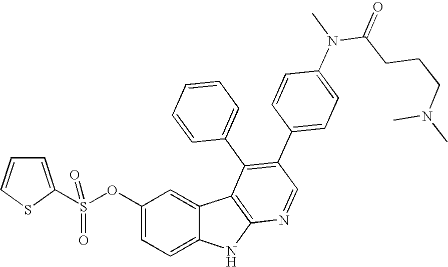 | 2.22 | 625 |
| 291 | 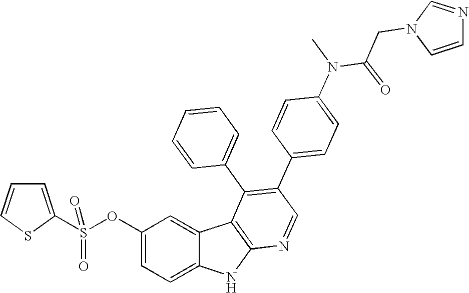 | 2.22 | 620 |
| 292 | 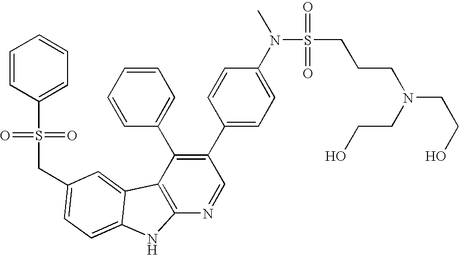 | 2.19 | 712 |
| 293 | 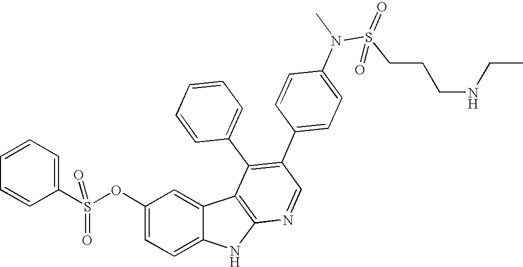 | 2.22 | 652 |
| 294 | 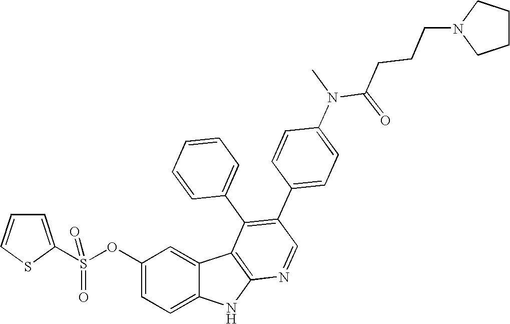 | 2.22 | 651 |
| 295 | 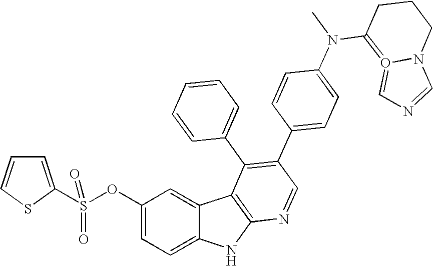 | 2.20 | 224 [M + 2H]²⁻ |
| 296 | 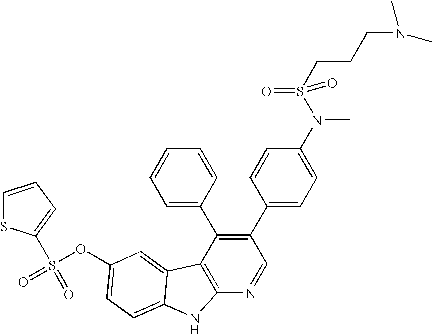 | 2.28 | 661 |
| 297 | 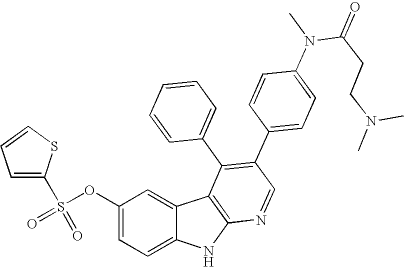 | 2.21 | 611 |
| 298 | 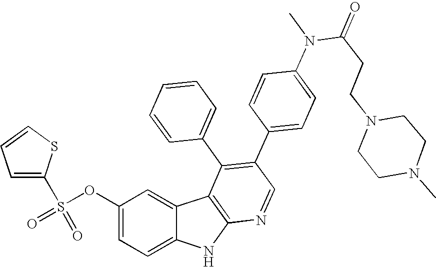 | 2.14 | 666 |
| 299 | 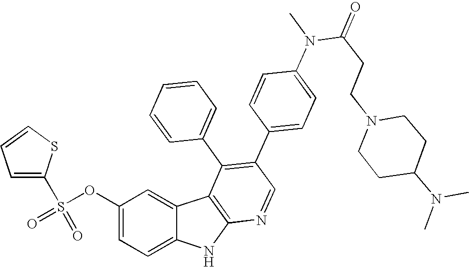 | 2.96 | 694 |
| 300 | 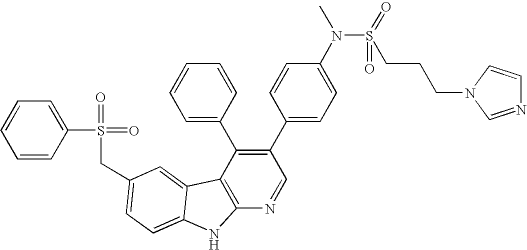 | 4.56 | 676 |
| 301 | 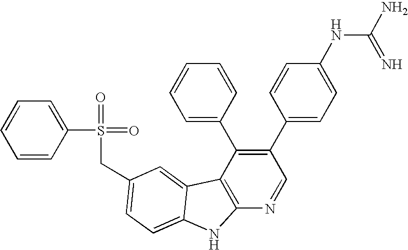 | 2.99 | 522 |
| 302 | 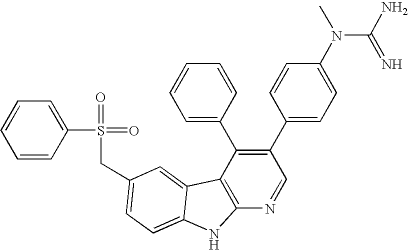 | 2.04 | 546 |
| 303 | 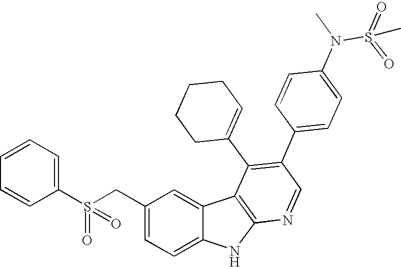 | 4.09 | 586 |
| 304 | 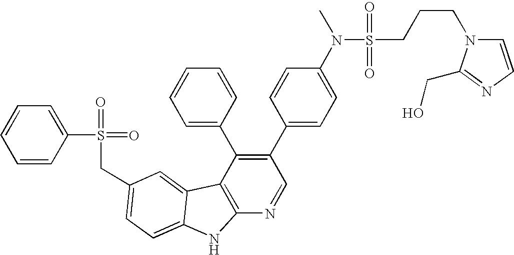 | 2.16 | 706 |
| 305 | 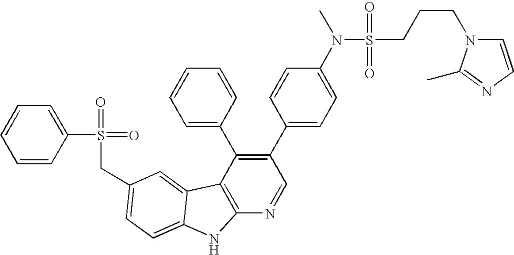 | 2.21 | 690 |
| 306 | 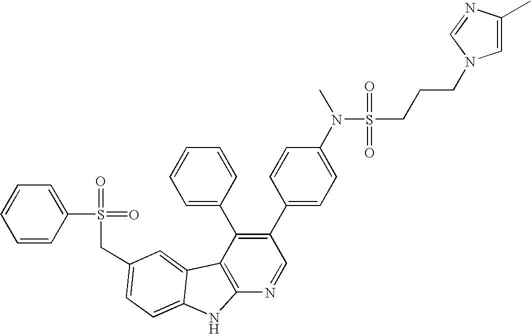 | 2.21 | 290 |
| 307 | 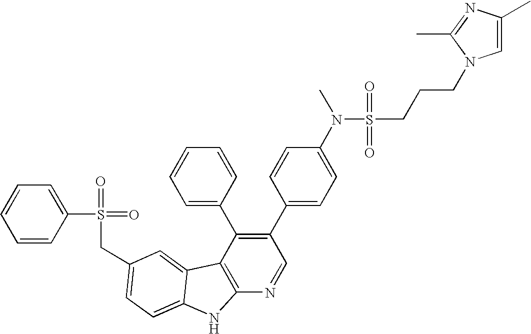 | 2.22 | 704 |
| 308 | 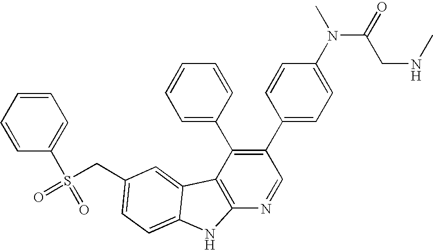 | 2.02 | 575 |
| 309 | 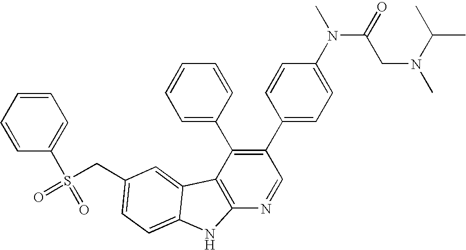 | 2.07 | 617 |
| 310 | 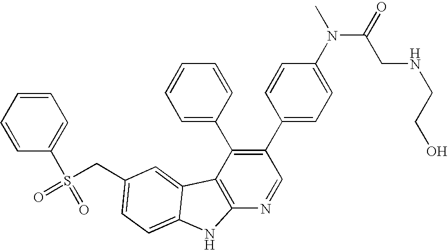 | 2.00 | 605 |
| 311 | 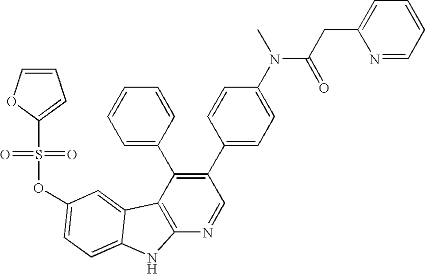 | 2.51 | 615 |
| 312 | 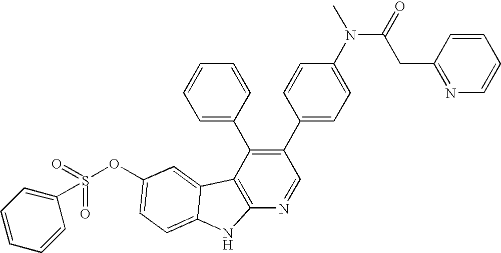 | 2.64 | 625 |
| 313 | 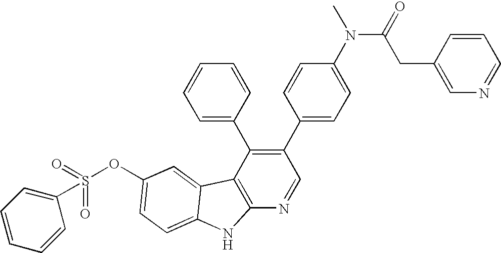 | 2.51 | 625 |
| 314 | 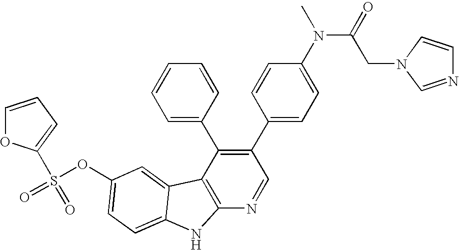 | 2.21 | 604 |
| 315 | 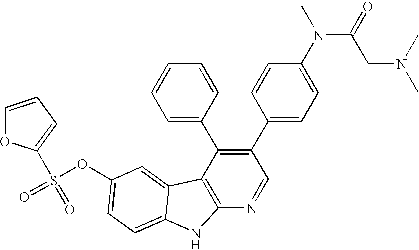 | 2.16 | 581 |
| 316 | 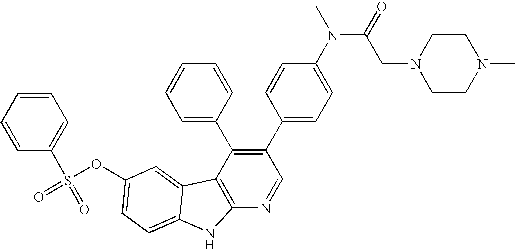 | 2.22 | 646 |
| 317 | 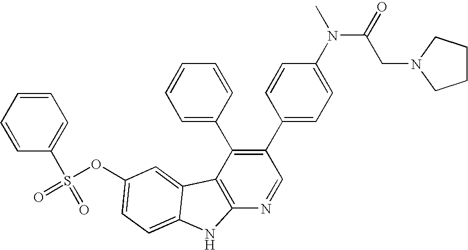 | 2.25 | 617 |
| 318 | 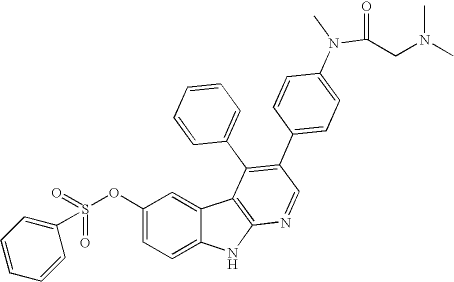 | 2.22 | 591 |
| 319 | 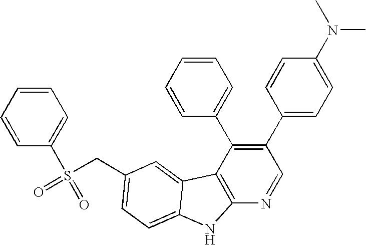 | 4.01 | 518 |
| 320 | 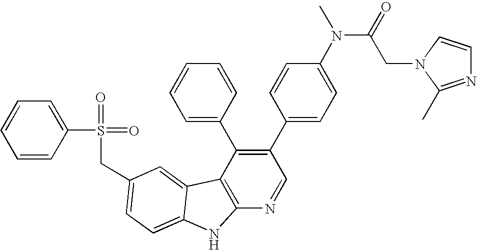 | 2.12 | 626 |
| 321 | 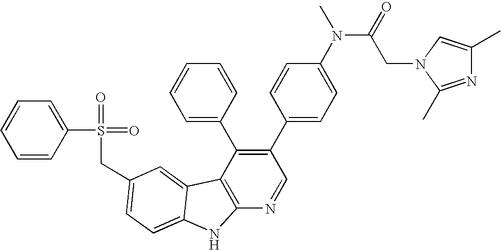 | 2.15 | 640 |
| 322 | 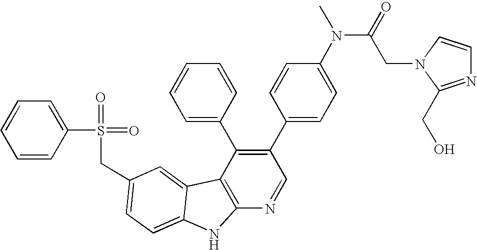 | 2.16 | 642 |
| 323 | 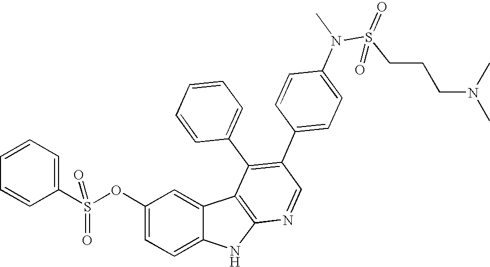 | 2.22 | 655 |
| 324 | 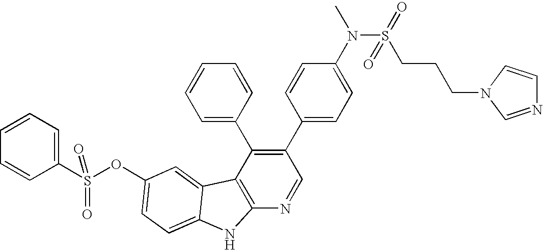 | 2.25 | 678 |
| 325 | 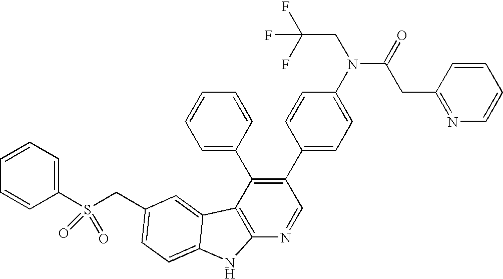 | 2.80 | 691 |
| 326 | 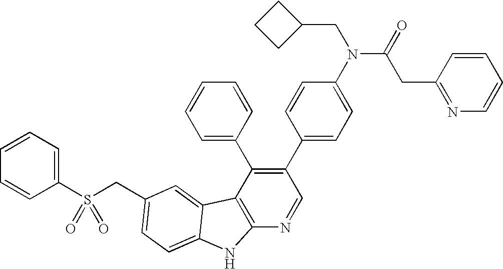 | 2.80 | 677 |
| 327 | 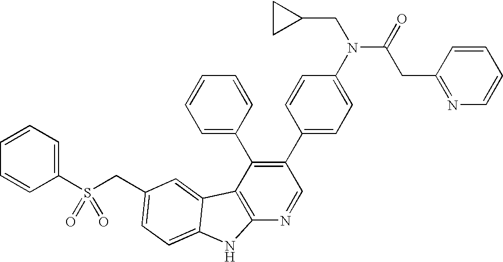 | 2.67 | 662 |
| 328 | 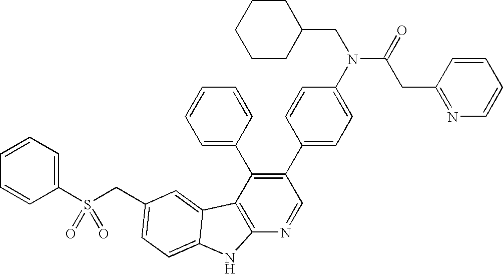 | 4.06 | 705 |
| 329 | 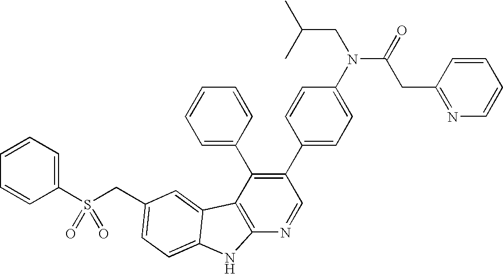 | 2.78 | 665 |
| 330 | 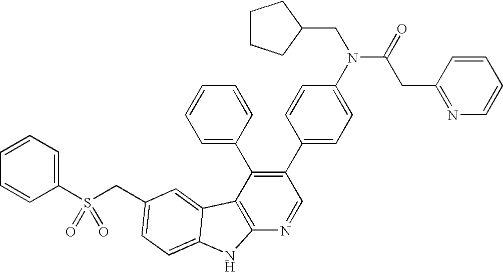 | 2.96 | 691 |
| 331 | 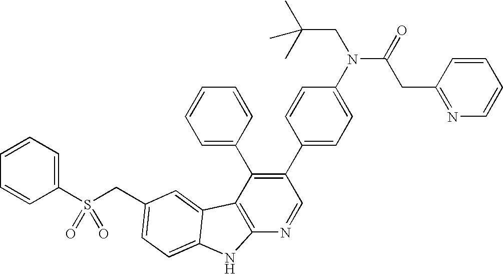 | 2.82 | 679 |
| 332 | 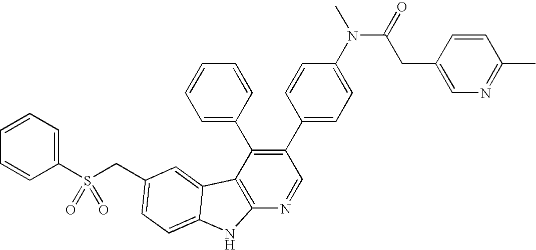 | 2.24 | 627 |
| 333 | 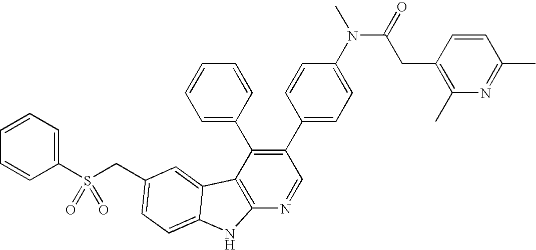 | 2.24 | 651 |
| 334 | 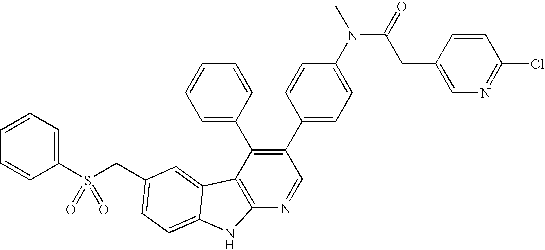 | 4.22 | 657 |
| 335 | 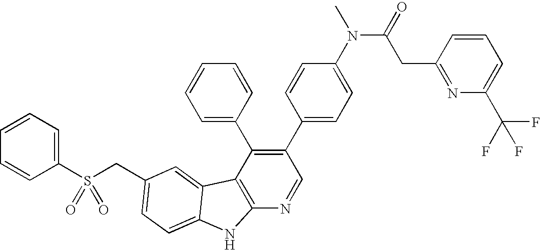 | 4.27 | 691 |
| 336 | 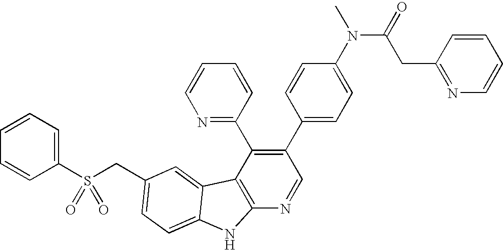 | 2.21 | 624 |
| 337 | 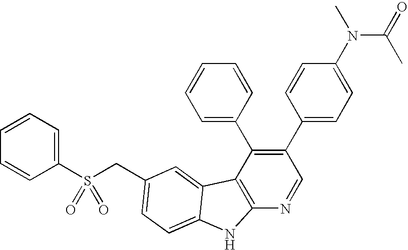 | 2.55 | 547 |

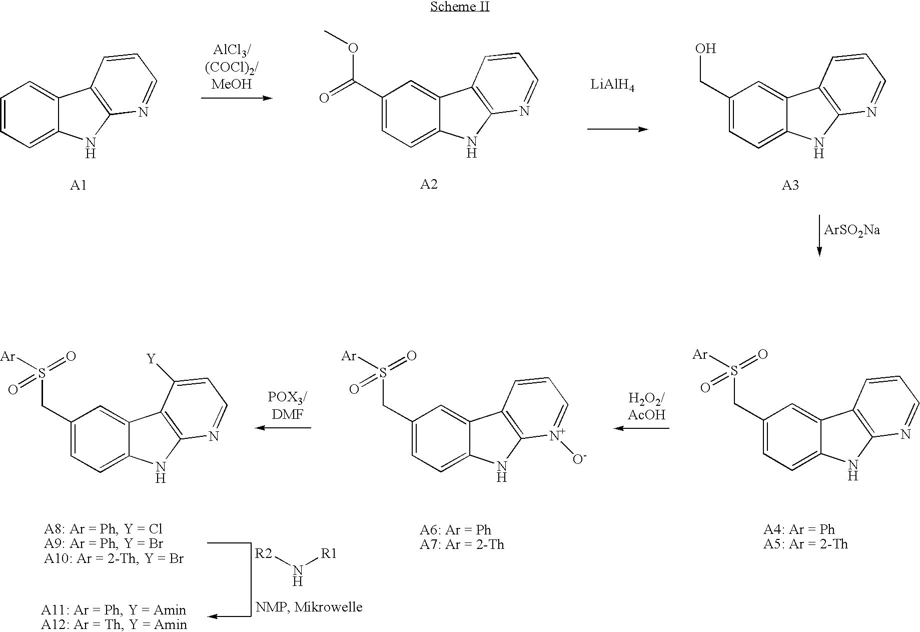

### A1) 9H-pyrido[2,3-b]indole (α-carboline)

α-Carboline (A1) is prepared according to Stephenson et al., J. Chem. Soc. C, 1970, 10, 1355-1364.

### A2) methyl 9H-pyrido[2,3-b]indol-6-carboxylate

α-Carboline (A1) (36.5 g, 217 mmol) is added at 0-5° C. to a suspension of anhydrous aluminium chloride (72.4 g, 543 mmol) in anhydrous CH₂Cl₂ (1.2 L). Oxalyl chloride (37.3 mL, 434 mmol) is added dropwise within 40 min at this temperature and the mixture is stirred for 1 h. It is poured slowly onto a cooled mixture of anhydrous CH₂Cl₂ (800 mL) and anhydrous methanol (800 mL) and stirred for 30 min. The mixture is filtered and washed with water (1 L). The aqueous phase is exhaustively extracted with CH₂Cl₂ and the filter residue is stirred out with CH₂Cl₂. The combined organic phases are washed with water (2×500 mL) and saturated saline solution (1×500 mL), dried (MgSO₄), filtered and freed from the solvent using the rotary evaporator. The residue is digested with tert-butylmethylether (2×50 mL), thus producing methyl 9H-pyrido[2,3-b]indole-6-carboxylate (A2) in the form of crystals.

### A3) 9H-pyrido[2,3-b]indole-6-methanol

Methyl 9H-pyrido[2,3-b]indole-6-carboxylate (A2) (27.7 g, 122 mmol) is added at 0-5° C. to a suspension of lithium aluminium hydride (9.29 g, 245 mmol) in anhydrous THF (600 mL)/anhydrous Et₂O (900 mL) and stirred overnight at RT. The mixture is hydrolysed with water in THF (50%) until a precipitate is formed, which is separated off by filtration and decocted with methanol (5×100 mL). The combined organic phases are freed from the solvent using the rotary evaporator and dried (0.01 mbar/20° C.), thereby producing 9H-pyrido[2,3-b]indole-6-methanol (A3) in crystal form.

### A4) 6-benzenesulphonylmethyl-9H-pyrido[2,3-b]indole

Benzenesulphinic acid sodium salt (54.2 g, 328 mmol) is added to a suspension of 9H-pyrido[2,3-b]indol-6-methanol (A3) (13.0 g, 65.6 mmol) in 3 M HCl (100 mL) and stirred for 24 h at 80° C. The mixture is neutralised with NaHCO₃ and extracted with EtOAc: THF=1:1 (4×250 mL). The combined organic phases are washed with saturated saline solution (1×500 mL), dried (MgSO₄), filtered and freed from the solvent using the rotary evaporator. The residue is digested with iPr₂O (2×50 mL), thus producing 6-benzenesulphonylmethyl-9H-pyrido[2,3-b]indole (A4) in crystal form.

### A5) 6-(thiophene-2-sulphonylmethyl)-9H-pyrido[2,3-b]indole

6-(thiophene-2-sulphonylmethyl)-9H-pyrido[2,3-b]indole is prepared analogously to A4 from thiophene-2-sulphinic acid (Lee, C. et al., Synthesis. 1990, 5, 391-397).

### A6) 6-benzenesulphonylmethyl-9H-pyrido[2,3-b]indole-1-oxide

36% H₂O₂ (4.6 mL) is added to a suspension of 6-(thiophene-2-sulphonylmethyl)-9H-pyrido[2,3-b]indole (A5) (6 g, 18.61 mmol) in glacial acetic acid (100 mL) and the mixture is stirred for 4 h at 80° C. Then another 36% H₂O₂ (0.6 mL) are added and the mixture is stirred for a further 3 h at 80° C. The reaction solution is poured onto water (500 mL), the precipitate is filtered off and digested with water (3×150 mL), iPrOH (3×150 mL) and iPr₂O (2×150 mL), thus producing 6-benzenesulphonylmethyl-9H-pyrido[2,3-b]indole, 1-oxide (A6) in the form of a solid.

### A7) 6-(thiophene-2-sulphonylmethyl)-9H-pyrido[2,3-b]indole-1-oxide

6-(thiophene-2-sulphonylmethyl)-9H-pyrido[2,3-b]indole, 1-oxide is prepared analogously to A6 from 6-(thiophene-2-sulphonylmethyl)-9H-pyrido[2,3-b]indole (A5).

### A8) 4-chloro-6-benzenesulphonylmethyl-9H-pyrido[2,3-b]indole

Phosphorus oxychloride (7.2 mL, 77.6 mmol) is added at 10° C. to 6-benzenesulphonylmethyl-9H-pyrido[2,3-b]indol-1-oxide (A6) (3.5 g, 10.34 mmol) in anhydrous DMF (100 mL) and stirred for 1 h at 101C and 5 h at RT. The reaction mixture is poured onto water (1 L) and stirred for 20 min. The precipitate is filtered off, digested with water (4×50 mL), dissolved in the minimum amount of THF, dried (MgSO₄), filtered and freed from the solvent using the rotary evaporator. The residue is purified by column chromatography (silicon dioxide, chloroform:methanol=95:5), thus producing 4-chloro-6-benzenesulphonylmethyl-9H-pyrido[2,3-b]indole (A8) in the form of a solid.

### A9) 4-bromo-6-benzenesulphonylmethyl-9H-pyrido[2,3-b]indole

4-bromo-6-benzenesulphonylmethyl-9H-pyrido[2,3-b]indole is prepared analogously to A8.

### A10) 4-bromo-6-(thiophene-2-sulphonylmethyl)-9H-pyrido[2,3-b]indole

4-bromo-6-(thiophene-2-sulphonylmethyl)-9H-pyrido[2,3-b]indole is prepared analogously to A9 from 6-(thiophene-2-sulphonylmethyl)-9H-pyrido[2,3-b]indol-1-oxide (A7).

| # | structure | HPLC rt [min] | MS [M + H]⁺ |
|---|---|---|---|
| A4 | 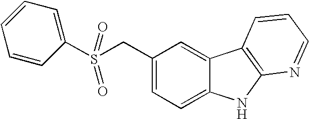 | 3.30 | 323 |
| A8 | 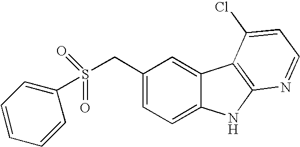 | 3.76 | 357 |
| A9 | 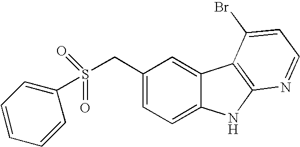 | 3.78 | 402 |
| A10 | 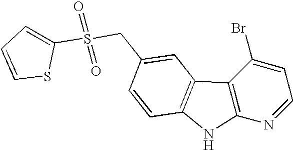 | 3.78 | 408 |

### Nucleophilic Substitution (GWM AI)

A mixture of educt (20-100 mg) and secondary amine (10 mol equivalents) are stirred in N-methylpyrrolidinone (10 μL/mg educt) in the microwave reactor for 45-60 min at 210° C. The reaction mixture is purified by preparative HPLC and the eluate is freed from the solvent by freeze-drying.

Examples 338-362 are prepared analogously to GWM AI.

| # | structure | educt | HPLC rt [min] | MS [M + H]⁺ |
|---|---|---|---|---|
| 338 | 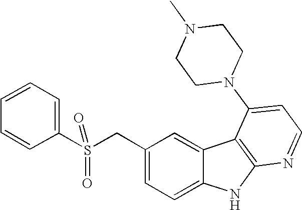 | A9 | 2.44 | 421 |
| 339 | 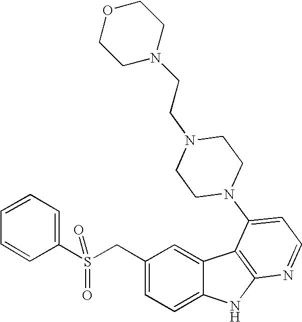 | A8 | 2.49 | 520 |
| 340 | 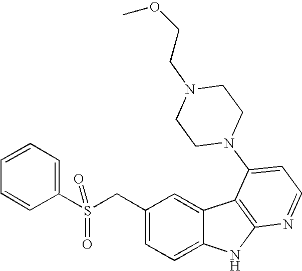 | A8 | 2.56 | 465 |
| 341 | 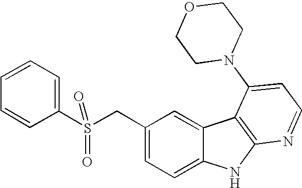 | A8 | 2.88 | 408 |
| 342 | 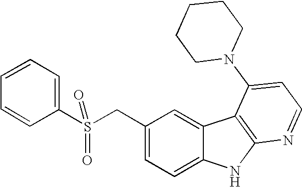 | A8 | 3.13 | 406 |
| 343 | 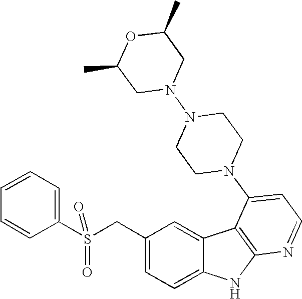 | A8 | 2.59 | 519 |
| 344 | 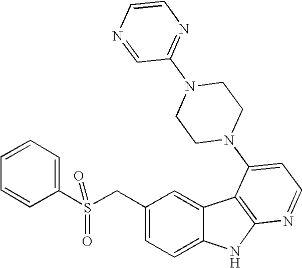 | A8 | 3.01 | 485 |
| 345 | 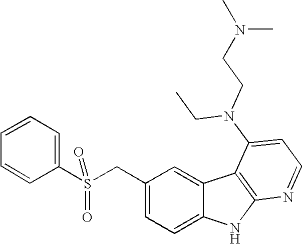 | A8 | 2.56 | 437 |
| 346 | 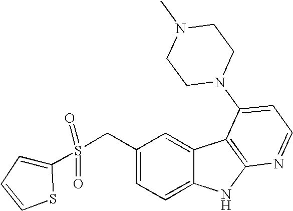 | A10 | 2.32 | 427 |
| 347 | 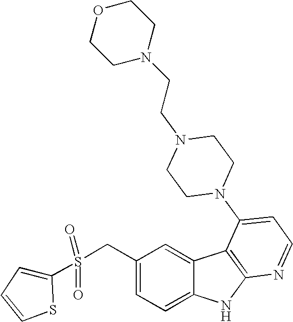 | A10 | 2.47 | 526 |
| 348 | 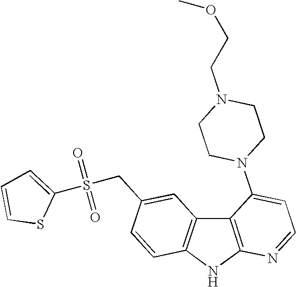 | A10 | 2.49 | 471 |
| 349 | 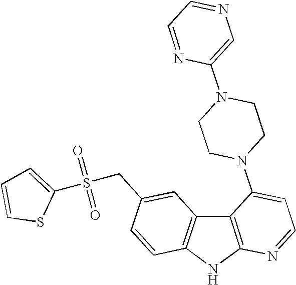 | A10 | 3.02 | 491 |
| 350 | 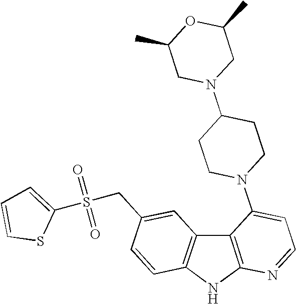 | A10 | 2.58 | 525 |
| 351 | 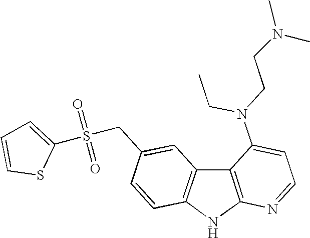 | A10 | 2.53 | 443 |
| 352 | 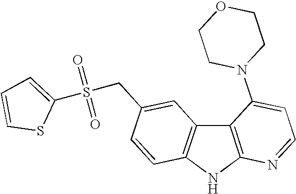 | A10 | 2.87 | 414 |
| 353 | 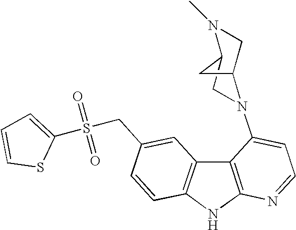 | A10 | 4.40 | 439 |
| 354 | 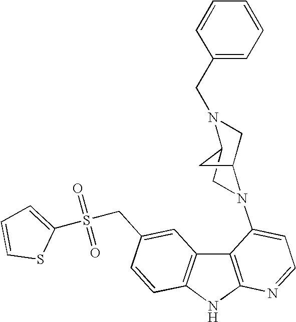 | A10 | 2.60 | 515 |
| 355 | 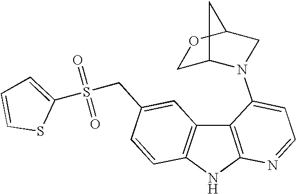 | A10 | 2.78 | 426 |
| 356 | 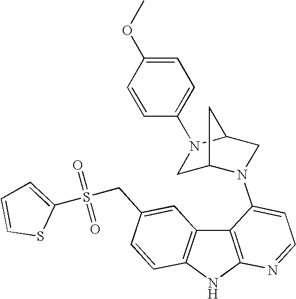 | A10 | 4.80 | 531 |
| 357 | 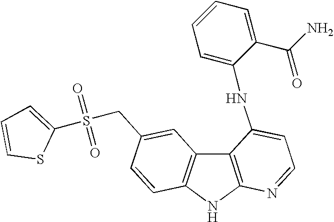 | A10 | 2.88 | 463 |
| 358 | 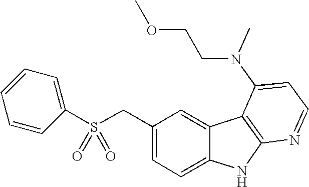 | A9 | 2.86 | 410 |
| 359 | 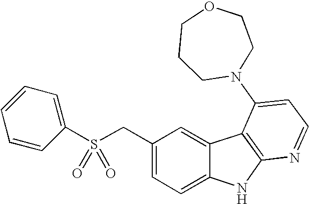 | A9 | 2.83 | 422 |
| 360 | 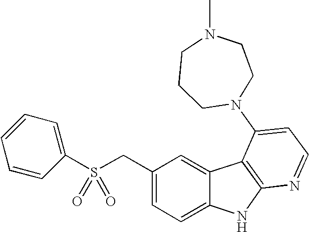 | A9 | 2.35 | 435 |
| 361 | 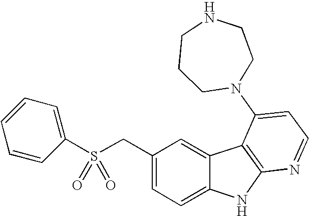 | A9 | 2.35 | 421 |
| 362 | 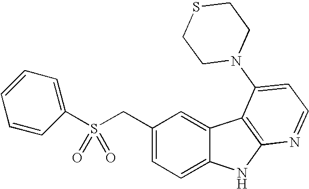 | A9 | 3.07 | 424 |

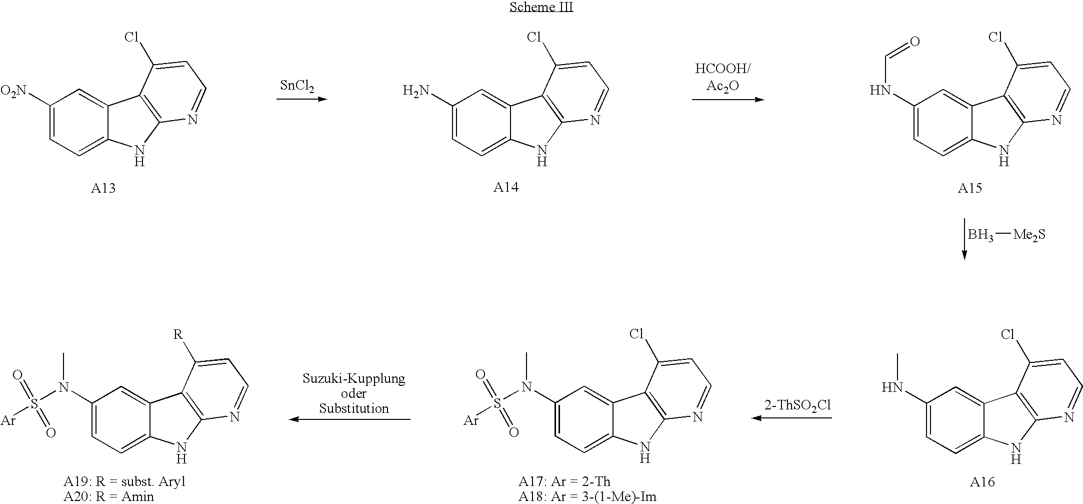

### A13) 4-chloro-6-nitro-9H-pyrido[2,3-b]indole

4-chloro-6-nitro-9H-pyrido[2,3-b]indole is prepared according to DE1913124.

### A14) 4-chloro-9H-pyrido[2,3-b]indole-6-amine

4-chloro-6-nitro-9H-pyrido[2,3-b]indole (A13) (1.4 g, 5.65 mmol) and SnCl₂*2H₂O (5.1 g, 22.6 mmol) are stirred in water (35 mL)/concentrated HCl (10 mL) for 2 h at boiling temperature and for 12 h at RT. The precipitate is filtered off and stirred in 10% NaOH (40 mL) for 30 min at RT. The precipitate is filtered off, digested with water (2×10 mL) and dried in vacuo (50° C./mbar), thereby producing 4-chloro-9H-pyrido[2,3-b]indole-6-amine (A14) as a solid.

### A15) N-(4-chloro-9H-pyrido[2,3-b]indol-6-yl)-formamide

Formic acid (5 mL) and acetic anhydride (10 mL) are stirred for 2 h at 10° C. and diluted with anhydrous THF (20 mL). 4-chloro-9H-pyrido[2,3-b]indol-6-amine (1 g, 4.59 mmol) is added batchwise over a period of 10 min and stirred for 1 h at RT. tert-Butylmethylether (50 mL) is added, the precipitate is filtered off, digested with tert-butylmethylether (2×10 mL) and dried in vacuo (50° C./mbar), thus producing N-(4-chloro-9H-pyrido[2,3-b]indol-6-yl)-formamide (A15) as a solid.

### A16) 4-chloro-N-methyl-9H-pyrido[2,3-b]indol-6-amine

Borane-dimethylsulphide complex (4.46 mL) is added dropwise at RT to N-(4-chloro-9H-pyrido[2,3-b]indol-6-yl)-formamide (A15) (4.36 g, 8.64 mmol) in anhydrous THF (40 mL) and the mixture is stirred for 2 h at RT. Then additional borane-dimethylsulphide complex (1 mL) is added dropwise and the mixture is stirred overnight at RT. Tetramethylethylenediamine (50 mL) is added and the mixture is stirred for 48 h at RT. Dilute NaHCO₃ solution (300 mL) is added, the aqueous phase is exhaustively extracted with EtOAc, and the combined organic phases are washed with NaHCO₃ (3×300 mL), water (1×300 mL) and saturated saline solution (1×300 mL), dried (MgSO₄), filtered and freed from the solvent using the rotary evaporator. The residue is dissolved in 1 N HCl (300 mL) and washed with CHCl₃ (3×50 mL). The pH of the aqueous phase is adjusted to 9 with 5 N NaOH, and the aqueous phase is exhaustively extracted with EtOAc. The combined organic phases are washed with saturated saline solution (1×200 mL), dried (MgSO₄), filtered and freed from the solvent using the rotary evaporator, thus producing 4-chloro-N-methyl-9H-pyrido[2,3-b]indol-6-amine (A16) as a solid.

### A17) N-(4-chloro-9H-pyrido[2,3-b]indol-6-yl)-N-methyl-thiophene-2-sulphonic acid amide

Pyridine (4.8 mL) is added to 4-chloro-N-methyl-9H-pyrido[2,3-b]indol-6-amine (A16) (2.1 g, 7.25 mmol) and thiophene-2-sulphonic acid chloride (1.81 g, 9.93 mmol) in anhydrous CH₂Cl₂ (150 mL) and the mixture is stirred overnight at RT. The reaction mixture is freed from the solvent using the rotary evaporator and the residue is distributed between EtOAc (100 mL) and water (50 mL). The aqueous phase is exhaustively extracted with EtOAc. The combined organic phases are washed with water (2×100 mL), 1 N NaOH (2×100 mL) and saturated saline solution (1×100 mL), dried (MgSO₄), filtered and freed from the solvent using the rotary evaporator. The residue is purified by column chromatography (SiO₂, CH₂Cl₂:methanol=95:5) and digested with Et₂O (3×5 mL), thus producing N-(4-chloro-9H-pyrido[2,3-b]indol-6-yl)-N-methyl-thiophene-2-sulphonic acid amide (A17) as a solid.

### Nucleophilic Substitution (GWM AJ)

A mixture of educt (20-100 mg) and secondary amine (10 mol equivalents) are stirred in N-methylpyrrolidinone, DMF or N,N-dimethylacetamide (10-20 μL/mg educt) in the microwave reactor for 45-60 min at 200-210° C. The reaction mixture is purified by preparative HPLC and the eluate is freed from the solvent by freeze drying or distillation using the rotary evaporator.

Examples 363-369 are prepared analogously to GWM AJ.

| # | structure | educt | HPLC rt [min] | MS [M + H]⁺ |
|---|---|---|---|---|
| 363 | 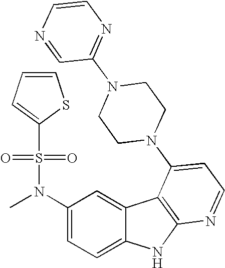 | A17 | 2.86 | 506 |
| 364 | 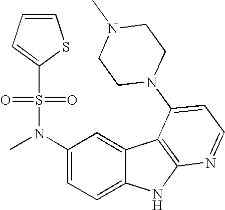 | A17 | 2.55 | 442 |
| 365 | 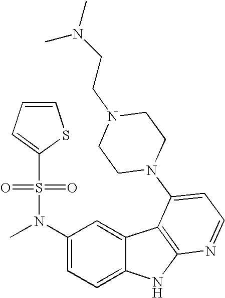 | A17 | 2.47 | 499 |
| 366 | 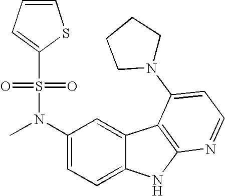 | A17 | 2.49 | 413 |
| 367 | 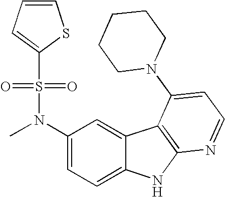 | A17 | 2.73 | 427 |
| 368 | 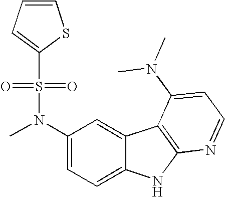 | A17 | 2.55 | 387 |
| 369 | 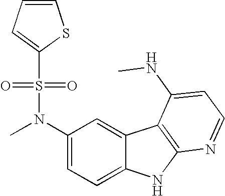 | A17 | 2.54 | 373 |

### Suzuki Coupling (GWM AK)

A mixture of educt (50-150 mg), boric acid (2 equivalents) and tetrakistriphenylphosphine palladium(0) (3-10 mol %) is stirred in ethanol/2 N aqueous Na₂CO₃ solution/toluene (in each case 400-500 μL/100 mg educt) for 900 seconds at 150° C. in the microwave reactor. The reaction mixture is diluted with water and quantitatively extracted with EtOAc. The combined organic phases are dried and evaporated down; the residue is purified by preparative HPLC and the eluate is freed from the solvent using the rotary evaporator by freeze-drying or distillation.

Examples 370-378 are prepared analogously to GWM AK.

| # | structure | educt | HPLC rt [min] | MS [M + H]⁺ |
|---|---|---|---|---|
| 370 | 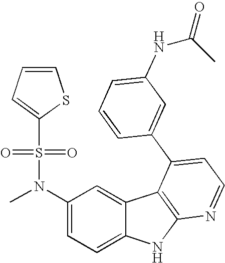 | A17 | 3.02 | 477 |
| 371 | 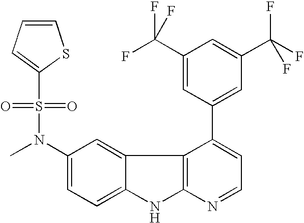 | A17 | 3.62 | 556 |
| 372 | 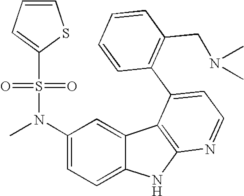 | A17 | 2.60 | 477 |
| 373 | 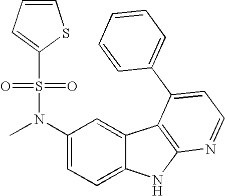 | A17 | 3.31 | 420 |
| 374 | 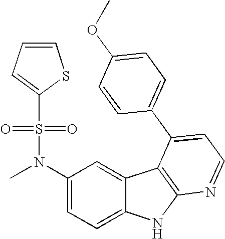 | A17 | 3.25 | 450 |
| 375 | 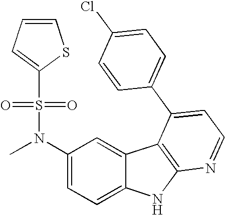 | A17 | 3.49 | 454 |
| 376 | 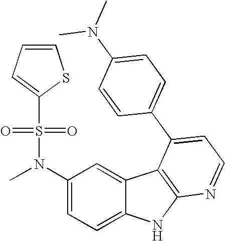 | A17 | 3.26 | 463 |
| 377 | 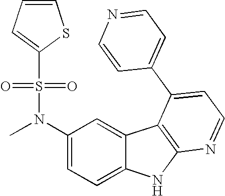 | A17 | 2.73 | 421 |
| 378 | 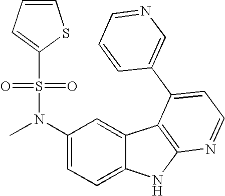 | A17 | 2.84 | 421 |

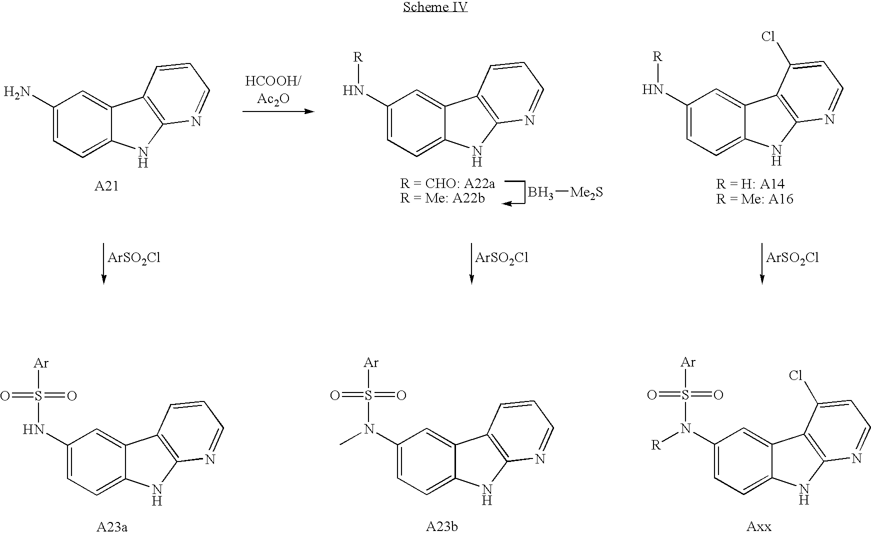

### A21) 9H-pyrido[2,3-b]indol-6-ylamine

9H-pyrido[2,3-b]indol-6-ylamine (A21) is prepared according to Stephenson, L et al.; J. Chem. Soc. C, 1970, 10, 1355-1364.

### A22a) N-(9H-pyrido[2,3-b]indol-6-yl)-formamide

Formic acid (1.34 mL) and acetic anhydride (3 mL) are stirred for 1 h at 60° C. and then diluted with anhydrous dioxane (40 mL). 9H-pyrido[2,3-b]indol-6-ylamine (A21) (2 g, 10.91 mmol) is added batchwise over a period of 10 min at 10° C. and stirred overnight at RT. The reaction mixture is freed from the solvent using the rotary evaporator and the residue is digested with water (4×25 mL), iPrOH (2×25 mL) and tert-butylmethylether (3×25 mL), dissolved in formic acid (5 mL) and distributed between 0.1 N HCl (100 mL) and water (100 mL). The organic phase is exhaustively extracted with 0.1 N HCl, and the combined aqueous phases are washed with EtOAc (5×100 mL). The pH value of the aqueous phase is adjusted to 9 with 5 N NaOH, the precipitate is isolated by filtration and dried (50° C., 1 mbar), thereby yielding N-(9H-pyrido[2,3-b]indol-6-yl)formamide (A22a) as a solid.

### A22b) N-methyl-9H-pyrido[2,3-b]indol-6-amine

Lithium aluminium hydride (3.5 M in Et₂O, 2 mL, 7 mmol) is added dropwise to a suspension of N-(9H-pyrido[2,3-b]indol-6-yl)-formamide (A22a) (450 mg, 2.13 mmol) in anhydrous Et₂O (200 mL) within 5 min at RT and stirred for 5 h at this temperature. THF (50 mL), water (40 mL) and 5 N NaOH (20 mL) are added, and the aqueous phase is exhaustively extracted with EtOAc. The combined organic phases are washed with saturated saline solution (1×100 mL), dried (MgSO₄), filtered and freed from the solvent using the rotary evaporator. The residue is digested with iPr₂O (2×50 mL), thereby yielding N-methyl-9H-pyrido[2,3-b]indol-6-amine (A22b) in crystal form.

### Sulphonic Acid Amide Formation (GWM AL)

Pyridine (6 equivalents) is added to a mixture of the corresponding amine (A 14, A16, A21 or A22b, 50-200 mg) and arylsulphonic acid chloride (1.1 to 2 equivalents) in anhydrous CH₂Cl₂ (5 mL/100 mg amine) and stirred overnight at RT. The reaction mixture is freed from the solvent using the rotary evaporator, the residue is purified by preparative HPLC and the eluate is freed from the solvent using the rotary evaporator by freeze-drying or distillation.

Examples 379-390 are prepared analogously to GWM AL.

| | | HPLC | MS |
|---|---|---|---|
| # | structure | rt [min] | [M + H]⁺ |
| 379 | 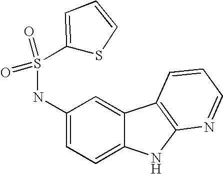 | 2.80 | 330 |
| 380 | 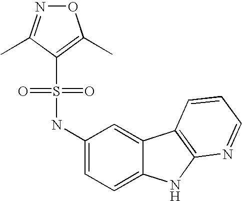 | 2.84 | 343 |
| 381 | 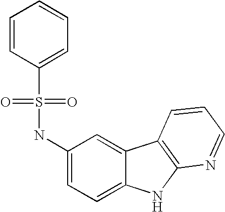 | 2.82 | 324 |
| 382 | 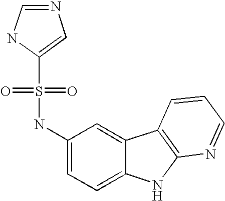 | 0.36 | 314 |
| 383 | 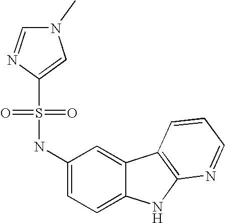 | 0.36 | 328 |
| 384 | 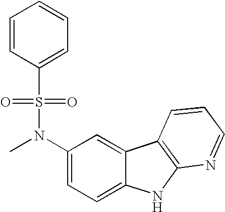 | 2.98 | 338 |
| 385 | 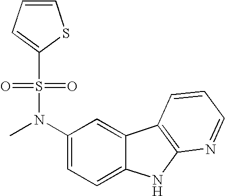 | 2.94 | 344 |
| 386 | 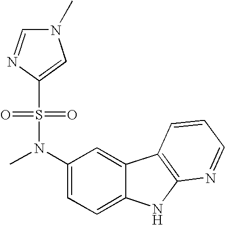 | 2.42 | 342 |
| 387 | 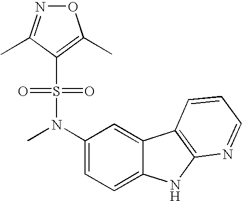 | 2.96 | 357 |
| 388 | 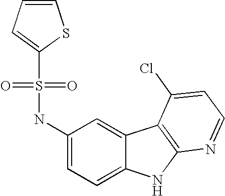 | 3.07 | 364 |
| 389 | 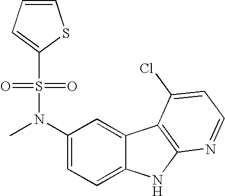 | 3.21 | 378 |
| 390 | 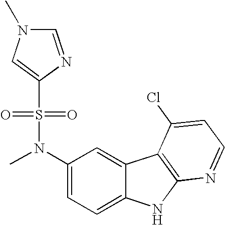 | 2.76 | 376 |

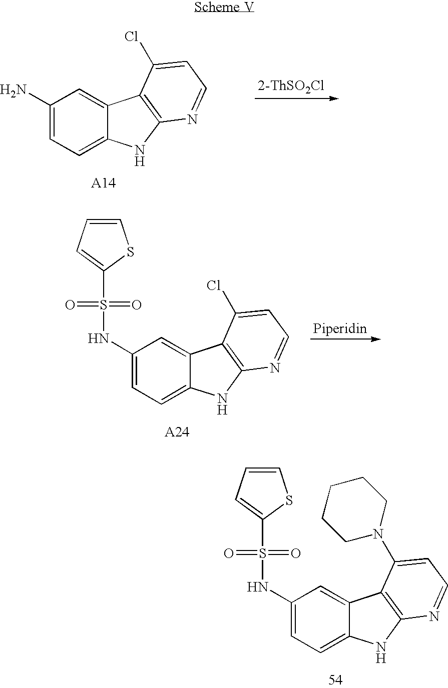

### A24) (4-chloro-9H-pyrido[2,3-b]indol-6-yl)-thiophene-2-sulphonic acid amide

Pyridine (145 μL) is added to 4-chloro-9H-pyrido[2,3-h]indol-6-amine (A14) (65 mg, 0.3 mmol) and thiophene-2-sulphonic acid chloride (62 mg, 0.33 mmol) in anhydrous CH₂Cl₂ (2 mL) and the mixture is stirred for 3 h at RT. The reaction mixture is freed from the solvent using the rotary evaporator and purified by preparative HPLC. After concentration by evaporation of the corresponding fractions (4-chloro-9H-pyrido[2,3-h]indol-6-yl)-thiophene-2-sulphonic acid amide (A24) is obtained as a foam.

### EXAMPLE 391

(4-chloro-9H-pyrido[2,3-h]indol-6-yl)-thiophene-2-sulphonic acid amide (A24) (50 mg, 0.137 mmol), piperidine (52 μL) and DMF (800 μL) are stirred in the microwave reactor for 25 min at 200° C. g. The reaction mixture is freed from the solvent using the rotary evaporator and is purified by preparative HPLC. After concentration by evaporation of the corresponding fractions 4-(piperidin-1-yl)-9H-pyrido[2,3-b]indol-6-yl)thiophene-2-sulphonic acid amide is obtained as a foam.

| # | structure | HPLC rt [min] | MS [M + H]⁺ |
|---|---|---|---|
| 391 | 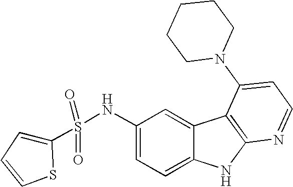 | 2.81 | 413 |

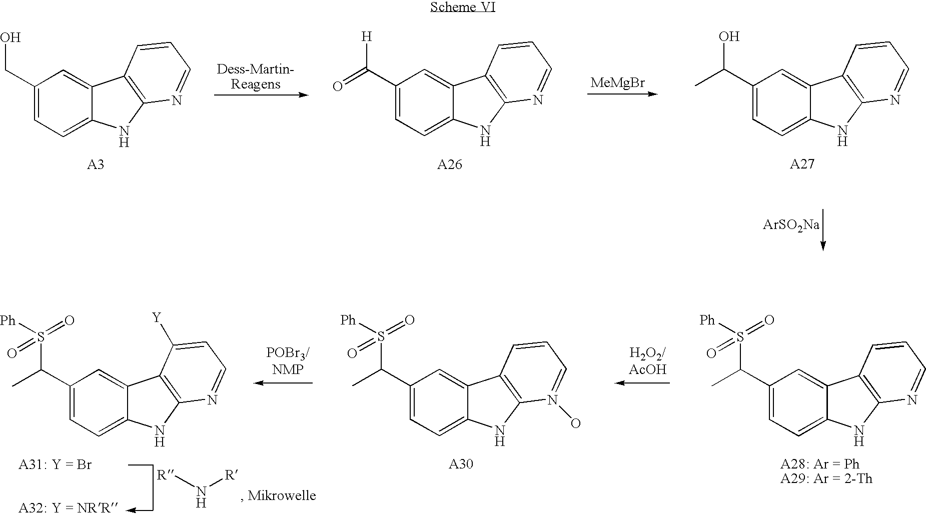

### A26) 9H-pyrido[2,3-b]indole-6-carbaldehyde

Dess-Martin Periodinane (15.1 g, 35.4 mmol) in Anhydrous CH₂Cl₂

(60 mL) is added at RT over a period of 2 min to 9H-pyrido[2,3-b]indole-6-methanol (A3) (4.4 g, 22.2 mmol) in anhydrous CH₂Cl₂ (60 mL) and the mixture is stirred for 2.5 h. The same amount of periodinane is metered in and the mixture is stirred for another 30 min. It is diluted with CH₂Cl₂ (200 mL) and washed with semisaturated NaHCO₃ solution to which sodium thiosulphate has been added. The aqueous phase is exhaustively extracted with CH₂Cl₂. The combined organic phases are washed with semisaturated NaHCO₃ solution (2×300 mL) and saturated saline solution (1×100 mL), dried (MgSO₄), filtered and freed from the solvent using the rotary evaporator. The residue is digested with iPr₂O (2×20 mL), thereby yielding 9H-pyrido[2,3-b]indole-6-carbaldehyde (A26) in the form of crystals.

### A27) 1-(9H-pyrido[2,3-b]indol-6-yl)ethanol

Methylmagnesium bromide (3 M in ether, 15 mL, 45 mmol) is added at 0° C. to a solution of 9H-pyrido[2,3-b]indole-6-carbaldehyde (A26) (2.2 g, 11.2 mmol) in anhydrous THF (220 mL) and stirred for 2 h at RT. Saturated ammonium chloride solution (150 mL) is added and the aqueous phase is quantitatively extracted with EtOAc. The combined organic phases are washed with water (2×300 mL) and saturated saline solution (1×100 mL), dried (MgSO₄), filtered and freed from the solvent using the rotary evaporator, thereby yielding 1-(9H-pyrido[2,3-b]indol-6-yl)ethanol (A27) in the form of crystals.

### A28) 6-(1-benzenesulphonylethyl)-9H-pyrido[2,3-b]indole

1-(9H-pyrido[2,3-b]indol-6-yl)ethanol (A27) (1 g, 4.71 mmol) and benzenesulphinic acid sodium salt (3.09 g, 18.8 mmol) are stirred in formic acid (40 mL) for 2 h at 95° C. The solvent is eliminated using the rotary evaporator, the residue is distributed between water (500 mL) and EtOAc (500 mL) and the aqueous phase is quantitatively extracted with EtOAc. The combined organic phases are washed with saturated potassium carbonate solution (2×500 mL) and saturated saline solution (1×500 mL), dried (MgSO₄), filtered and freed from the solvent using the rotary evaporator. The residue is crystallised under EtOAc, thereby yielding 6-(1-benzenesulphonyl-ethyl)-9H-pyrido[2,3-b]indole (A28) in the form of crystals.

### A29) 6-[1-(thiophene-2-sulphonyl)ethyl]-9H-pyrido[2,3-b]indole

6-[1-(thiophene-2-sulphonyl)-ethyl]-9H-pyrido[2,3-b]indole (A29) is prepared analogously to 6-(1-benzenesulphonylethyl)-9H-pyrido[2,3-b]indole (A28) from thiophenesulphinic acid sodium salt (Crowell et al., J. Med. Chem. 1989, 32, 2436-2442).

### A30) 6-(1-benzenesulphonylethyl)-9H-pyrido[2,3-b]indole-1-oxide

6-(1-benzenesulphonylethyl)-9H-pyrido[2,3-b]indole (A28) (1 g, 2.97 mmol) and 30% H₂O₂ (2.5 mL) are stirred in acetic acid (10 mL) for 12 h at 80° C. The mixture is distributed between water (200 mL) and EtOAc (200 mL) and the aqueous phase is quantitatively extracted with EtOAc. The combined organic phases are washed with water (5×150 mL), saturated sodium thiosulphate solution (2×100 mL), saturated potassium carbonate solution (2×100 mL) and saturated saline solution (1×100 mL), dried (MgSO₄), filtered and freed from the solvent using the rotary evaporator, thereby yielding 6-(1-benzenesulphonylethyl)-9H-pyrido[2,3-b]indole-1-oxide (A30) in the form of crystals.

### A31) 6-(1-benzenesulphonylethyl)-4-bromo-9H-pyrido[2,3-b]indole

6-(1-benzenesulphonylethyl)-9H-pyrido[2,3-b]indole-1-oxide (A30) (200 mg, 0.31 mmol) and phosphorus oxybromide (325 mg, 1.13 mmol) are stirred in anhydrous N-methylpyrrolidinone (3 mL) 1 h at RT. The mixture is distributed between water (50 mL) and EtOAc (50 mL) and the aqueous phase is quantitatively extracted with EtOAc. The combined organic phases are washed with water (3×50 mL) and saturated saline solution (1×50 mL), dried (MgSO₄), filtered and freed from the solvent using the rotary evaporator, thereby yielding 6-(1-benzenesulphonylethyl)-4-bromo-9H-pyrido[2,3-b]indole (A31) in the form of a foam.

### EXAMPLE 392

6-(1-benzenesulphonylethyl)-4-bromo-9H-pyrido[2,3-b]indole (A31) (30 mg, 0.07 mmol) and N-methylpiperazine (300 μL) are stirred in the microwave reactor for 80 min at 170° C. and evaporated down using the rotary evaporator. The crude product is purified by column chromatography (neutral aluminium oxide, CH₂Cl₂:methanol=20:1), thereby yielding 6-(1-benzenesulphonylethyl)-4-(4-methylpiperazin-1-yl)-9H-pyrido[2,3-b]indole as an oil.

| # | structure | HPLC rt [min] | MS [M + H]⁺ |
|---|---|---|---|
| 392 | 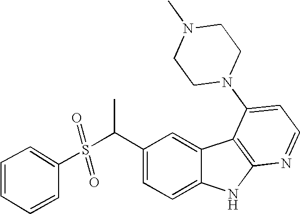 | 2.42 | 413 |

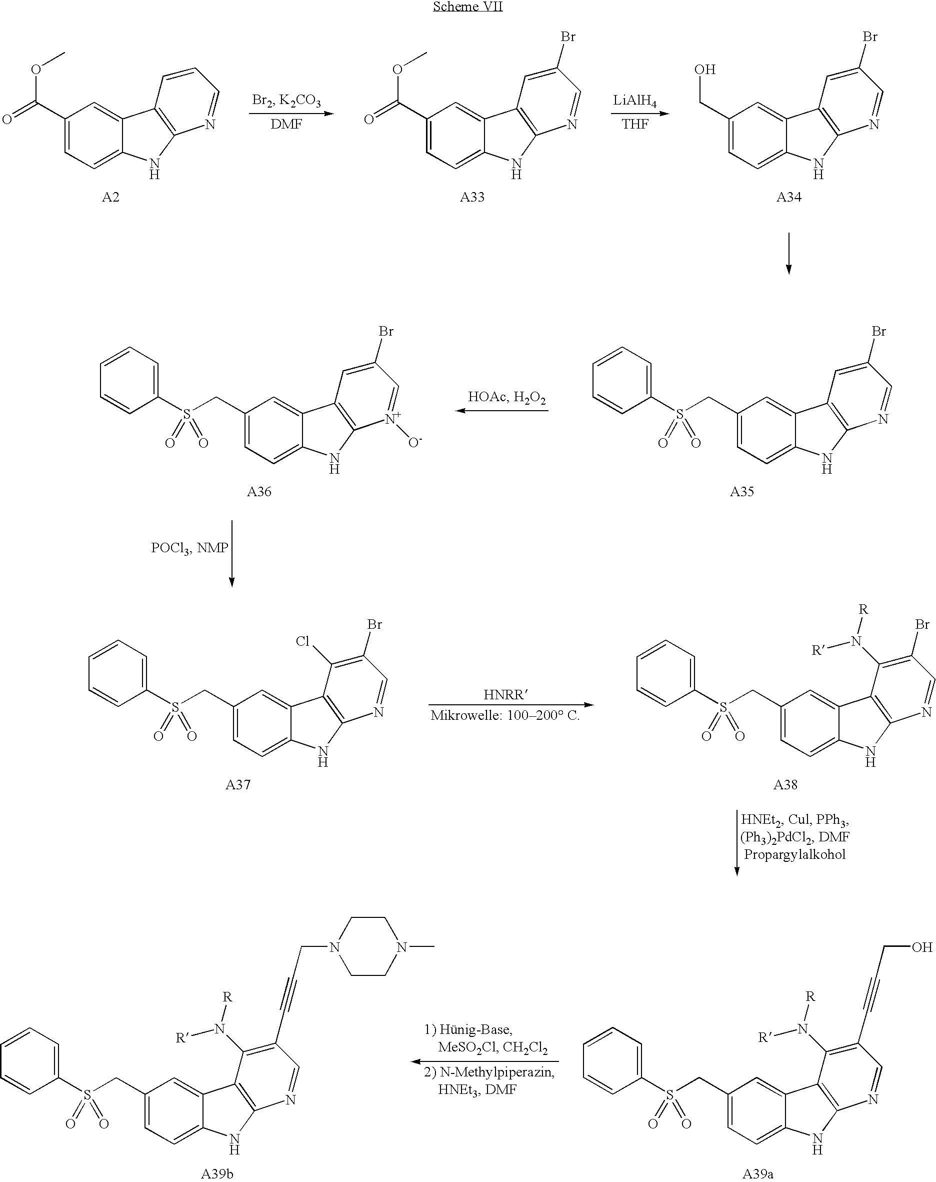

### A33) methyl 3-bromo-9H-pyrido[2,3-b]indole-6-carboxylate

A solution of bromine (1.18 ml, 22.89 mmol) in 10 mL DMF is slowly added dropwise to a suspension of methyl 9H-pyrido[2,3-b]indole-6-carboxylate (A2) (5.13 g, 22.67 mmol) and potassium carbonate (3.16 g, 22.89 mmol) at −60° C. under an argon atmosphere and the mixture is stirred overnight in the cooling bath, while the temperature rises to RT. For working up the suspension is combined with 10 mL DMF, the precipitate is filtered off, digested with ethyl acetate, filtered off and the filtrate is combined with water. The precipitate is filtered off, washed with water and dried in vacuo. Methyl 3-bromo-9H-pyrido[2,3-b]indole-6-carboxylate (A33) is obtained in the form of crystals.

### A34) (3-bromo-9H-pyrido[2,3-b]indol-6-yl)-methanol

Lithium aluminium hydride (1.37 g, 34.92 mmol) is added batchwise under an argon atmosphere to a suspension of methyl 3-bromo-9H-pyrido[2,3-b]indole-6-carboxylate (A33) (7.35 g, 24.08 mmol) in 100 mL THF. Then the mixture is stirred for 1.5 h at RT. For working up, potassium sodium tartrate solution is added while cooling with ice and the mixture is stirred until no more gas is given off. It is combined with sodium sulphate (anhydrous), briefly stirred, filtered off through Celite and washed with a little EtOAc. Evaporating the filtrate to dryness, digesting with 50 mL EtOAc, filtering through Celite and further evaporation in vacuo yields (3-bromo-9H-pyrido[2,3-b]indol-6-yl)-methanol (A34) in the form of crystals.

### A35) 6-benzenesulphonylmethyl-3-bromo-9H-pyrido[2,3-b]indole

A solution of (3-bromo-9H-pyrido[2,3-b]indol-6-yl)-methanol (A34) (5.48 g, 19.78 mmol) and benzenesulphinic acid sodium salt (16.35 g, 99.62 mmol) in 60 mL formic acid is heated to 90° C. for 3 h. It is cooled to RT and taken up in twice the volume of EtOAc and washed 5 times with saturated NaHCO₃ solution. The organic phase is separated off and dried on sodium sulphate (anhydrous) and evaporated down in vacuo. Digesting the crude product with 100 mL toluene, filtering off the crystals and drying under high vacuum yields 6-benzenesulphonylmethyl-3-bromo-9H-pyrido[2,3-b]indole.

### A36) 6-benzenesulphonylmethyl-3-bromo-9H-pyrido[2,3-b]indole 1-oxide

A solution of 6-benzenesulphonylmethyl-3-bromo-9H-pyrido[2,3-b]indole (A35) (5.64 g, 14.06 mmol) in 240 mL acetic acid is combined with 45 mL 30% aqueous H₂O₂ solution and the mixture is stirred for 12 h at 80° C. The reaction mixture is combined with water, the precipitate formed is filtered off and dried under high vacuum. 6-Benzenesulphonyl-methyl-3-bromo-9H-pyrido[2,3-b]indole 1-oxide (A36) is obtained as a solid.

### A37) 6-benzenesulphonylmethyl-3-bromo-4-chloro-9H-pyrido[2,3-b]indole

Phosphorus oxychloride (POCl₃) (3.3 mL, 36 mmol) is added batchwise under an argon atmosphere at −20° C. to a suspension of 6-benzenesulphonylmethyl-3-bromo-9H-pyrido[2,3-b]indole-1-oxide (A36) (3 g, 7.20 mmol) in 40 mL N-methylpyrrolidone and the mixture is allowed to thaw to RT within 2 h with stirring. Then while cooling with ice it is combined with twice the volume of water and the mixture is stirred for 15 min in the ice bath. The precipitate formed is filtered off, washed with water and dried in a high vacuum. 6-Bbenzenesulphonylmethyl-3-bromo-4-chloro-9H-pyrido[2,3-b]indole (A37) is obtained in the form of crystals.

| # | Structure | HPLC rt [min] | MS [M + H]⁺ |
|---|---|---|---|
| A33 | 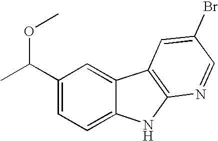 | 3.86 | 305 |
| A35 | 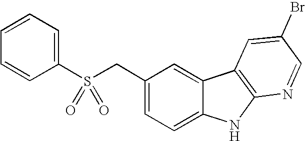 | 3.82 | 401 |
| A36 | 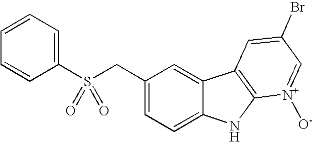 | 1.64 | 417 |
| A37 | 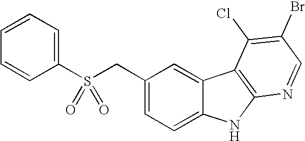 | 4.04 | 435 |

### Nucleophilic Substitution (GWM AM)

A mixture of 6-benzenesulphonylmethyl-3-bromo-4-chloro-9H-pyrido[2,3-b]indole (A37) (20-100 mg) and secondary amine (10 mol equivalents) is stirred in N-methylpyrrolidinone, DMF or N,N-dimethylacetamide (10-20 μL/1 mg educt) in the microwave reactor for 20-40 min at 180-210° C. The reaction mixture is purified by preparative HPLC and the eluate is freed from the solvent using the rotary evaporator by freeze-drying or distillation.

### EXAMPLE 393

A solution of 6-benzenesulphonylmethyl-3-bromo-4-morpholin-4-yl-9H-pyrido[2,3-b]indole (56) (0.1 g, 0.21 mmol), propargylalcohol (0.03 mL, 0.51 mmol), diethylamine (0.32 mL, 3.08 mmol), CuI (2.2 mg, 0.01 mmol), triphenylphosphine (10.8 mg, 0.04 mmol) and bis [diphenyl-[4-(1H, 1H,2H,2H-perfluorodecyl)phenyl]phosphine]palladium (II) chloride [(PPH₃)₂PdCl₂] (8.2 mg, 0.01 mmol) in 0.5 mL anhydrous DMF is heated to 120° C. for 30 min under argon in the microwave reactor. It is taken up in 60 mL of EtOAc and extracted twice with saturated aqueous ammonium chloride solution. The organic phase is dried on sodium sulphate (anhydrous), the crude product is taken up in 1.5 mL DMF and purified by preparative HPLC. The eluate is freed from the solvent by freeze-drying. 3-(6-Benzenesulphonylmethyl-4-morpholin-4-yl-9H-pyrido[2,3-b]indol-3-yl)-prop-2-yn-1-ol is obtained in the form of crystals.

### EXAMPLE 394

To a suspension of 3-(6-benzenesulphonylmethyl-4-morpholin-4-yl-9H-pyrido[2,3-b]indol-3-yl)-prop-2-yn-1-ol (56) (14 mg, 0.03 mmol) in 2 mL anhydrous dichloromethane are added successively, under argon, diisopropylamine (0.01 mL, 0.1 mmol) and methanesulphonyl chloride (3.6 μL, 0.05 mmol) and the mixture is stirred for 3 h at RT. The solvent is eliminated in vacuo without heating and the residue is taken up in 2 mL anhydrous DMF, combined with N-methylpiperazine (0.05 mL, 0.45 mmol) and triethylamine (0.1 mL) and stirred for 2 h at RT. The reaction mixture is evaporated to dryness in vacuo, taken up in DMF and purified by preparative HPLC. The eluate is freed from the solvent by freeze-drying. 6-Benzenesulphonylmethyl-3-[3-(4-methyl-piperazin-1-yl)-prop-1-ynyl]-4-morpholin-4-yl-9H-pyrido[2,3-b]indole is obtained as a solid.

### EXAMPLES 393-398

| # | structure | HPLC rt [min] | MS [M + H]⁺ |
|---|---|---|---|
| 393 | 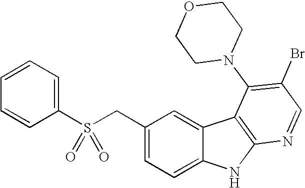 | 3.93 | 486 |
| 394 | 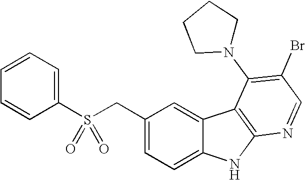 | 4.38 | 470 |
| 395 | 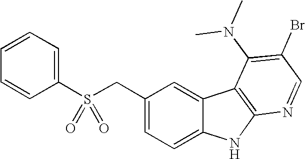 | 4.18 | 444 |
| 396 | 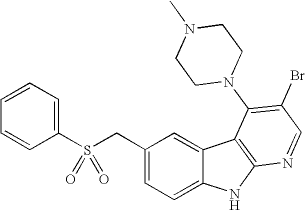 | 2.77 | 499 |
| 397 | 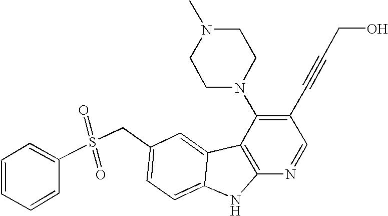 | 3.34 | 462 |
| 398 | 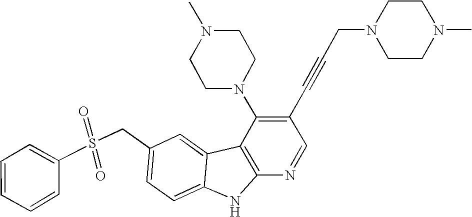 | 2.94 | 544 |

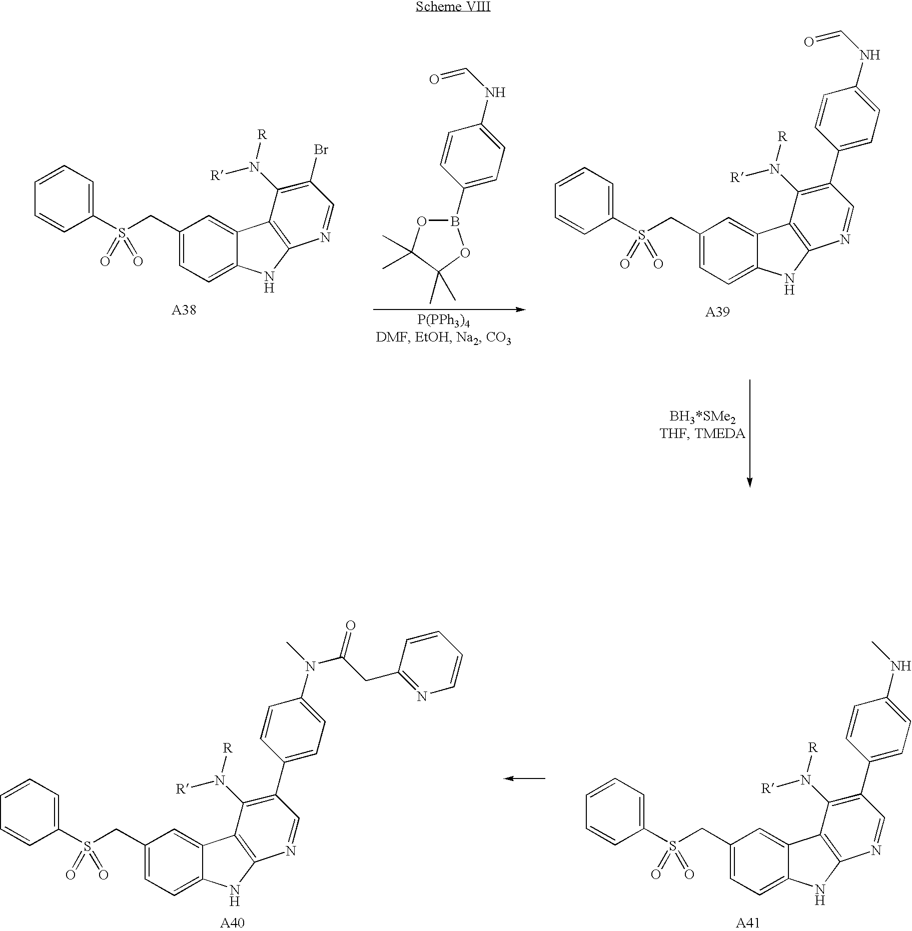

### EXAMPLE 399

A suspension of 6-benzenesulphonylmethyl-3-bromo-4-(4-methyl-piperazin-1-yl)-9H-pyrido[2,3-b]indole (58) (0.1 g, 0.2 mmol), N-(4-(4,4,5,5-tetramethyl-1,3,2-dioxaborolan-2-yl)-phenyl)-formamide, P(PH₃)₄ (23 mg, 0.02 mmol) in 1 mL each of DMF/ethanol/saturated Na₂CO₃ solution is stirred for 15 min at 120° C. under an argon atmosphere in the microwave reactor. The mixture is combined with EtOAc, extracted twice with saturated Na₂CO₃ solution and once with water. The combined organic phases are dried on anhydrous sodium sulphate and the solvent is evaporated down in vacuo. The reaction mixture is taken up in DMF and purified by preparative HPLC. Freeze-drying the eluate yields N-{4-[6-benzenesulphonyl-methyl-4-(4-methyl-piperazin-1-yl)-9H-pyrido[2,3-b]indol-3-yl]-phenyl}-formamide.

| # | structure | HPLC rt [min] | MS [M + H]⁺ |
|---|---|---|---|
| 399 | 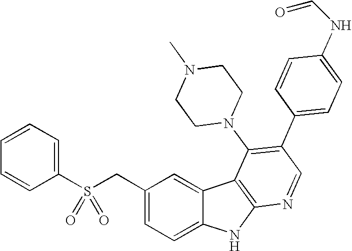 | 2.77 | 540 |

### Reduction to N-methylcarbolinamines (GWM AN)

Borane-dimethylsulphide complex or borane-THF complex (2-20 equivalents) is added dropwise at RT to a solution of the starting compound in anhydrous THF (10-50 mL) and the mixture is stirred for 2-10 h at RT. Then additional borane complex is optionally added dropwise and the mixture is stirred overnight at RT. Tetramethylethylenediamine (10-50 equivalents) is added and the mixture is stirred for 48 h at RT. Dilute NaHCO₃ solution is added, the aqueous phase is exhaustively extracted with EtOAc, and the combined organic phases are washed with NaHCO₃, water and saturated saline solution, dried (MgSO₄), filtered and freed from the solvent using the rotary evaporator. The product thus obtained is used directly for further reaction without being purified.

### EXAMPLE 400

| # | structure |
|---|---|
| 400 | 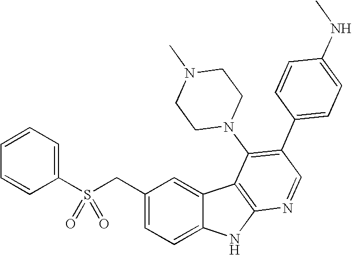 |

### Formation of Carboxamides (GWM AO)

Method 1 Starting from Acid Chlorides or Anhydrides

The acid chloride or the anhydride (1.1-5 equivalents), in substance or as a solution in anhydrous CH₂Cl₂, and then a base (triethylamine, pyridine, N-ethyldiisopropylamine or potassium carbonate; 3-50 equivalents) are added successively to a solution of the amine in anhydrous CH₂Cl₂ (10-100 mL/1 g educt) and stirred for 1-12 h at RT. The reaction solution is diluted with CH₂Cl₂, washed with water, saturated ammonium chloride solution, saturated NaHCO₃ solution and saturated saline solution, dried (Na₂SO₄), filtered, freed from the solvent using the rotary evaporator and the crude product is optionally purified by chromatography.

Method 2 Starting from Carboxylic Acids Using TBTU

A solution of amine, carboxylic acid (1 equivalent), TBTU (1.2 equivalents) and a base (triethylamine, N-ethyldiisopropylamine, or pyridine; 1-5 equivalents) in anhydrous DMF (10-20 mL/1 g amine) are stirred for 2-24 h at RT. If necessary further carboxylic acid and TBTU are metered in. The reaction solution is freed from the solvent using the rotary evaporator, the residue is taken up in CH₂Cl₂, washed with water, saturated ammonium chloride solution, saturated NaHCO₃ solution and saturated saline solution, dried (Na₂SO₄), filtered, freed from the solvent using the rotary evaporator and the crude product is optionally purified by chromatography.

### EXAMPLE 401

| | | HPLC rt | MS |
|---|---|---|---|
| # | structure | [min] | [M + H]⁺ |
| 401 | 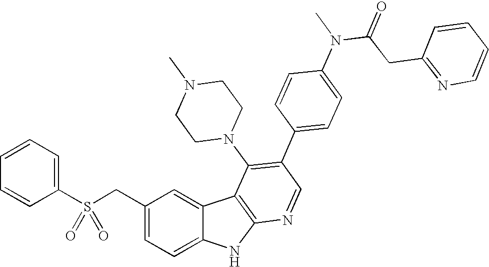 | 2.86 | 645 |

Biological Properties

As demonstrated by DNA staining followed by FACS analysis, the inhibition of proliferation brought about by the compounds according to the invention is mediated above all by the arrest of the cells in the G2/M phase of the cell cycle. The cells arrest, depending on the type of cell used, for a specific length of time in this cell cycle phase before programmed cell death is initiated. An arrest in the G2/M phase of the cell cycle may be initiated e.g. by the inhibition of specific cell cycle kinases. On the basis of their biological properties the compounds of general formula (1) according to the invention, their isomers or the physiologically acceptable salts thereof are suitable for treating diseases characterised by excessive or anomalous cell proliferation.

Inhibition of Cyclin/CDK Enzyme Activity In Vitro

High Five™ insect cells (Trichoplusia ni) which have been infected with a high titre of recombinant baculovirus are used to produce active human cyclin/CDK holoenzymes. cDNA for cyclin B1 or CDK1 is expressed in the baculovirus expression system. Cyclin B1 is used as a fusion protein with GST, whereas CDK1 is expressed without a tag. Insect cells are co-infected with baculoviruses for CycB1-GST and CDK1 and incubated for 3 days to achieve optimum expression of the complex.

To prepare the active holoenzyme, cells are lysed and the soluble total protein fraction is separated off by centrifugation of cell residues and insoluble components. This total cell lysate is used as a protein source for kinase tests.

The substrate Histone H1 (Sigma) is used for the kinase assay. Lysates of the insect cells infected with recombinant baculovirus are incubated together with ATP (final concentration 8 μM), radiolabelled ³³P-ATP in the presence of the substrate with various concentrations of the inhibitor (12 concentrations, beginning at 166 μM or 16 μM) for 50 min at 30° C. The reaction is stopped with 5% TCA (trichloroacetic acid) and cooled for 30 min. The substrate proteins with associated radioactivity are transferred onto GFB filter plates (Perkin Elmer), washed 4 times with water, dried and after the addition of scintillation cocktail measured in a Wallace 1450 Microbeta Liquid Scintillation Counter. For each concentration of the substance double measurements are carried out; IC₅₀ values are calculated with GraphPad Prizm.

Inhibition of the Proliferation of Cultivated Human Tumour Cells

Cells of the non-small cell lung tumour cell line NCI-H460 (American Type Culture Collection (ATCC HTB 177)) are cultivated in Iscove's Modified Dulbecco Medium IMDM (Bio Whittaker), supplemented with 25 nM Hepes, L-glutamine (2 mmol), 100 U/mL penicillin/100 μg/mL streptomycin and 10% foetal calf serum (Gibco) and harvested in the logarithmic growth phase. Then the NCI-H460 cells are seeded in 96 multi-well flat-bottomed dishes (Nunc) at a density of 2500 cells per well in 190 μL medium and incubated overnight in an incubator. Different concentrations of the compounds (dissolved in DMSO; final concentration: <1%) are added to the cells in a volume of 10 μL. Seven different dilutions (from 5.5 μM downwards in steps of three) are tested. Control wells have no test compounds added to them. If necessary (depending on the potency of the substances) the concentration range tested is adjusted. After 72 h incubation ³H-thymidine (Amersham) is added to each well and incubation is continued for a further 16 h. The amount of ³H-thymidine which is incorporated into the tumour cells in the presence of the inhibitor and which represents the number of cells in the S phase, is measured in a Wallace 1450 Microbeta Liquid Scintillation Counter. IC₅₀ values for the inhibition of the proliferation (=inhibition of incorporated ³H-thymidine) are calculated—correcting for the background radiation—and analysed with GraphPad Prizm. All the measurements are done three times.

All the compounds shown have an IC₅₀ value below 500 nM in the test.

Arresting the Tumour Cells in the G2/M Phase of the Cell Cycle

1.7 5×10⁶ cells (non-small cell lung tumour NCI-H460) are seeded in T75 cell culture flasks. After 24 h test substance is added and incubation is continued for a further 24 h. Then the supernatant is collected, the cells are detached with trypsin, combined with the supernatant and centrifuged. The cell pellet is washed with buffered saline solution (PBS) and the cells are then fixed with 80% ethanol at −20° C. for at least 2 h. After another washing step with PBS the cells are permeabilised with Triton-X100 (Sigma; 0.25% in PBS) for 5 min on ice and then incubated with a solution of propidium iodide (Sigma; 10 g/ml) and RNAse (Serva; 1 mg/mL) in the ratio 9:1.

All the compounds shown have an EC₅₀ value below 1000 nM in the test.

The substances of the present invention are serine-threonine kinase inhibitors. On the basis of their biological properties the new compounds of general formula (1), their isomers and the physiologically acceptable salts thereof are suitable for treating diseases characterised by excessive or anomalous cell proliferation.

Such diseases include for example: viral infections (e.g. HIV and Kaposi's sarcoma); inflammatory and autoimmune diseases (e.g. colitis, arthritis, Alzheimer's disease, glomerulonephritis and wound healing); bacterial, fungal and/or parasitic infections; leukaemias, lymphomas and solid tumours; skin diseases (e.g. psoriasis); bone diseases; cardiovascular diseases (e.g. restenosis and hypertrophy). They are also useful for protecting proliferating cells (e.g. hair, intestinal, blood and progenitor cells) from DNA damage caused by radiation, UV treatment and/or cytostatic treatment (Davis et al., 2001).

For example, the following cancers may be treated with compounds according to the invention, without being restricted thereto: brain tumours such as for example acoustic neurinoma, astrocytomas such as pilocytic astrocytomas, fibrillary astrocytoma, protoplasmic astrocytoma, gemistocytary astrocytoma, anaplastic astrocytoma and glioblastoma, brain lymphomas, brain metastases, hypophyseal tumour such as prolactinoma, HGH (human growth hormone) producing tumour and ACTH producing tumour (adrenocorticotropic hormone), craniopharyngiomas, medulloblastomas, meningeomas and oligodendrogliomas; nerve tumours (neoplasms) such as for example tumours of the vegetative nervous system such as neuroblastoma sympathicum, ganglioneuroma, paraganglioma (pheochromocytoma, chromaffinoma) and glomus-caroticum tumour, tumours on the peripheral nervous system such as amputation neuroma, neurofibroma, neurinoma (neurilemmoma, Schwannoma) and malignant Schwannoma, as well as tumours of the central nervous system such as brain and bone marrow tumours; intestinal cancer such as for example carcinoma of the rectum, colon, anus, small intestine and duodenum; eyelid tumours such as basalioma or basal cell carcinoma; pancreatic cancer or carcinoma of the pancreas; bladder cancer or carcinoma of the bladder; lung cancer (bronchial carcinoma) such as for example small-cell bronchial carcinomas (oat cell carcinomas) and non-small cell bronchial carcinomas such as plate epithelial carcinomas, adenocarcinomas and large-cell bronchial carcinomas; breast cancer such as for example mammary carcinoma such as infiltrating ductal carcinoma, colloid carcinoma, lobular invasive carcinoma, tubular carcinoma, adenocystic carcinoma and papillary carcinoma; non-Hodgkin's lymphomas (NHL) such as for example Burkitt's lymphoma, low-malignancy non-Hodgkin's lymphomas (NHL) and mucosis fungoides; uterine cancer or endometrial carcinoma or corpus carcinoma; CUP syndrome (Cancer of Unknown Primary); ovarian cancer or ovarian carcinoma such as mucinous, endometrial or serous cancer; gall bladder cancer; bile duct cancer such as for example Klatskin tumour; testicular cancer such as for example seminomas and non-seminomas; lymphoma (lymphosarcoma) such as for example malignant lymphoma, Hodgkin's disease, non-Hodgkin's lymphomas (NHL) such as chronic lymphatic leukaemia, leukaemic reticuloendotheliosis, immunocytoma, plasmocytoma (multiple myeloma), immunoblastoma, Burkitt's lymphoma, T-zone mycosis fungoides, large-cell anaplastic lymphoblastoma and lymphoblastoma; laryngeal cancer such as for example tumours of the vocal cords, supraglottal, glottal and subglottal laryngeal tumours; bone cancer such as for example osteochondroma, chondroma, chondroblastoma, chondromyxoid fibroma, osteoma, osteoid osteoma, osteoblastoma, eosinophilic granuloma, giant cell tumour, chondrosarcoma, osteosarcoma, Ewing's sarcoma, reticulo-sarcoma, plasmocytoma, giant cell tumour, fibrous dysplasia, juvenile bone cysts and aneurysmatic bone cysts; head and neck tumours such as for example tumours of the lips, tongue, floor of the mouth, oral cavity, gums, palate, salivary glands, throat, nasal cavity, paranasal sinuses, larynx and middle ear; liver cancer such as for example liver cell carcinoma or hepatocellular carcinoma (HCC); leukaemias, such as for example acute leukaemias such as acute lymphatic/lymphoblastic leukaemia (ALL), acute myeloid leukaemia (AML); chronic leukaemias such as chronic lymphatic leukaemia (CLL), chronic myeloid leukaemia (CML); stomach cancer or gastric carcinoma such as for example papillary, tubular and mucinous adenocarcinoma, signet ring cell carcinoma, adenosquamous carcinoma, small-cell carcinoma and undifferentiated carcinoma; melanomas such as for example superficially spreading, nodular, lentigo-maligna and acral-lentiginous melanoma; renal cancer such as for example kidney cell carcinoma or hypernephroma or Grawitz's tumour; oesophageal cancer or carcinoma of the oesophagus; penile cancer; prostate cancer; throat cancer or carcinomas of the pharynx such as for example nasopharynx carcinomas, oropharynx carcinomas and hypopharynx carcinomas; retinoblastoma; vaginal cancer or vaginal carcinoma; plate epithelial carcinomas, adenocarcinomas, in situ carcinomas, malignant melanomas and sarcomas; thyroid carcinomas such as for example papillary, follicular and medullary thyroid carcinoma, as well as anaplastic carcinomas; spinalioma, epidormoid carcinoma and plate epithelial carcinoma of the skin; thymomas, cancer of the urethra and cancer of the vulva.

The new compounds may be used for the prevention, short-term or long-term treatment of the above-mentioned diseases, also optionally in combination with other “state-of-the-art” compounds, such as other anti-tumour substances, cytotoxic substances, cell proliferation inhibitors, anti-angiogenic substances, steroids or antibodies.

The compounds of general formula (1) may be used on their own or in combination with other active substances according to the invention, optionally also in combination with other pharmacologically active active substances.

Chemotherapeutic agents which may be administered in combination with the compounds according to the invention, include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortinsone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors (growth factors such as for example “platelet derived growth factor” and “hepatocyte growth factor”, inhibitors are for example “growth factor” antibodies, “growth factor receptor” antibodies and tyrosinekinase inhibitors, such as for example gefitinib, imatinib, lapatinib and trastuzumab); antimetabolites (e.g. antifolates such as methotrexate, raltitrexed, pyrimidine analogues such as 5-fluorouracil, capecitabin and gemcitabin, purine and adenosine analogues such as mercaptopurine, thioguanine, cladribine and pentostatin, cytarabine, fludarabine); antitumour antibiotics (e.g. anthracyclins such as doxorubicin, daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); topoisomerase inhibitors (e.g. epipodophyllotoxins such as for example etoposide and etopophos, teniposide, amsacrin, topotecan, irinotecan, mitoxantron) and various chemotherapeutic agents such as amifostin, anagrelid, clodronat, filgrastin, interferon alpha, leucovorin, rituximab, procarbazine, levamisole, mesna, mitotane, pamidronate and porfimer.

Suitable preparations include for example tablets, capsules, suppositories, solutions,—particularly solutions for injection (s.c., i.v., i.m.) and infusion—elixirs, emulsions or dispersible powders. The content of the pharmaceutically active compound(s) should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by oral or transdermal route, most preferably by oral route. For oral administration the tablets may, of course contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

The formulation examples which follow illustrate the present invention without restricting its scope:

Examples of Pharmaceutical Formulations

| A) | Tablets | per tablet |
|---|---|---|
| | active substance | 100 mg |
| | lactose | 140 mg |
| | corn starch | 240 mg |
| | polyvinylpyrrolidone | 15 mg |
| | magnesium stearate | 5 mg |
| | | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) | Tablets | per tablet |
|---|---|---|
| | active substance | 80 mg |
| | lactose | 55 mg |
| | corn starch | 190 mg |
| | microcrystalline cellulose | 35 mg |
| | polyvinylpyrrolidone | 15 mg |
| | sodium-carboxymethyl starch | 23 mg |
| | magnesium stearate | 2 mg |
| | | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

| C) | Ampoule solution | | |
|---|---|---|---|
| | active substance | 50 | mg |
| | sodium chloride | 50 | mg |
| | water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

1.) A compound of formula (1), 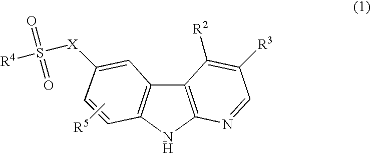
wherein
X is equal to O, NR¹ or CHR¹, and
R¹ denotes a group selected from among hydrogen, C₁₋₃alkyl and C₁₋₃haloalkyl, and
R² and R³ each independently of one another denote hydrogen or a group selected from among R^{a}, R^{b} and R^{a} substituted by one or more identical or different R^{b} and/or R^{c} and
R⁴ denotes —NR^{c}R^{c} or a group, optionally substituted by one or more R⁶, selected from among C₁₋₆alkyl, C₃₋₁₀cycloalkyl, 3-8 membered heterocyclyl, C₆₋₁₄aryl and 5-15 membered heteroaryl, and
R⁵ denotes a group selected from among hydrogen, halogen, C₁₋₃alkyl and C₁₋₃haloalkyl, and
R⁶ denotes a group selected from among R^{a}, R^{b} and R^{a} substituted by one or more identical or different R^{b} and/or R^{c}, and
each R^{a} independently of one another selected from among C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocyclyl, 4-14 membered heterocyclylalkyl, 5-10 membered heteroaryl and 6-16 membered heteroarylalkyl, and
each R^{b} denotes a suitable group and each independently of one another selected from among ═O, —OR^{d}, C₁₋₃haloalkyloxy, —OCF₃, ═S, —SR^{d}, ═NR^{d}, ═NOR^{d}, —NR^{c}R^{c}, halogen, —CF3, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(S)R^{d}, —C(O)OR^{d}, —C(O)NR^{c}R^{c}, —C(O)NR^{d}OR^{d}, —C(O)N(R^{d})NR^{c}R^{c}, —CN(R^{d})NR^{c}R^{c}, —CN(OH)R^{d}, —CN(OH)NR^{c}R^{c}, —OC(O)R^{d}, —OC(O)OR^{d}, —OC(O)NR^{c}R^{c}, —OCN(R^{d})NR^{c}R^{c}, —N(R^{d})C(O)R^{d}, —N(R^{d})C(S)R^{d}, —N(R^{d})S(O)₂R^{d}, —N(R^{d})C(O)OR^{d}, —N(R^{d})C(O)NR^{c}R^{c}, and —N(R^{d})C(NR^{d})NR^{c}R^{c}, and
each R^{e} independently of one another denotes hydrogen or a group optionally substituted by one or more identical or different R^{d} and/or R^{e} selected from among C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocyclyl, 4-14 membered heterocyclylalkyl, 5-10 membered heteroaryl and 6-16 membered heteroarylalkyl; and
each R^{d} independently of one another denotes hydrogen or a group optionally substituted by one or more identical or different R^{e} and/or R^{f} selected from among C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocyclyl, 4-14 membered heterocyclylalkyl, 5-10 membered heteroaryl and 6-16 membered heteroarylalkyl;
each R^{e} denotes a suitable group and each independently of one another selected from among ═O, —OR^{g}, C₁₋₃haloalkyloxy, —OCF₃, ═S, —SR^{g}, ═NR^{g}, ═NOR^{g}, —NR^{f}R^{f}, halogen, —CF3, —CN, —NC, —OCN, —SCN, —NO, —NO₂, ═N₂, —N₃, —S(O)R^{g}, —S(O)₂R^{g}, —S(O)₂OR^{g}, —S(O)NR^{f}R^{f}, —S(O)₂NR^{f}R^{f}, —OS(O)R^{g}, —OS(O)₂R^{g}, —OS(O)₂OR^{g}, —OS(O)₂NR^{f}R^{f}, —C(O)R^{g}, —C(O)OR^{g}, —C(O)NR^{f}R^{f}, —CN(R^{g})NR^{f}R^{f}, —CN(OH)R^{g}, —C(NOH)NR^{f}R^{f}, —OC(O)R^{g}, —OC(O)OR^{g}, —OC(O)NR^{f}R^{f}, —OCN(R^{g})NR^{f}R^{f}, —N(R^{g})C(O)R^{g}, —N(R^{g})C(S)R^{g}, —N(R^{g})S(O)₂R^{g}, —N(R^{g})C(O)OR^{g}, —N(R^{g})C(O)NR^{f}R^{f}, and —N(R^{g})C(NR^{g})NR^{f}R^{f}, and
each R^{f} independently of one another denotes hydrogen or a group optionally substituted by one or more identical or different R^{g} selected from among C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocyclyl, 4-14 membered heterocyclylalkyl, 5-10 membered heteroaryl and 6-16 membered heteroarylalkyl, and
each R^{g} independently of one another denotes hydrogen, C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocyclyl, 4-14 membered heterocyclylalkyl, 5-10 membered heteroaryl and 6-16 membered heteroarylalkyl,
or a tautomer, or pharmacologically acceptable salt thereof.

2.) A compound according to claim 1, wherein R² denotes a group selected from among C₃₋₁₀cycloalkyl, 3-8 membered heterocyclyl, C₆₋₁₄aryl and 5-10 membered heteroaryl.

3.) A compound according to claim 2, wherein R² denotes a group selected from among phenyl and pyridyl.

4.) A compound according to claim 1, wherein R³ denotes phenyl.

5.) A compound according to claim 1, wherein R⁴ denotes a group selected from among C₁₋₆alkyl, C₆₋₁₄aryl, 3-8 membered heterocyclyl and 5-10 membered heteroaryl.

6.) A compound according to claim 1, wherein R⁴ denotes a group selected from among phenyl, isoxazolyl, thienyl and imidazolyl.

7.) A pharmaceutical composition comprising one or more compounds of formula (1) according to claim 1 or a pharmacologically acceptable salt thereof, optionally in combination with an excipient and/or carrier.

8.) A method for treating and/or preventing cancer, infection, or an inflammatory or autoimmune disease in a subject comprising administering to said subject a therapeutically effective amount of a compound according to claim 1.

9.) A pharmaceutical composition comprising a compound according to claim 1 and at least one other cytostatic or cytotoxic active substance different from formula (1).
